SEQUENCE LISTING (14 sequences)

SEQ ID NO: 1 - RNA, 27 nt, Artificial Sequence
  Sense strand of gp46siRNAseqA
guuccaccauaagaugguagacaacag

SEQ ID NO: 2 - RNA, 27 nt, Artificial Sequence
  Antisense strand of gp46siRNAseqA
guugucuaccaucuuaugguggaacau

SEQ ID NO: 3 - RNA, 27 nt, Artificial Sequence
  Sense strand of gp46siRNAseqB
ccacaaguuuuauauccaaucuagcag

SEQ ID NO: 4 - RNA, 27 nt, Artificial Sequence
  Antisense strand of gp46siRNAseqB
gcuagauuggauauaaaacuuguggau

SEQ ID NO: 5 - RNA, 27 nt, Artificial Sequence
  Sense strand of gp46siRNAseqC
cuagagccauuacauuacauugacaag

SEQ ID NO: 6 - RNA, 27 nt, Artificial Sequence
  Antisense strand of gp46siRNAseqC
ugucaauguaauguaauggcucuagau

SEQ ID NO: 7 - RNA, 27 nt, Artificial Sequence
  Sense strand of random siRNA
cgauucgcuagaccggcuucauugcag

SEQ ID NO: 8 - RNA, 27 nt, Artificial Sequence
  Antisense strand of random siRNA
gcaaugaagccggucuagcgaaucgau

SEQ ID NO: 9 - DNA, 21 bp, Artificial Sequence
  Synthetic oligonucleotide
ggacaggccucuacaacuatt

SEQ ID NO: 10 - DNA, 21 bp, Artificial Sequence
  Synthetic oligonucleotide
ttccuguccggagauguugau

SEQ ID NO: 11 - DNA, 21 bp, Artificial Sequence
  Synthetic oligonucleotide
ggacaggccuguacaacuatt

SEQ ID NO: 12 - DNA, 21 bp, Artificial Sequence
  Synthetic oligonucleotide
ttccuguccggacauguugau

SEQ ID NO: 13 - RNA, 19 nt, Artificial Sequence
  Artificially synthesized RNA molecule
nagacacaugggugcuaun

SEQ ID NO: 14 - RNA, 20 nt, Artificial Sequence
  Artificially synthesized RNA molecule
uauagcacccaugugucucn

(19) United States
(12) Patent Application Publication
(10) Pub. No.: US 2013/0210744 A1
(43) Pub. Date: Aug. 15, 2013

(54) TARGETING AGENT FOR CANCER CELL OR CANCER-ASSOCIATED FIBROBLAST

(71) Applicant: Nitto Denko Corporation, (US)

(72) Inventors: Yoshiro Niitsu, Sapporo-shi (JP); Rishu Takimoto, Sapporo-shi (JP); Joseph E. Payne, Oceanside, CA (US); John A. Gaudette, Poway, CA (US); Zheng Hou, San Diego, CA (US); Victor Knopov, Oceanside, CA (US); Richard P. Witte, San Diego, CA (US); Mohammad Ahmadian, Carlsbad, CA (US); Loren A. Perelman, San Diego, CA (US); Yasunobu Tanaka, Osaka (JP); Violetta Akopian, Oceanside, CA (US)

(73) Assignee: NITTO DENKO CORPORATION, Osaka (JP)

(21) Appl. No.: 13/836,060

(22) Filed: Mar. 15, 2013

Related U.S. Application Data
(63) Continuation-in-part of application No. 12/450,571, filed on Feb. 22, 2010.
Application No. 12/450,571 is a continuation-in-part of application No. 13/492,424, filed on Jun. 8, 2012.
(60) Provisional application No. 61/494,840, filed on Jun. 8, 2011.

(30) Foreign Application Priority Data
Mar. 30, 2007 (JP) 2007-091808
Oct. 4, 2007 (JP) 2007-261202
Dec. 17, 2007 (JP) 2007-324459

(51) Int. Cl. A61K 47/48

(52) U.S. Cl. CPC A61K 47/48338; A61K 47/48053; A61K 47/48215; A61K 47/48046
USPC 514/19.3; 554/80; 514/119; 530/330; 554/106; 514/616; 554/110; 514/548

(57) ABSTRACT

Disclosed are a novel therapeutic agent and a novel treatment method for cancer. Specifically disclosed are: a targeting agent for a cell selected from the group consisting of a cancer cell and a cancer-associated fibroblast, which comprises a retinoid and/or derivative thereof; a substance delivery carrier for the cell, which comprises the targeting agent; an anti-cancer composition utilizing the targeting agent or the carrier; an anticancer-associated fibroblast composition; and a method for treatment of cancer.

### CROSS-REFERENCE TO RELATED APPLICATIONS

This application is a continuation-in-part of U.S. Ser. No. 12/450,571, filed Feb. 22, 2010, which is a national stage filing under 35 U.S.C. §371 of international application PCT/JP2008/056735, filed Mar. 28, 2008. This application is also a continuation-in-part of U.S. Ser. No. 13/492,424, filed Jun. 8, 2012, which claims the benefit of U.S. Provisional Application No. 61/494,840 filed Jun. 8, 2011. The disclosures of all of the above are hereby incorporated by reference in their entireties for all purposes.

### SEQUENCE LISTING

The present application is being filed along with a Sequence Listing in electronic format. The Sequence Listing is provided as a file entitled KUZU1_{—}001P1.TXT, created Mar. 15, 2013, which is 7 KB in size. The information in the electronic format of the Sequence Listing is incorporated herein by reference in its entirety.

### BACKGROUND OF THE INVENTION

1. Field of the Invention

The present invention relates to a targeting agent to a cell selected from the group consisting of a cancer cell and a cancer-associated fibroblast (CAF: cancer-associated fibroblast or carcinoma-associated fibroblast), a substance delivery carrier to the cell, the carrier containing the targeting agent, and an anticancer composition, an anti-CAF composition, and a method for treating a cancer utilizing same. The present invention is further directed to the use of fat-soluble vitamin compounds to target and enhance activity of therapeutic molecules, including siRNA.

2. Description of the Related Art

Cancer is one of the most significant diseases confronting mankind, and much research effort is going into the treatment thereof. In cancer treatment, particularly in the medical therapy of cancer, various anticancer agents for suppressing the growth of cancer cells have been developed, and some degree of success has been achieved, but since such drugs suppress the growth of not only cancer cells but also normal cells, there are problems with various side effects such as nausea and vomiting, hair loss, myelosuppression, kidney damage, and nerve damage. As an approach to reduce such side effects, attempts have been made in recent years to specifically deliver an anticancer agent to cancer cells or cancer tissue. By specific delivery of an anticancer agent, it is not only possible to prevent the anticancer agent from reaching normal cells and reduce the side effects, but also to obtain the economic benefit that the dose of the anticancer agent can be decreased.

As a concrete example of a delivery method, there have been developed techniques such as passive targeting in which the EPR (enhanced permeability and retention) effect is utilized and active targeting in which a drug is modified by a ligand for a surface molecule that is specifically expressed on cancer cells. As molecules that can be utilized in active targeting, molecules that are endocytosed into cells as a result of ligand bonding, such as, for example, CD19, HER2, a transferrin receptor, a folate receptor, a VIP receptor, EGFR (Nonpatent Publication 1), RAAG10 (Patent Publication 1), PIPA (Patent Publication 2), and KID3 (Patent Publication 3) have been reported. However, none of the delivery methods are yet satisfactory, and there has been a further desire for the development of cancer cell-specific delivery methods.

Furthermore, in the medical therapy of cancer, from the idea that a cancer can be cured by killing the cancer cells themselves, various anticancer agents targeted at cancer cells have been developed and used. However, such attempts could not always achieve satisfactory results because of the above-mentioned problems with side effects, or the occurrence of additional phenomena such as relapse due to minimal residual disease, resistance of tumor cells to the anticancer agent, etc.

On the other hand, as a result of recent research, it has gradually become clear that the environment around a cancer, for example, interstitial tissue which includes blood vessels, ECM, and fibroblasts, plays an important role in the onset and progression of the cancer. For example, Camps et al. (see Nonpatent Publication 2) reported that when an athymic nude mouse was inoculated with tumor cells that do not form a tumor on their own or for which the tumor formation rate is low, together with tumorigenic fibroblasts, rapid and marked formation of a tumor was observed, and Olumi et al. (see Nonpatent Publication 3) reported that when peritumoral fibroblasts (i.e. CAFs) from a prostate tumor patient were grafted on an athymic animal together with human prostate cells, the neoplastic growth thereof was markedly accelerated. Furthermore, it has been clarified that a bioactive substance such as PDGF (platelet-derived growth factor), TGF-β (transforming growth factor-β), HGF (hepatocyte growth factor), or SDF-1 (stromal cell-derived factor-1) produced in the interstitium is involved in such growth of a tumor (see Nonpatent Publication 4).

From these findings, the importance of the environment around a cancer has been brought to the fore, and new treatment methods that, rather than the cancer cells themselves, are targeted at the environment around them have been investigated. Among them, CAFs, which secrete various bioactive substances and are deeply involved in the onset and progression of cancer, have been attracting attention in recent years, but fundamental research thereinto only has a short history of 10 or so years, and although some of the cancer treatment methods that are targeted at bioactive substances secreted from CAFs have been recognized as having some degree of effect, in the current situation none is recognized as having any effect as a cancer treatment method targeted at CAFs themselves (see Nonpatent Publication 4).

### CITATION LIST

Patent Publication 1. JP 2005-532050 A
Patent Publication 2. JP 2006-506071 A
Patent Publication 3. JP 2007-529197 A
Patent Publication 4. WO 2006/068232
Nonpatent Publication 1. Torchilin, AAPS J. 2007; 9(2): E128-47
Nonpatent Publication 2. Camps et al., Proc Natl Acad Sci USA. 1990; 87(1): 75-9
Nonpatent Publication 3. Olumi et al., Cancer Res. 1999; 59(19): 5002-11
Nonpatent Publication 4. Micke et al., Expert Opin Ther Targets. 2005; 9(6): 1217-33

### SUMMARY OF THE INVENTION

### Problems to be Solved by the Invention

It is an object of the present invention to provide a carrier that can deliver a substance such as a drug specifically to a cancer cell, and a cancer drug and a cancer treatment method utilizing same, and also to provide a carrier that can deliver a drug specifically to a CAF, and a cancer drug and a cancer treatment method utilizing same.

### Means for Solving the Problems

While searching for a novel cancer treatment method, the present inventors have found that there is not yet a carrier that can deliver a drug specifically to CAFs, and as a result of continuing an intensive investigation in order to develop such a carrier, it has been found that a carrier containing a retinoid as a targeting agent specifically accelerates drug delivery to CAFs. As a result of further investigation into the above carrier, it has been found that the carrier also specifically accelerates the delivery of a substance to cancer cells, and the present invention has thus been accomplished.

It is known that a carrier containing retinol delivers a drug to stellate cells storing retinol (see Patent Publication 4), but it was not known until now that it specifically accelerates the delivery of a drug to cancer cells or CAFs.

That is, in one aspect, the present invention relates to:

(i) a targeting agent to a cell selected from the group consisting of a cancer cell and a cancer-associated fibroblast, the targeting agent including a retinoid;

(ii) the targeting agent of (i), wherein the retinoid includes retinol;

(iii) a substance delivery carrier to a cell selected from the group consisting of a cancer cell and a cancer-associated fibroblast, the carrier including the targeting agent of (i) or (ii);

(iv) the carrier of (iii), wherein the content of the targeting agent is 0.2 to 20 wt % of the entire carrier;

(v) the carrier of (iii) or (iv), wherein the molar ratio of the targeting agent to constituent components of the carrier other than the targeting agent is 8:1 to 1:4;

(vi) an anticancer composition that includes the targeting agent of (i) or (ii) or the carrier of any one of (iii) to (v), and a drug that controls the activity or growth of a cancer cell and/or a cancer-associated fibroblast;

(vii) an anti-cancer-associated fibroblast composition that includes the targeting agent of (i) or (ii) or the carrier of any one of (iii) to (v), and a drug that controls the activity or growth of a cancer-associated fibroblast;

(viii) the composition of (vi), wherein the drug that controls the activity or growth of a cancer cell is an anticancer agent;

(ix) the composition of any one of (vi) to (viii), wherein the drug that controls the activity or growth of a cancer-associated fibroblast is selected from the group consisting of an inhibitor of activity or production of a bioactive substance selected from the group consisting of TGF-β, HGF, PDGF, VEGF (vascular endothelial growth factor), IGF (insulin-like growth factor), MMP (matrix metalloproteinase), FGF (fibroblast growth factor), uPA (urokinase-type plasminogen activator), cathepsin, and SDF-1, a cell activity suppressor, a growth inhibitor, an apoptosis inducer, and an siRNA, ribozyme, antisense nucleic acid, DNA/RNA chimeric polynucleotide, or vector expressing same that targets one or more molecules from among an extracellular matrix constituent molecule produced by cancer-associated fibroblasts and a molecule involved in the production or secretion of the extracellular matrix constituent molecule;

(x) the composition of (ix), wherein the molecule involved in the production or secretion of the extracellular matrix constituent molecule is HSP47;

(xi) the composition of any one of (vi) to (x), wherein the drug and the targeting agent or the carrier are mixed at a place of medical treatment or in the vicinity thereof; and

(xii) a preparation kit for the composition of any one of (vi) to (xi), the kit including one or more containers containing singly or in combination the drug, the targeting agent, and as necessary carrier constituent substances other than the targeting agent.

In one embodiment, the retinoid is provided as a compound containing one or more retinoid moieties, such as a compound consisting of the structure (retinoid)m-linker-(retinoid)n, wherein m and n are independently 0, 1, 2, or 3, except that m and n are not both zero; and wherein the linker comprises a polyethylene glycol (PEG) or PEG-like molecule, or a compound consisting of the structure (lipid)m-linker-(retinoid)n, wherein m and n are independently 0, 1, 2, or 3, except that m and n are not both zero; and wherein the linker comprises a polyethylene glycol (PEG) molecule.

In another aspect, the present invention provides a compound for facilitating drug delivery to a target cell, consisting of the structure (targeting molecule)ₘ-linker-(targeting molecule)ₙ, wherein the targeting molecule is a retinoid having a specific receptor or activation/binding site on the target cell; wherein m and n are independently 0, 1, 2 or 3; and wherein the linker comprises a polyethylene glycol (PEG) or PEG-like molecule. In an embodiment, m and n are not both zero.

In one embodiment, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

In another embodiment, the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

In another embodiment, the compound is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

In another embodiment, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

In another embodiment, the compound is a composition of the formula

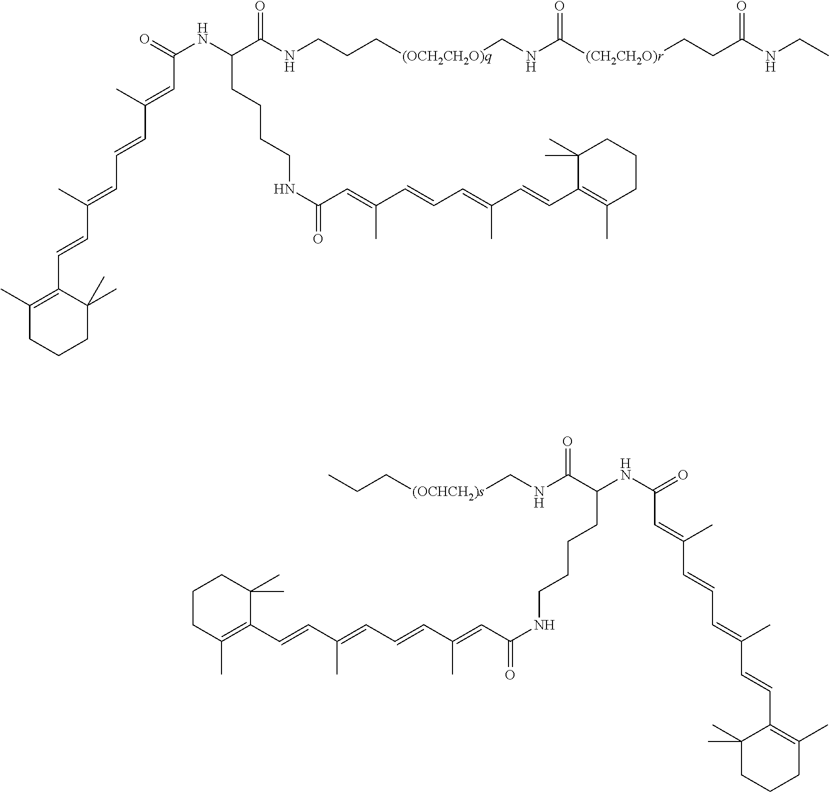

wherein q, r, and s are each independently 1, 2, 3, 4, 5, 6, 7, 8, 9, or 10.

In another embodiment in which q, r and s are 3, 5 and 3, respectively, the formula of the compound comprises

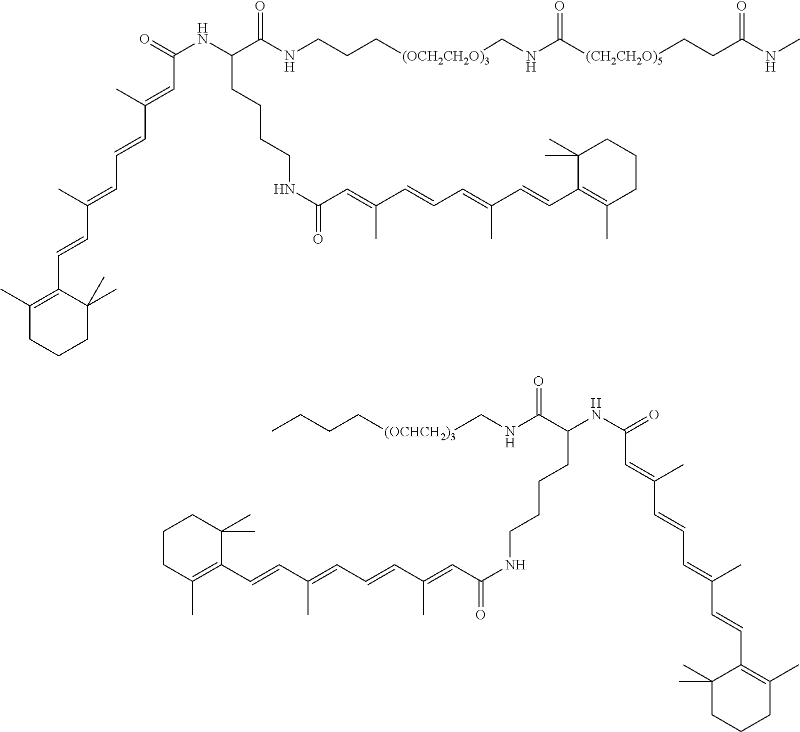

In another aspect, the present invention provides a stellate-cell-specific drug carrier comprising a stellate cell specific amount of a retinoid molecule consisting of the structure (retinoid)ₘ-linker-(retinoid)ₙ; wherein m and n are independently 0, 1, 2 or 3; and wherein the linker comprises a polyethylene glycol (PEG) or PEG-like molecule. In an embodiment, m and n are not both zero.

In another embodiment, the present invention provides a composition comprising a liposomal composition. In other embodiments, the liposomal composition comprises a lipid vesicle comprising a bilayer of lipid molecules.

In certain embodiments, the retinoid molecule is at least partially exposed on the exterior of the drug carrier before the drug carrier reaches the stellate cell.

In another embodiment, the retinoid is 0.1 mol % to 20 mol % of the lipid molecules. The retinoid will be present in a concentration of about 0.3 to 30 weight percent, based on the total weight of the composition or formulation, which is equivalent to about 0.1 to about 10 mol %.

The present invention also provides embodiments where the lipid molecules comprise one or more lipids selected from the group consisting of HEDC, DODC, HEDODC, DSPE, DOPE, and DC-6-14. In another embodiment, the lipid molecules further comprise S104.

In certain embodiments, the drug carrier comprises a nucleic acid.

In other embodiments, the nucleic acid is an siRNA that is capable of knocking down expression of hsp47 mRNA in the stellate cell.

In another aspect, the present invention provides a compound for facilitating drug delivery to a target cell, consisting of the structure (lipid)ₘ-linker-(targeting molecule)ₙ, wherein the targeting molecule is a retinoid or a fat soluble vitamin having a specific receptor or activation/binding site on the target cell; wherein m and n are independently 0, 1, 2 or 3; and wherein the linker comprises a polyethylene glycol (PEG) molecule. In an embodiment, m and n are not both zero.

In one embodiment, the lipid is selected from one or more of the group consisting of DODC, HEDODC, DSPE, DOPE, and DC-6-14.

In another embodiment, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

In another embodiment of the present invention, the fat-soluble vitamin is vitamin D, vitamin E, or vitamin K.

In another embodiment, the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

In another embodiment the present invention is selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

Accordingly, the present invention provides the following:

(1) A targeting agent to a cancer cell, the targeting agent comprising one or more compounds selected from the group consisting of a retinoid, (retinoid)ₘ-linker-(retinoid)ₙ and (lipid)ₘ-linker-(retinoid)ₙ, wherein m and n are independently 0, 1, 2, or 3, except that m and n are not both zero; and wherein the linker comprises a polyethylene glycol (PEG) or PEG-like molecule.

(2) The targeting agent according to (1), wherein at least one of the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(3) The targeting agent according to (1) or (2), wherein the retinoid is retinol.

(4) The targeting agent according to any one of (1) to (3), wherein the linker of the (retinoid)ₘ-linker-(retinoid)ₙ is selected from the group consisting of bis-amido-PEG, tris-amido-PEG, tetra-amido-PEG, Lys-bis-amido-PEG-Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

(5) The targeting agent according to any one of (1) to (4), wherein the (retinoid)ₘ-linker-(retinoid)ₙ is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

(6) The targeting agent according to any one of (1) to (5), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(7) The targeting agent according to any one of (1) to (6), wherein the targeting agent comprises a compound of formula

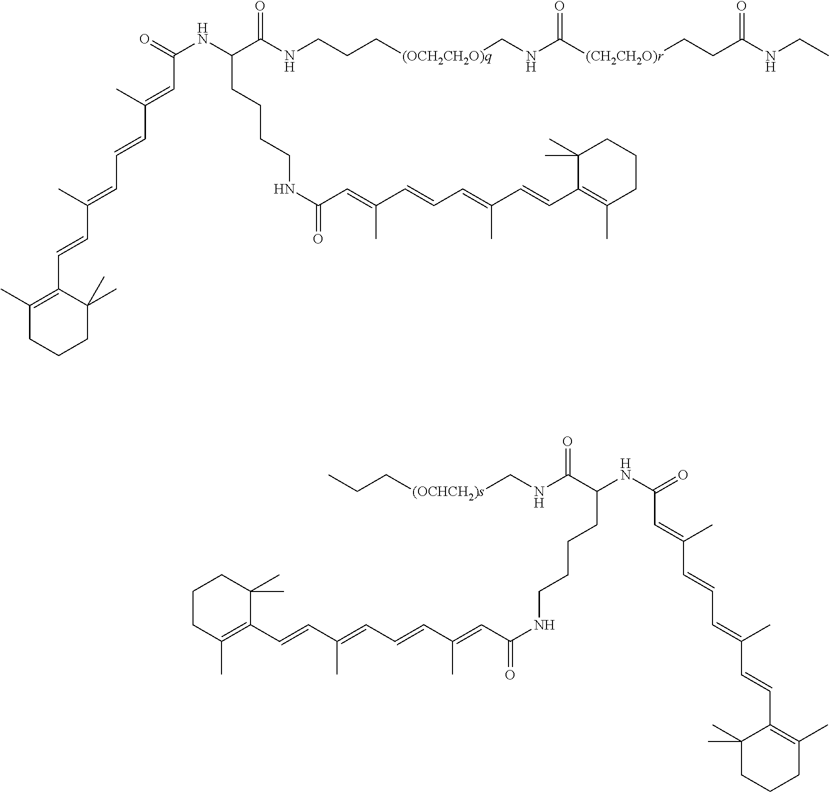

wherein q, r, and s are each independently 1, 2, 3, 4, 5, 6, 7, 8, 9, or 10.

(8) The targeting agent according to any one of (1) to (7), wherein the targeting agent comprises a compound in which q, r and s are 3, 5 and 3, respectively, of formula

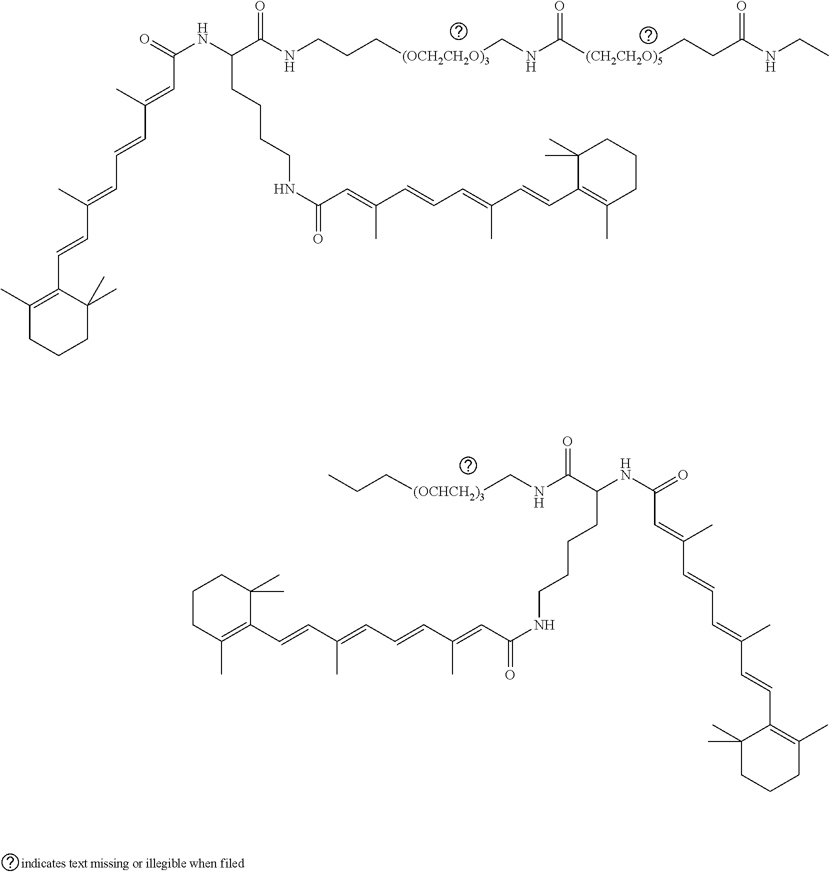

(9) The targeting agent according to (1), wherein the lipid is selected from one or more of the group consisting of DODC, HEDODC, DSPE, DOPE, and DC-6-14.

(10) The targeting agent according to any one of (1) or (9), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(11) The targeting agent according to any one of (1) and (9) to (10), wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

(12) The targeting agent according to any one of (1) and (9) to (11), selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

(13) The targeting agent according to any one of (1) and (9) to (12), wherein the lipid moieties comprise one or more lipids selected from the group consisting of HEDC, DODC, HEDC, HEDODC, DSPE, DOPE, and DC-6-14.

(14) The targeting agent according to any one of (1) and (9) to (13), wherein the lipid moieties further comprise S104.

(15) A substance delivery carrier to a cancer cell, the carrier comprising the targeting agent according to any one of (1) to (14).

(16) The carrier according to (15), wherein the content of the targeting agent is 0.2 to 20 wt % of the entire carrier.

(17) The carrier according to (15) or (16), wherein the molar ratio of the targeting agent to constituent components of the carrier other than the targeting agent is 8:1 to 1:4.

(18) An anticancer composition comprising the targeting agent according to any one of (1) to (14), and a drug that controls the activity or growth of a cancer cell.

(19) The composition according to (18), wherein the drug that controls the activity or growth of a cancer cell is an anticancer agent.

(20) An anticancer composition comprising the carrier according to any one of (15) to (17), and a drug that controls the activity or growth of a cancer cell.

(21) The composition according to (20), wherein the drug that controls the activity or growth of a cancer cell is an anticancer agent.

(22) The composition according to (18) or (19), wherein the drug and the targeting agent are mixed at a place of medical treatment or in the vicinity thereof.

(23) The composition according to (20) or (21), wherein the drug and the carrier are mixed at a place of medical treatment or in the vicinity thereof.

(24) A preparation kit for the composition according to any one of (18) to (23), wherein it comprises one or more containers comprising singly or in combination the drug, the targeting agent, and as necessary carrier constituent substances other than the targeting agent.

(25) A targeting agent to a cancer-associated fibroblast, the targeting agent comprising one or more compounds selected from the group consisting of a retinoid, (retinoid)ₘ-linker-(retinoid)ₙ and (lipid)ₘ-linker-(retinoid)ₙ, wherein m and n are independently 0, 1, 2, or 3, except that m and n are not both zero; and wherein the linker comprises a polyethylene glycol (PEG) or PEG-like molecule.

(26) The targeting agent according to (25), wherein at least one of the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(27) The targeting agent according to (25) or (26), wherein the retinoid is retinol.

(28) The targeting agent according to any one of (25) to (27), wherein the linker of the (retinoid)ₘ-linker-(retinoid)ₙ is selected from the group consisting of bis-amido-PEG, tris-amido-PEG, tetra-amido-PEG, Lys-bis-amido-PEG-Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

(29) The targeting agent according to any one of (25) to (28), wherein the (retinoid)ₘ-linker-(retinoid)ₙ is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

(30) The targeting agent according to any one of (25) to (29), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(31) The targeting agent according to any one of (25) to (30), wherein the targeting agent comprises a compound of formula

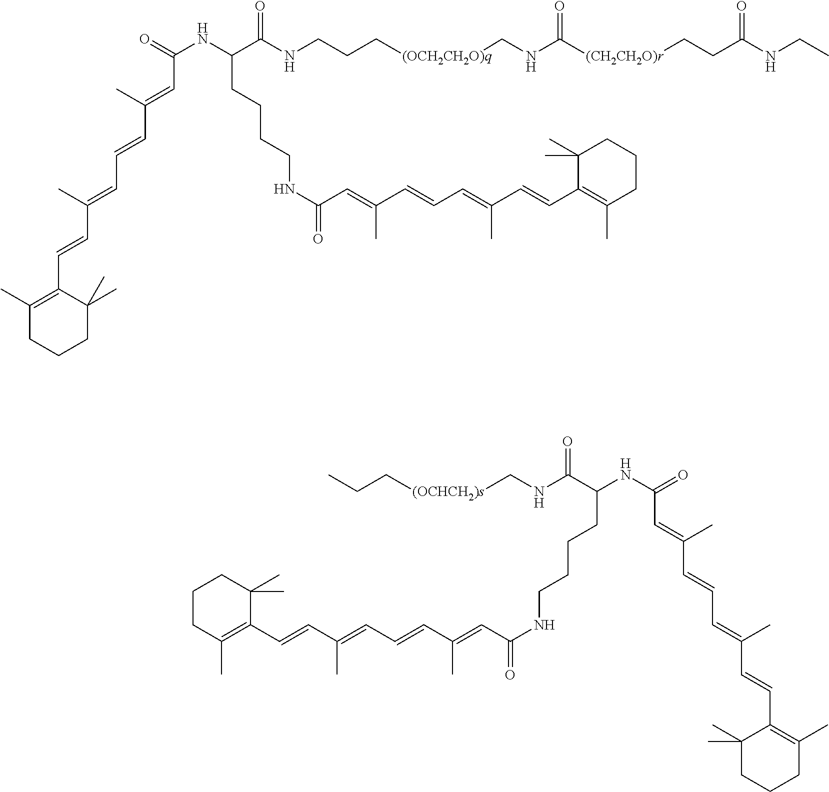

wherein q, r, and s are each independently 1, 2, 3, 4, 5, 6, 7, 8, 9, or 10.

(32) The targeting agent according to any one of (25) to (31), wherein the targeting agent comprises a compound in which q, r and s are 3, 5 and 3, respectively, of formula

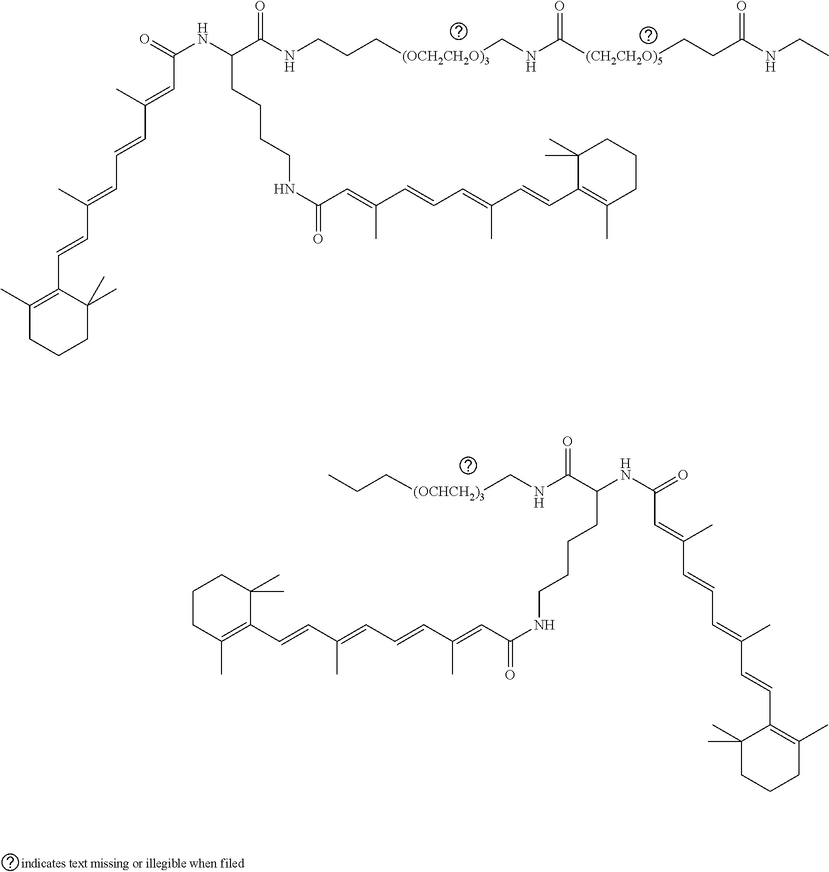

(33) The targeting agent according to (25), wherein the lipid is selected from one or more of the group consisting of DODC, HEDODC, DSPE, DOPE, and DC-6-14.

(34) The targeting agent according to any one of (25) or (33), wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

(35) The targeting agent according to any one of (25), (33) and (34), wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6 (hexyl), Gly3, and GluNH.

(36) The targeting agent according to any one of (25) and (33) to (35), selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

(37) The targeting agent according to any one of (25) and (33) to (36), wherein the lipid moieties comprise one or more lipids selected from the group consisting of HEDC, DODC, HEDC, HEDODC, DSPE, DOPE, and DC-6-14.

(38) The targeting agent according to any one of (25) and (33) to (37), wherein the lipid moieties further comprise S 104.

(39) A substance delivery carrier to a cancer-associated fibroblast, the carrier comprising the targeting agent according to any one of (25) to (38).

(40) The carrier according to (39), wherein the content of the targeting agent is 0.2 to 20 wt % of the entire carrier.

(41) The carrier according to (39) or (40), wherein the molar ratio of the targeting agent to constituent components of the carrier other than the targeting agent is 8:1 to 1:4.

(42) An anti-cancer-associated fibroblast composition comprising the targeting agent according to any one of (25) to (38), and a drug that controls the activity or growth of a cancer-associated fibroblast.

(43) The composition according to (42), wherein the drug that controls the activity or growth of a cancer-associated fibroblast is selected from the group consisting of an inhibitor of activity or production of a bioactive substance selected from the group consisting of TGF-α, HGF, PDGF, VEGF, IGF, MMP, FGF, uPA, cathepsin, and SDF-1, a cell activity suppressor, a growth inhibitor, an apoptosis inducer, and an siRNA, ribozyme, antisense nucleic acid, DNA/RNA chimeric polynucleotide, or vector expressing same that targets one or more molecules from among an extracellular matrix constituent molecule produced by cancer-associated fibroblasts and a molecule involved in the production or secretion of the extracellular matrix constituent molecule.

(44) The composition according to (43), wherein the molecule involved in the production or secretion of the extracellular matrix constituent molecule is HSP47.

(45) An anti-cancer-associated fibroblast composition comprising the carrier according to any one of (39) to (41), and a drug that controls the activity or growth of a cancer-associated fibroblast.

(46) The composition according to (45), wherein the drug that controls the activity or growth of a cancer-associated fibroblast is selected from the group consisting of an inhibitor of activity or production of a bioactive substance selected from the group consisting of TGF-α, HGF, PDGF, VEGF, IGF, MMP, FGF, uPA, cathepsin, and SDF-1, a cell activity suppressor, a growth inhibitor, an apoptosis inducer, and an siRNA, ribozyme, antisense nucleic acid, DNA/RNA chimeric polynucleotide, or vector expressing same that targets one or more molecules from among an extracellular matrix constituent molecule produced by cancer-associated fibroblasts and a molecule involved in the production or secretion of the extracellular matrix constituent molecule.

(47) The composition according to (46), wherein the molecule involved in the production or secretion of the extracellular matrix constituent molecule is HSP47.

(48) The composition according to any one of (42) to (44), wherein the drug and the targeting agent are mixed at a place of medical treatment or in the vicinity thereof.

(49) The composition according to any one of (45) to (47), wherein the drug and the carrier are mixed at a place of medical treatment or in the vicinity thereof.

(50) A preparation kit for the composition according to any one of (42) to (49), wherein it comprises one or more containers comprising singly or in combination the drug, the targeting agent, and as necessary carrier constituent substances other than the targeting agent.

### Effects of the Invention

The carrier of the present invention specifically targets a cancer cell and a CAF, and efficiently delivers to a cancer cell and/or a CAF a desired substance or body such as, for example, a drug that controls the activity or growth of a cancer cell or a CAF, thus enabling a desired effect such as, for example, suppression of the activity or growth of a cancer cell or a CAF thereby curing cancer, suppressing the advance thereof, and preventing the onset thereof, to be achieved with the highest efficiency and the minimum side effects.

Since the anticancer composition of the present invention is based on the completely novel approach of treating a cancer by acting on a CAF in addition to a cancer cell itself, efficacy can be expected on cancers for which a conventional treatment method could not give satisfactory results and, furthermore, a synergistic effect due to combined use with a conventional anticancer agent, angiogenesis inhibitor, etc. can be anticipated.

Furthermore, since the carrier of the present invention can specifically deliver a substance to a cancer cell and a CAF, it can be utilized for specifically labeling a cancer cell and a CAF, gene transfer, etc.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a photographic diagram of cancer tissue-derived cells immunostained with respect to α-SMA, vimentin, and desmin.

FIG. 2 is a graph showing change in the number of cancer cells when cancer cells and CAFs or normal fibroblasts are cocultured.

FIG. 3 is a graph in which the percentage introduction of siRNA to CAFs or normal fibroblasts when siRNA is delivered by various liposomes is compared over time.

FIG. 4 is a photographic diagram showing the localization of siRNA in CAFs that have been reacted with VA-lip-siRNA or lip-siRNA.

FIG. 5 is a photographic diagram showing the localization of siRNA in CAFs and normal fibroblasts that have been reacted with VA-lip-siRNA, lip-siRNA, or VA-lip.

FIG. 6 is a photographic diagram showing the localization of DNR in CAFs that have been reacted with VA-lip-DNR or lip-DNR. The numbers in the diagram show elapsed time (min) from the start of the reaction. The magnification is 200 times (400 times for enlarged images).

FIG. 7 is a photographic diagram showing the localization of DNR in normal fibroblasts that have been reacted with VA-lip-DNR or lip-DNR. The numbers in the diagram show elapsed time (min) from the start of the reaction. The magnification is 200 times.

FIG. 8 is a photographic diagram showing the localization of DNR in skin fibroblasts that have been reacted with VA-lip-DNR or lip-DNR. The numbers in the diagram show elapsed time (min) from the start of the reaction. The magnification is 200 times.

FIG. 9 is a photographic diagram showing the localization of DNR in CAFs that have been reacted with VA-lip-DNR (left) or lip-DNR (right). The magnification is 400 times (800 times for enlarged image).

FIG. 10 is a photographic diagram showing daunorubicin emitting red fluorescence under green excitation light (upper left), DAPI (4′,6-diamino-2-phenylindole) emitting blue fluorescence under UV excitation light (upper right), and a merged image exhibiting a purple color (lower).

FIG. 11 is a photographic diagram showing the localization of DNR in CAFs that have been either not treated (No treatment: upper left) or reacted with DaunoXome® (VA−: upper right), DaunoXome®+retinol (VA+: lower left), or DaunoXome®+retinoic acid (Retinoic acid+: lower right). The magnification is 400 times.

FIG. 12 is a photographic diagram showing the localization of DNR in HT-1080 that has been either not treated (No treatment: upper left) or reacted with DaunoXome® (VA−: upper right), DaunoXome®+retinol (VA+: lower left), or DaunoXome®+retinoic acid (Retinoic acid+: lower right). The magnification is 400 times.

FIG. 13 is a photographic diagram showing the localization of DNR in HepG2 that has been either not treated (No treatment: upper left) or reacted with DaunoXome® (VA−: upper right), DaunoXome®+retinol (VA+: lower left), or DaunoXome®+retinoic acid (Retinoic acid+: lower right). The magnification is 400 times.

FIG. 14 is a graph showing the result of evaluation of the growth inhibitory activity of VA-lip-siRNA toward CAFs or normal fibroblasts. The ordinate denotes the percentage viable cell count after treatment when the viable cell count prior to treatment is 100.

FIG. 15 is a graph showing the result of evaluation of the growth inhibitory activity of VA-lip-DNR toward CAFs or normal fibroblasts. The ordinate denotes the percentage viable cell count after treatment when the viable cell count prior to treatment is 100.

FIG. 16 is a photographic diagram showing the intracellular localization state of liposomal DNR (VA−) or VA-bound liposomal DNR (VA+) in the human fibrosarcoma-derived cell line HT-1080. The upper section shows the localization of DNR, the lower section shows cells that have been subjected to nuclear staining with DAPI, and the figures show the time after addition.

FIG. 17 is a photographic diagram showing the intracellular localization state of liposomal DNR (VA−) or VA-bound liposomal DNR (VA+) in the human fibrosarcoma-derived cell line HS913T. The upper section shows the localization of DNR, the lower section shows cells that have been subjected to nuclear staining with DAPI, and the figures show the time after addition.

FIG. 18 is a photographic diagram showing the intracellular localization state of liposomal DNR (VA−) or VA-bound liposomal DNR (VA+) in the human fibrosarcoma-derived cell line Sw684. The upper section shows the localization of DNR, the lower section shows cells that have been subjected to nuclear staining with DAPI, and the figures show the time after addition.

FIG. 19 is a photographic diagram showing the intracellular localization state of liposomal DNR. (VA (−)) or VA-bound liposomal DNR (VA (+)) in HT-1080, HS913T, Sw684, Huh7, MCF₇, and Saos2 cells (15 min after addition). Blank denotes a microscopic image when neither liposomal DNR or VA-bound liposomal DNR were added.

FIG. 20 is a graph of the evaluation of the growth-inhibitory activity of liposomal DNR or VA-bound liposomal DNR toward the human fibrosarcoma-derived cell lines HT-1080, HS913T, and Sw684.

FIG. 21 is a photographic diagram showing the localization of siRNA in the tumor tissue of a tumor-bearing mouse to which VA-lip-siRNA or lip-siRNA had been intravenously administered. The right-hand side shows an individual to which VA-lip-siRNA had been administered, the left-hand side shows an individual to which lip-siRNA had been administered, the top shows an FAM image, and the bottom shows a merged FAM and Cy3 image. The magnification is 200 times.

FIG. 22 is a photographic diagram showing the localization of siRNA in the tumor tissue of a tumor-bearing mouse to which VA-lip-siRNA had been intravenously administered. The upper left shows an FAM image, the upper right shows a Cy3 image, the lower left shows a merged FAM and Cy3 image, and the lower right shows a merged FAM, Cy3, and DAPI image. The magnification is 200 times.

FIG. 23 is a graph showing the results of evaluating the in vivo antitumor activity of VA-lip-DNR (administered twice a week). The ordinate denotes the tumor mass volume (mm³), and the abscissa denotes the number of days after starting the treatment. FIG. 24 VA-conjugate addition to liposomes via decoration enhances siRNA activity

FIG. 25 VA-conjugate addition to liposomes via co-solubilization enhances siRNA activity

FIG. 26 VA-conjugate addition to liposomes via co-solubilization enhances siRNA activity

FIG. 27 VA-conjugate addition to lipoplexes via co-solubilization enhance siRNA activity

FIG. 28 VA-conjugate addition to lipoplexes via co-solubilization vs. decoration.

FIG. 29 is a diagram showing the efficacy of diVA-lip-siRNA-DY647 delivery to cancer cells A549, PANC-1 and HepG2. The mean fluorescence intensity (MFI) of A549 (FIG. 29A), PANC-1 (FIG. 29B) and HepG2 (FIG. 29C) cells treated with diVA-lip-siRNA-DY647 or lip-siRNA-DY647 is indicated respectively.

### DETAILED DESCRIPTION OF THE PREFERRED EMBODIMENTS

The present invention is explained in detail below.

In the present invention, the cancer cell is not particularly limited, and examples thereof include a cancer cell in sarcomas such as fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, and osteosarcoma, any kind of cancer such as brain tumor, head and neck carcinoma, breast carcinoma, lung carcinoma, esophageal carcinoma, stomach carcinoma, duodenal carcinoma, appendiceal carcinoma, colon carcinoma, rectal carcinoma, hepatic carcinoma, pancreatic carcinoma, gallbladder carcinoma, bile duct carcinoma, anal carcinoma, kidney carcinoma, ureteral carcinoma, bladder carcinoma, prostate carcinoma, penile carcinoma, testicular carcinoma, uterine carcinoma, ovarian carcinoma, vulvar carcinoma, vaginal carcinoma, and skin carcinoma and, furthermore, leukemia, malignant lymphoma, etc. In the present invention, ‘cancer’ includes carcinoma and sarcoma. The cancer cell in the present invention is therefore present at any site such as, for example, the brain, head and neck, breast, limbs, lung, heart, thymus, esophagus, stomach, small intestine (duodenum, jejunum, ileum), large intestine (colon, cecum, appendix, rectum), liver, pancreas, gallbladder, anus, kidney, ureter, bladder, prostate, penis, testis, uterus, ovary, vulva, vagina, skin, striated muscle, smooth muscle, synovial membrane, cartilage, bone, thyroid, adrenal gland, peritoneum, mesentery, bone marrow, blood, vascular system, lymphatic system such as lymph nodes, lymphatic fluid, etc.

In one embodiment of the present invention, a cancer cell is preferably present at sites other than the liver and pancreas. Therefore, in this embodiment, the cancer cell is preferably present in, for example, the brain, head and neck, breast, limbs, lung, heart, thymus, esophagus, stomach, small intestine (duodenum, jejunum, ileum), large intestine (colon, cecum, appendix, rectum), gallbladder, anus, kidney, ureter, bladder, prostate, penis, testis, uterus, ovary, vulva, vagina, skin, striated muscle, smooth muscle, synovial membrane, cartilage, bone, thyroid, adrenal gland, peritoneum, mesentery, bone marrow, blood, vascular system, lymphatic system such as lymph nodes, lymphatic fluid, etc. Furthermore, in one embodiment of the present invention, a cancer cell is preferably that other than a hepatic carcinoma cell and a pancreatic carcinoma cell.

In the present invention, a cancer-associated fibroblast (CAF) means an α-SMA (smooth muscle actin) positive fibroblast present in the interior and/or the periphery of a cancer lesion. The presence of a CAF is confirmed with respect to various cancers such as colon carcinoma, lung carcinoma, prostate carcinoma, breast carcinoma, stomach carcinoma, bile duct carcinoma, and basal cell carcinoma.

In the present invention, whether or not given cell is CAF is determined by the following method. That is, a cell present in the interior and/or the periphery of the cancer lesion is immunostained with a labeled antibody for α-SMA, which is a CAF marker, for example, FITC-labeled anti α-SMA antibody or Cy3-labeled anti α-SMA antibody, and that detected by α-SMA is determined to be a CAF.

Cancer accompanied by CAF in the present invention is not particularly limited, and examples thereof include solid carcinomas such as brain tumor, head and neck carcinoma, breast carcinoma, lung carcinoma, esophageal carcinoma, stomach carcinoma, duodenal carcinoma, appendiceal carcinoma, colon carcinoma, rectal carcinoma, hepatic carcinoma, pancreatic carcinoma, gallbladder carcinoma, bile duct carcinoma, anal carcinoma, kidney carcinoma, ureteral carcinoma, bladder carcinoma, prostate carcinoma, penile carcinoma, testicular carcinoma, uterine carcinoma, ovarian carcinoma, vulvar carcinoma, vaginal carcinoma, and skin carcinoma. Furthermore, a CAF typically accompanies a carcinoma, but as long as similar properties are possessed, it may accompany a malignant solid tumor other than a carcinoma, for example, a sarcoma such as fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, or osteosarcoma, and they are included in the scope of the present invention.

In one embodiment of the present invention, a CAF is preferably present at sites other than the liver and pancreas. Therefore, in this embodiment, the CAF is present in, for example, the brain, head and neck, breast, limbs, lung, heart, thymus, esophagus, stomach, small intestine (duodenum, jejunum, ileum), large intestine (colon, cecum, appendix, rectum), gallbladder, anus, kidney, ureter, bladder, prostate, penis, testis, uterus, ovary, vulva, vagina, skin, striated muscle, smooth muscle, synovial membrane, cartilage, bone, thyroid, adrenal gland, peritoneum, mesentery, etc.

A retinoid is a member of the class of compounds having a skeleton in which four isoprenoid units are bonded in a head-to-tail manner. See G. P. Moss, “Biochemical Nomenclature and Related Documents,” 2nd Ed. Portland Press, pp. 247-251 (1992). Vitamin A is a generic descriptor for a retinoid qualitatively showing the biological activity of retinol. Retinoid in the present invention promotes specific substance delivery to a cancer cell and a CAF (that is, the substance is targeted at these cells). Such a retinoid is not particularly limited, and examples thereof include retinoid derivatives such as retinol, retinal, retinoic acid, an ester of retinol and a fatty acid, an ester of an aliphatic alcohol and retinoic acid, etretinate, tretinoin, isotretinoin, adapalene, acitretine, tazarotene, and retinol palmitate, and vitamin A analogues such as fenretinide (4-HPR), and bexarotene.

In the present invention, retinoid has the same meaning as retinoid derivative and/or vitamin A analogue. Although the mechanism by which a retinoid promotes specific substance delivery to a cancer cell and a CAF has not been completely elucidated, it is surmised that uptake via a certain type of receptor on the surface of a cancer cell and a CAF is involved.

Among them, retinol, retinal, retinoic acid, an ester of retinol and a fatty acid (e.g. retinyl acetate, retinyl palmitate, retinyl stearate, and retinyl laurate) and an ester of an aliphatic alcohol and retinoic acid (e.g. ethyl retinoate) are preferable from the viewpoint of efficiency of specific delivery of a substance to a cancer cell and a CAF.

All retinoid isomers, such as cis-trans, are included in the scope of the present invention. The retinoid may be substituted with one or more substituents. The retinoid in the present invention includes a retinoid in an isolated state as well as in a solution or mixture state with a medium that can dissolve or retain the retinoid.

One aspect of the present invention relates to a targeting agent comprising a retinoid, to a cell selected from the group consisting of a cancer cell and a cancer-associated fibroblast. The targeting referred to here means enabling a substance such as a drug or a drug carrier to be delivered to a specific target such as a specific cell or tissue (in the present invention a cell selected from the group consisting of a cancer cell and a cancer-associated fibroblasts) more rapidly, efficiently, and/or in a larger quantity than with non-target cell or tissue and a substance that is non-targeted, that is, it enables specific delivery to a target, and the targeting agent means a substance that can subject a substance to the above-mentioned targeting when it binds to or reacts with the substance. Therefore, in the present specification, for example, ‘cancer cell-specific carrier or composition’ has the same meaning as ‘cancer cell-targeted carrier or composition’. When the targeting agent is in the configuration of a molecule, this has the same meaning as a targeting molecule.

Another aspect of the present invention relates to a targeting agent comprising a compound for facilitating drug delivery to a target cell, consisting of the structure (targeting molecule)ₘ-linker-(targeting molecule)ₙ, wherein the targeting molecule is a retinoid or a fat soluble vitamin having a specific receptor (or activation/binding site) on the target cell; and wherein m and n are independently 0, 1, 2, or 3 (except that m and n are not both zero); and wherein the linker comprises a polyethylene glycol (PEG) or PEG-like molecule and is designated “Formula A”.

The invention also includes a compound for facilitating drug delivery to a target cell, consisting of the structure (lipid)ₘ-linker-(targeting molecule)ₙ, wherein the targeting molecule is a retinoid or a fat soluble vitamin having a specific receptor on the target cell; wherein m and n are independently 0, 1, 2, or 3 (except that m and n are not both zero); and wherein the linker comprises a polyethylene glycol (PEG) PEG-like molecule and is designated “Formula B”.

It has now been discovered that the compounds of Formula A or Formula B impart properties to the formulations of the invention not previously seen. Formulations of the invention that include compounds of Formula A or Formula B result in superior reduction in gene expression, as compared to formulations that do not include these compounds. Particularly surprising is the ability of formulations of the invention that include compounds of Formula A to reduce the expression of HSP47.

In certain preferred embodiments, the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

Preferred embodiments include compounds where the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH. In other embodiments, the PEG is mono-disperse.

Another embodiment provides a compound where Formula A is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

In another preferred embodiment, the compound is of the formula

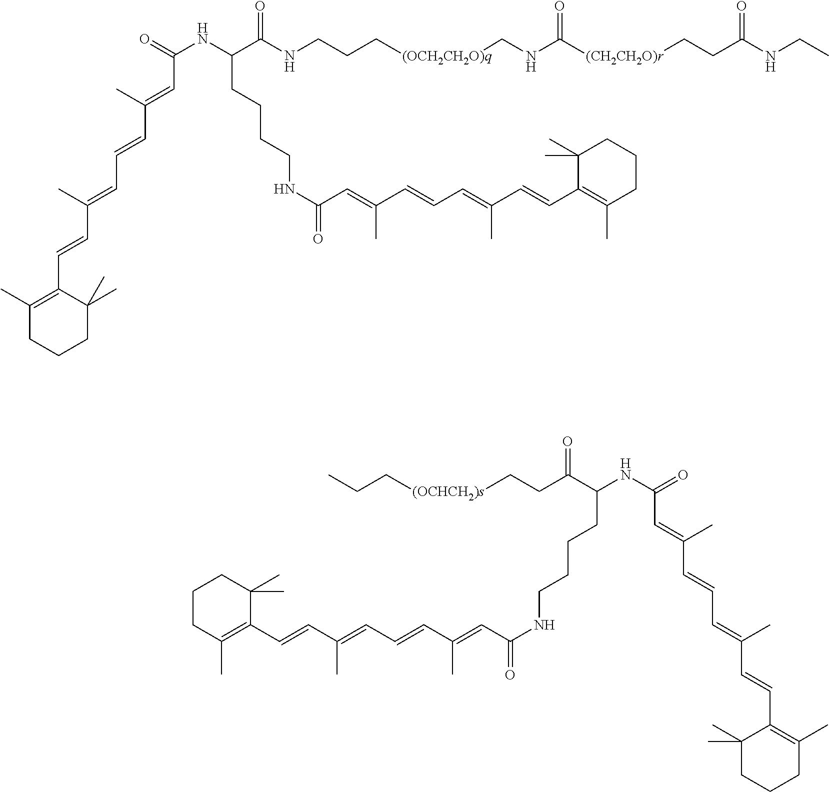

wherein q, r, and s are each independently 1, 2, 3, 4, 5, 6, 7, 8, 9, or 10.

In other preferred embodiments, q, r and s are 3, 5 and 3, respectively, and the formula of the compound comprises

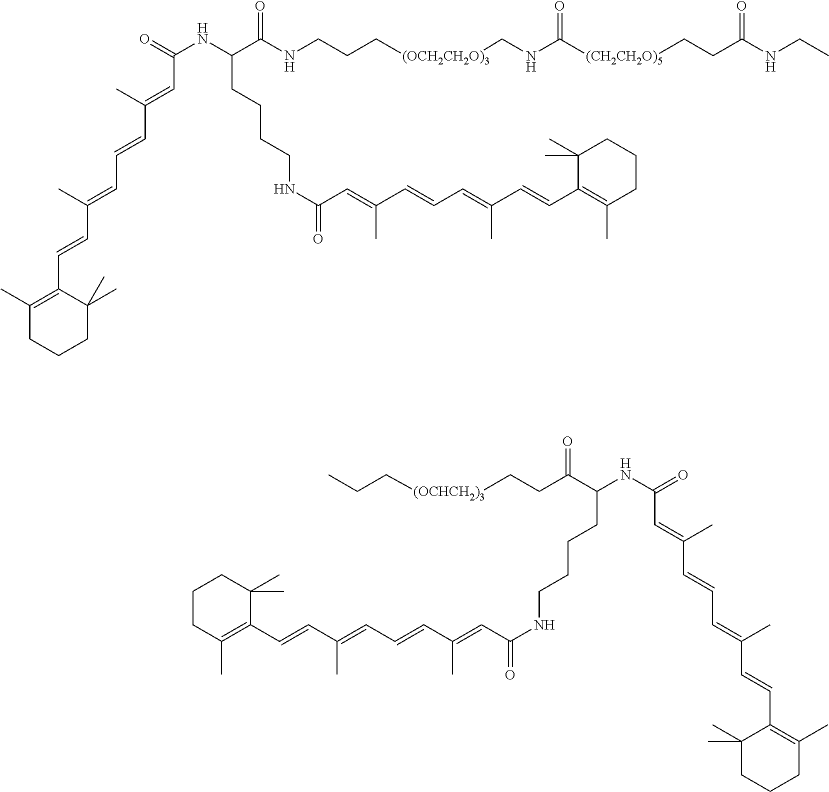

Other embodiments of the invention include the structures shown in Table 1.

TABLE 1

| Lipid Name | Compound Structure |
|---|---|
| SatDiVA | 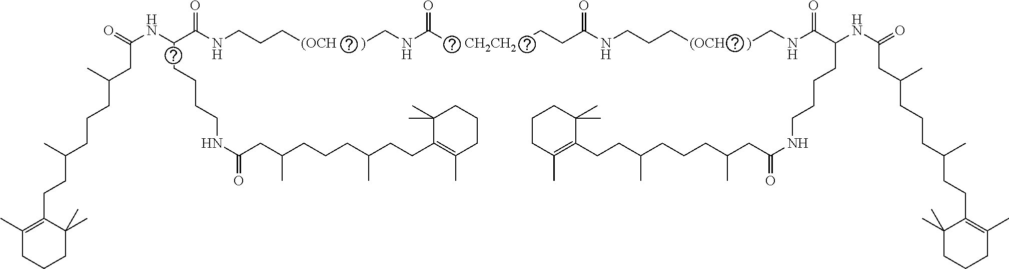 |
| SimDiVa | 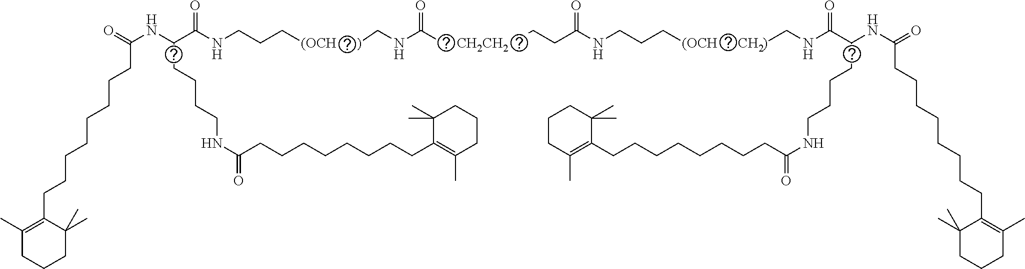 |
| DiVA-PEG18 | 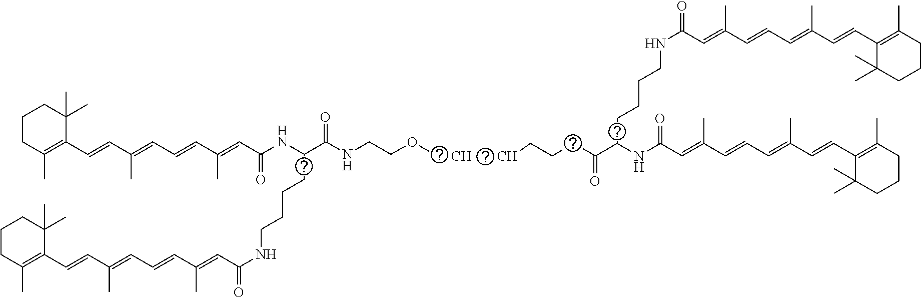 |
| TriVA | 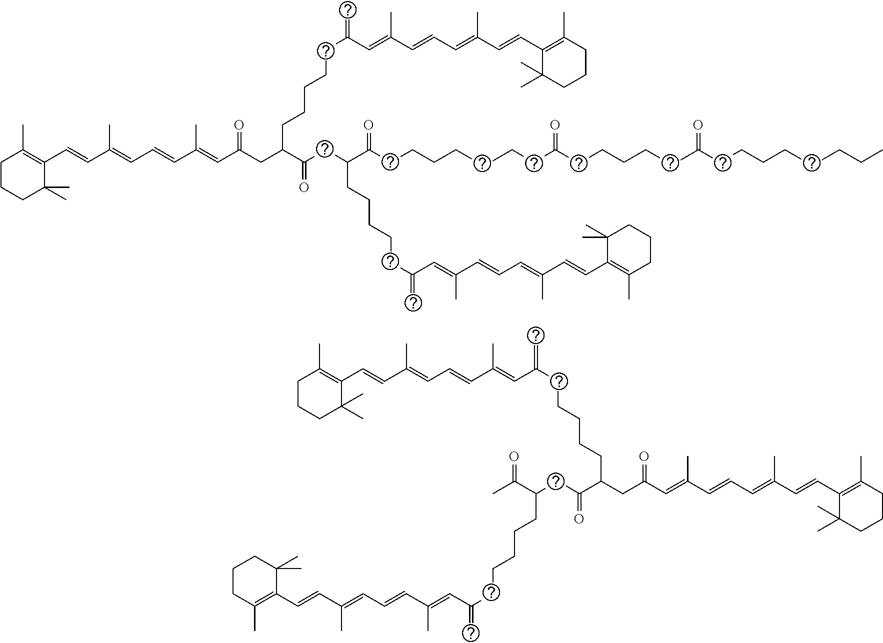 |
| 4TTNPB | 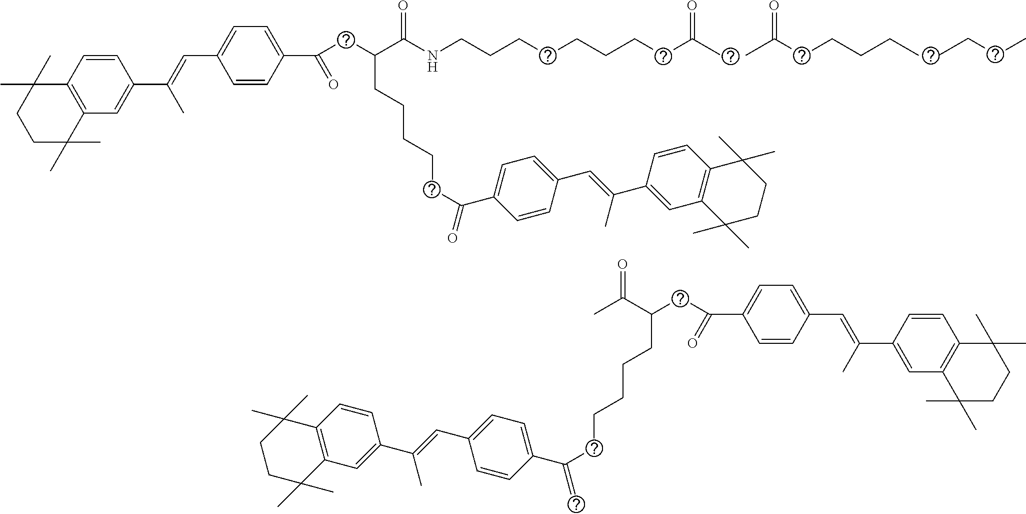 |
| 4Myr | 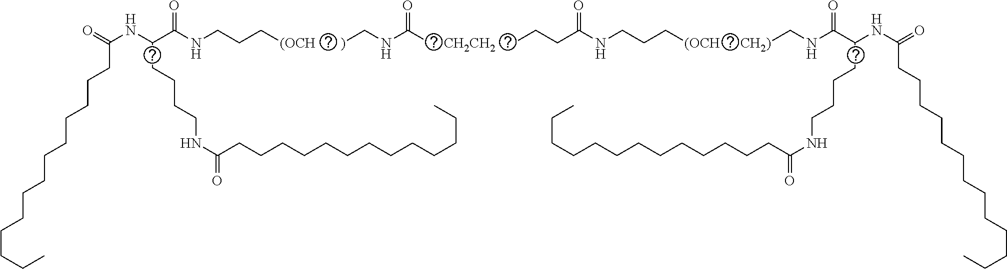 |
| DiVA-242 | 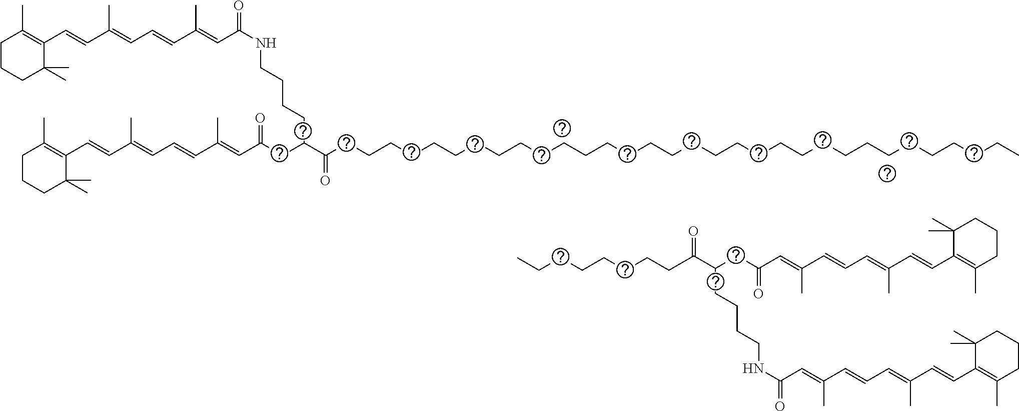 |

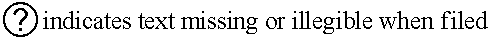

Also within the scope of the invention are formulations comprising at least one compound of Formula A or B and siRNA. It is envisioned that any siRNA molecule can be used within the scope of the invention. Examples of siRNA include:

(SEQ. ID. NO. 9)
Sense (5′->3′) GGACAGGCCUCUACAACUATT
(SEQ. ID. NO. 10)
Antisense (3′->5′) TTCCUGUCCGGAGAUGUUGAU
and
(SEQ. ID. NO. 11)
Sense (5′->3′) GGACAGGCCUGUACAACUATT
(SEQ. ID. NO. 12)
Antisense (3′->5′) TTCCUGUCCGGACAUGUUGAU

The targeting agent of the present invention may be formed from the above-mentioned compounds on its own or may include a constituent element other than the compounds, for example, an element for promoting or stabilizing binding between the targeting agent and a carrier or a drug, an element for protecting the retinoid during storage, during use in a production of a formulation, or during storage of a formulation, or a spacer for spatially separating the compounds from a carrier or a drug. The targeting agent of the present invention is bound to any carrier or drug, and can target this carrier or drug at a cell selected from the group consisting of a cancer cell and a cancer-associated fibroblast.

Furthermore, the present invention relates to a substance delivery carrier to a cell selected from the group consisting of a cancer cell and a cancer-associated fibroblast, the carrier including the targeting agent. The carrier of the present invention may be formed from the targeting agent on its own or may be formed by making the targeting agent bind to or be enclosed in another constituent component, other than the targeting agent, of the carrier. Therefore, the carrier of the present invention may include a constituent component other than the targeting agent. Such a component is not particularly limited, and any component known in the medicinal and pharmaceutical fields may be used, but those that can enclose the targeting agent, and the retinoid in particular, or can bind thereto are preferable.

Other embodiments include a drug carrier comprising a liposomal composition. The liposomal composition can comprise a lipid vesicle comprising a bilayer of lipid molecules. In certain embodiments it may preferred that the retinoid and/or derivative thereof is at least partially exposed on the exterior of the drug carrier before the drug carrier reaches the stellate cell.

Certain embodiments of the present invention provide that the lipid molecules comprise one or more lipids selected from the group consisting of HEDC, DODC, HEDODC, DSPE, DOPE, and DC-6-14. In other embodiments, the lipid molecules can further comprise S104.

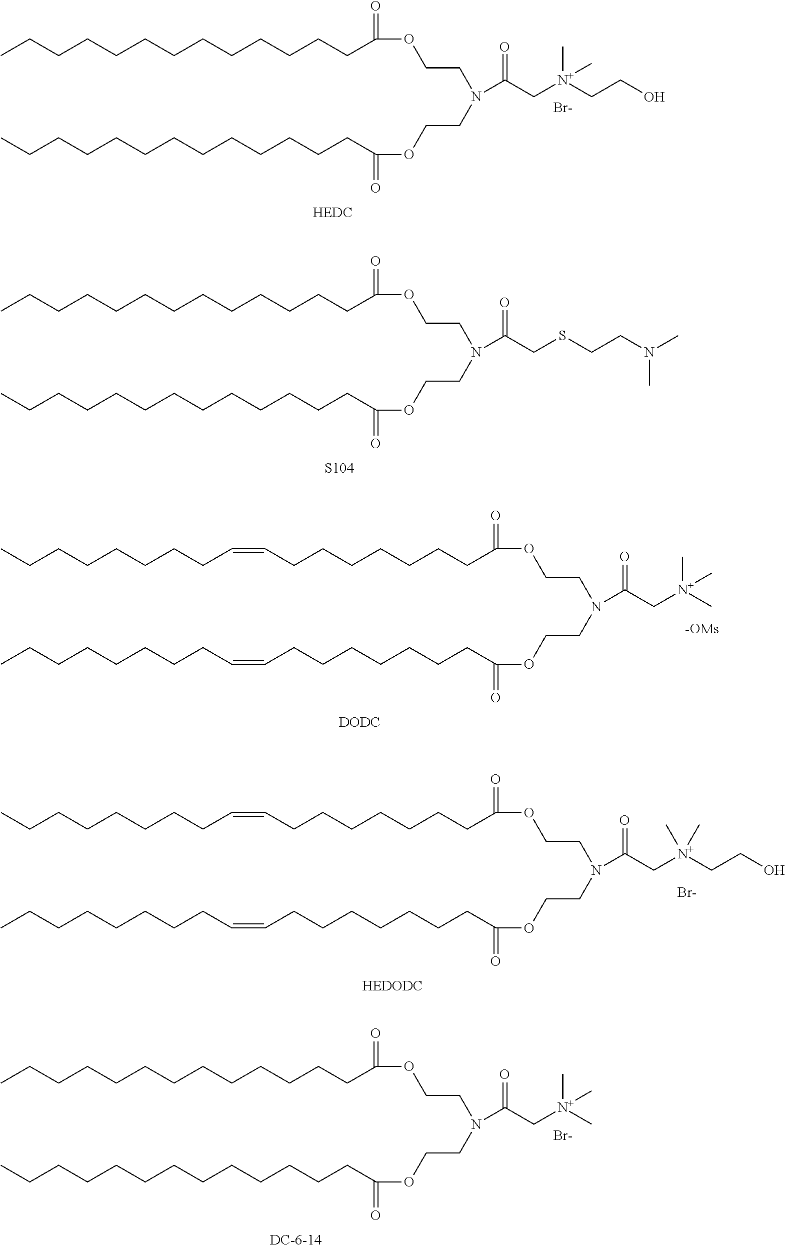

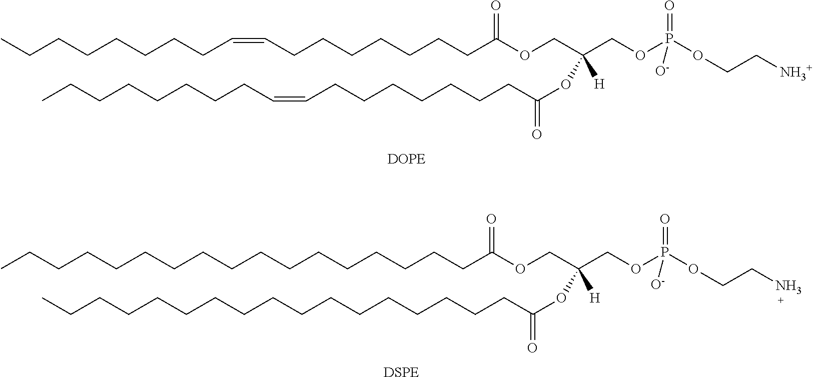 [SMILES: CCCCCCCC/C=C\CCCCCCCC(=O)OC[C@H](COP(=O)([O-])OCC[NH3+])OC(=O)CCCCCCC/C=C\CCCCCCCC ; "DOPE" ; CCCCCCCCCCCCCCCCCC(=O)OC[C@H](COP(=O)([O-])OCCN)OC(=O)CCCCCCCCCCCCCCCCC ; "DSPE"]

In some embodiments, the siRNA will be encapsulated by the liposome so that the siRNA is inaccessible to the aqueous medium. In other embodiments, the siRNA will not be encapsulated by the liposome. In such embodiments, the siRNA can be complexed on the outer surface of the liposome. In these embodiments, the siRNA is accessible to the aqueous medium.

Other embodiments include a drug carrier comprising a liposomal composition. The liposomal composition can comprise a lipid vesicle comprising a bilayer of lipid molecules. In other embodiments, the retinoid and/or derivative thereof is at least partially exposed on the exterior of the drug carrier before the drug carrier reaches the stellate cell.

In certain preferred embodiments, the retinoid and/or derivative thereof is 0.1 mol % to 20 mol % of the lipid molecules.

The forgoing compositions can also include PEG-conjugated lipids, which are known in the art per se, including PEG-phospholipids and PEG-ceramides, including one or more molecules selected from the following: PEG2000-DSPE, PEG2000-DPPE, PEG2000-DMPE, PEG2000-DOPE, PEG1000-D SPE, PEG1000-DPPE, PEG1000-DMPE, PEG1000-DOPE, PEG550-DSPE, PEG550-DPPE, PEG-550DMPE, PEG-1000DOPE, PEG-cholesterol, PEG2000-ceramide, PEG1000-ceramide, PEG750-ceramide, and PEG550-ceramide.

The foregoing compositions of the invention can include one or more phospholipids such as, for example, 1,2-distearoyl-sn-glycero-3-phosphocholine (“DSPC”), dipalmitoylphosphatidylcholine (“DPPC”), 1,2-dipalmitoyl-sn-glycero-3-phosphoethanolamine (“DPPE”), and 1,2-dioleoyl-sn-glycero-3-phosphoethanolamine (“DOPE”). Preferably, the helper phospholipid is DOPE.

Examples of such a component include a lipid, for example, a phospholipid such as glycerophospholipid, a sphingolipid such as sphingomyelin, a sterol such as cholesterol, a vegetable oil such as soybean oil or poppy seed oil, a mineral oil, and a lecithin such as egg-yolk lecithin, but the examples are not limited thereto. Among them, those that can form a liposome are preferable, for example, a natural phospholipid such as lecithin, a semisynthetic phospholipid such as dimyristoylphosphatidylcholine (DMPC), dipalmitoylphosphatidylcholine (DPPC), or distearoylphosphatidylcholine (DSPC), dioleylphosphatidylethanolamine (DOPE), dilauroylphosphatidylcholin (DLPC), and cholesterol.

A particularly preferred component is a component that can avoid capture by the reticuloendothelial system, and examples thereof include cationic lipids such as N-(α-trimethylammonioacetyl)-didodecyl-D-glutamate chloride (TMAG), N,N′,N″,N′″-tetramethyl-N,N′,N″,N′″-tetrapalmitylspermine (TMTPS),2,3-dioleyloxy-N-[2(sperminecarboxamido)ethyl]-N,N-dimethyl-1-propanaminium trifluoroacetate (DOSPA), N-[1-(2,3-dioleyloxy)propyl]-N,N,N-trimethylammonium chloride (DOTMA), dioctadecyldimethylammonium chloride (DODAC), didodecylammonium bromide (DDAB), 1,2-dioleyloxy-3-trimethylammoniopropane (DOTAP), 3β-[N—(N′,N′-dimethylaminoethane)carbamoyl]cholesterol (DC-Chol), 1,2-dimyristoyloxypropyl-3-dimethylhydroxyethylammonium bromide (DMRIE), and O,O′-ditetradecanoyl-N-α-trimethylammonioacetyl)diethanolamine chloride (DC-6-14).

The binding of the targeting agent to the carrier of the present invention or the enclosing of it therein is also possible by binding or enclosing the targeting agent to or in a constituent component, other than the targeting agent, of the carrier by a chemical and/or physical method. Alternatively, the binding or enclosing the targeting agent to or in the carrier of the present invention can also be carried out by mixing the targeting agent and a constituent component, other than the targeting agent, of the carrier when preparing the carrier. The amount of targeting agent bound to or enclosed in the carrier of the present invention may be, as a weight ratio in the carrier constituent components including the targeting agent, 0.01% to 100%, preferably 0.2% to 20%, and more preferably 1% to 5%. The binding or enclosing of the targeting agent to or in the carrier may be carried out before a drug, etc. is supported on the carrier, may be carried out at the same time as mixing the carrier, the targeting agent, and a drug, etc., or may be carried out by mixing the targeting agent with a carrier on which a drug, etc. is already supported. Therefore, the present invention also relates to a process for producing a formulation targeted at a cell selected from the group consisting of a cancer cell and a CAF, the process including a step of binding a targeting agent to any existing drug binding carrier or drug encapsulating carrier, for example, a liposomal formulation such as DaunoXome®, Doxil, Caelyx®, or Myocet®.

The configuration of the carrier of the present invention may be any configuration as long as a desired substance or body can be carried to a target cancer cell or CAF, and although not limited thereto, examples thereof include a macromolecular micelle, a liposome, an emulsion, microspheres, and nanospheres. The size of the carrier of the present invention can be changed according to the type, etc. of drug. Such a size is not particularly limited and, for example, the diameter is preferably 50 to 200 μm, and more preferably 75 to 150 μm. This is because such a size is suitable for exhibiting the EPR effect which promotes the accumulation in cancer tissue, and is also suitable for delivery of a drug that controls the activity or growth of a cancer cell and/or a CAF, which is described later. Such a diameter is measured by a dynamic light scattering method.

In the carrier of the present invention, the molar ratio (abundance ratio) of the targeting agent to constituent components, other than the targeting agent, of the carrier when administered is preferably 8:1 to 1:4, more preferably 4:1 to 1:2, yet more preferably 3:1 to 1:1, and particularly preferably 2:1. Without being bound by theory, it is believed that such a molar ratio is effective in giving good binding or enclosing of the targeting agent to or in a carrier (that is, the targeting function of the targeting agent is not impaired) and in specifically delivering a substance to a cancer cell or a CAF.

In the present invention, from the viewpoint of high delivery efficiency, wide selection of substances to be delivered, ease of making a formulation, etc., a liposomal configuration is preferable among the configurations, and a cationic liposome that includes a cationic lipid is particularly preferable.

The carrier of the present invention may contain a substance to be carried within its interior, may be attached to the exterior of a substance to be carried, or may be mixed with a substance to be carried, as long as the targeting agent contained therein is present in such a configuration that it can exhibit a targeting function. The ‘exhibiting a targeting function’ referred to here means that the carrier containing the targeting agent reaches and/or is taken up by the target cancer cell and/or CAF more rapidly, efficiently and/or in a larger quantity than with a carrier not containing the targeting agent, and this may easily be confirmed by, for example, adding a labeled or label-containing carrier to cultured cancer cell and/or CAF, and analyzing sites where the label is present after a predetermined period of time. Unpredictably, the present inventors have found that specific substance delivery to a cancer cell and/or a CAF is efficiently realized by at least partially exposing the targeting agent on the exterior of a formulation containing the carrier at the latest by the time it reaches the cancer cell and/or CAF. The present inventors consider this to be a phenomenon in which the targeting agent exposed on the exterior of the formulation containing the carrier is taken up by the cancer cell and/or CAF more efficiently than by normal diffusion, via a certain type of receptor on the surface of the cancer cell and/or CAF. A technique for exposing the targeting agent on the exterior of the formulation containing the carrier is not particularly limited; for example, when preparing a carrier, excess targeting agent may be added relative to constituent components, other than the targeting agent, of the carrier. More specifically, in order to efficiently expose the targeting agent on the exterior of a formulation containing the carrier, the molar ratio (compounding ratio) of the targeting agent to constituent components, other than the targeting agent, of the carrier when compounded is preferably 8:1 to 1:4, more preferably 4:1 to 1:2, yet more preferably 3:1 to 1:1, and particularly preferably 2:1.

The substance or body that is delivered by the present carrier is not particularly limited, and it preferably has a size such that it can physically move within the body of a living being from an administration site to a lesion site where a cancer cell and/or a CAF is/are present. Therefore, the carrier of the present invention can carry not only a substance such as an atom, a molecule, a compound, a protein, or a nucleic acid, but also a body such as a vector, a virus particle, a cell, a drug-releasing system formed from one or more elements, or a micromachine. The above substance or body preferably has the property of having some influence on a cancer cell and/or a CAF, and examples thereof include those that label a cancer cell and/or a CAF and those that control (e.g. increase or suppress) the activity and growth of a cancer cell and/or a CAF.

Therefore, in one embodiment of the present invention, the substance that the carrier delivers is ‘a drug controlling the activity or growth of a cancer cell and/or a CAF’. The activity of a cancer cell referred to here indicates various activities such as secretion, uptake, migration, etc. exhibited by a cancer cell, and in the present invention among them it typically means, in particular, activities involved in the onset, progression, recurrence and/or metastasis of a cancer, and the manifestation, exacerbation, etc. of symptoms such as cachexia. Examples of such activities include, but are not limited to, the production/secretion of parathyroid hormone-related protein (PTHrP), immunosuppressive acidic protein (IAP), etc.

Furthermore, the activity of a CAF means various activities such as secretion, uptake, migration, etc. exhibited by CAF, and in the present invention it typically means activities involved in the onset and/or progression of a cancer in particular. Examples of such activities include the production/secretion of bioactive substances such as TGF-β, HGF, PDGF, VEGF, IGF (IFG1, IGF2, etc.), MMP (MMP1, 2, 3, 9, 11, 13, 14, etc.), FGF (FGF7, bFGF, etc.), uPA, cathepsin, and SDF-1, and extracellular matrix components such as collagen, proteoglycan, tenascin, fibronectin, thrombospondin, osteopontin, osteonectin, and elastin.

Therefore, the drug controlling the activity and growth of a cancer cell may be any drug that directly or indirectly suppresses the physical, chemical, and/or physiological actions, etc. of a cancer cell related to the onset, progression, and/or recurrence of a cancer, and while not being limited thereto, it includes anticancer agents that suppress the onset, progression, and/or recurrence of a cancer, and examples thereof include, but are not limited to, alkylating agents such as ifosfamide, nimustine hydrochloride, cyclophosphamide, dacarbazine, melphalan, and ranimustine, antimetabolites such as gemcitabine hydrochloride, enocitabine, cytarabine ocfosfate, a cytarabine formulation, tegafur/uracil, a tegafur/gimeracil/oteracil potassium mixture, doxifluridine, hydroxycarbamide, fluorouracil, methotrexate, and mercaptopurine, antitumor antibiotics such as idarubicin hydrochloride, epirubicin hydrochloride, daunorubicin hydrochloride, daunorubicin citrate, doxorubicin hydrochloride, pirarubicin hydrochloride, bleomycin hydrochloride, peplomycin sulfate, mitoxantrone hydrochloride, and mitomycin C, alkaloids such as etoposide, irinotecan hydrochloride, vinorelbine tartrate, docetaxel hydrate, paclitaxel, vincristine sulfate, vindesine sulfate, and vinblastine sulfate, hormone therapy agents such as anastrozole, tamoxifen citrate, toremifene citrate, bicalutamide, flutamide; and estramustine phosphate, platinum complexes such as carboplatin, cisplatin, and nedaplatin, angiogenesis inhibitors such as thalidomide, neovastat, and bevacizumab, L-asparaginase etc., drugs inhibiting the activity or production of the above bioactive substances, such as, for example, antibodies and antibody fragments that neutralize the above bioactive substances, and substances that suppress expression of the above bioactive substances, such as an siRNA, a ribozyme, an antisense nucleic acid (including RNA, DNA, PNA, and a composite thereof), substances that have a dominant negative effect such as a dominant negative mutant, vectors expressing same, cell activity inhibitors such as a sodium channel inhibitor, cell-growth inhibitors, and apoptosis inducers such as compound 861 and gliotoxin. Furthermore, the ‘drug controlling the activity or growth of a cancer cell’ in the present invention may be any drug that directly or indirectly promotes the physical, chemical, and/or physiological actions, etc. of a cancer cell directly or indirectly related to suppressing the onset, progression, and/or recurrence of a cancer. Among the above-mentioned drugs, an anticancer agent is particularly preferable from the viewpoint of therapeutic effect, etc.

Moreover, the ‘drug controlling the activity or growth of a CAF’ referred to here may be any drug that directly or indirectly suppresses the physical, chemical, and/or physiological actions, etc. of a CAF related to the onset and/or progression of a cancer, and examples thereof include, without being limited thereto, drugs that inhibit the activity or production of the above bioactive substances, for example, TGF-β II receptors that antagonize TGF-β (truncated TGF-β II receptor, soluble TGF-β II receptor, etc.), MMP inhibitors such as batimastat, antibodies and antibody fragments that neutralize the above bioactive substances, substances that suppress the expression of the above bioactive substances, such as an siRNA, a ribozyme, an antisense nucleic acid (including RNA, DNA, PNA, and composites thereof), substances that have a dominant negative effect such as a dominant negative mutant, vectors expressing same, cell activation inhibitors such as a sodium channel inhibitor, cell-growth inhibitors such as alkylating agents (e.g. ifosfamide, nimustine hydrochloride, cyclophosphamide, dacarbazine, melphalan, ranimustine, etc.), antitumor antibiotics (e.g. idarubicin hydrochloride, epirubicin hydrochloride, daunorubicin hydrochloride, daunorubicin citrate, doxorubicin hydrochloride, pirarubicin hydrochloride, bleomycin hydrochloride, peplomycin sulfate, mitoxantrone hydrochloride, mitomycin C, etc.), antimetabolites (e.g. gemcitabine hydrochloride, enocitabine, cytarabine ocfosfate, a cytarabine formulation, tegafur/uracil, a tegafur/gimeracil/oteracil potassium mixture, doxifluridine, hydroxycarbamide, fluorouracil, methotrexate, and mercaptopurine, etc.), alkaloids such as etoposide, irinotecan hydrochloride, vinorelbine tartrate, docetaxel hydrate, paclitaxel, vincristine sulfate, vindesine sulfate, and vinblastine sulfate, and platinum complexes such as carboplatin, cisplatin, nedaplatin, etc., and apoptosis inducers such as compound 861 and gliotoxin. Furthermore, the ‘drug controlling the activity or growth of a CAF’ referred to in the present invention may be any drug that directly or indirectly promotes the physical, chemical, and/or physiological actions, etc. of a CAF directly or indirectly related to suppressing the onset and/or progression of a cancer.

Other examples of the ‘drug controlling the activity or growth of a CAF’ include drugs controlling the metabolism of an extracellular matrix, for example, collagen, and examples thereof include substances having an effect in suppressing the expression of a target molecule, such as an siRNA, a ribozyme, and an antisense nucleic acid (including RNA, DNA, PNA, or a composite thereof), which are targeted at an extracellular matrix constituent molecule produced by a CAF or targeted at one or more molecules involved in the production or secretion of the extracellular matrix constituent molecule, substances having a dominant negative effect such as a dominant negative mutant, and vectors expressing same.

Also within the scope of the invention are pharmaceutical formulations that include any of the aforementioned compounds in addition to a pharmaceutically acceptable carrier or diluent. Pharmaceutical formulations of the invention will include at least one therapeutic agent. Preferably, the therapeutic agent is an siRNA. It is envisioned that any siRNA molecule can be used within the scope of the invention. As previously described, siRNA include the sequences shown as SEQ. ID NO: 1, SEQ. ID NO: 2, SEQ. ID NO: 3, and SEQ. ID NO: 4.

In preferred formulations of the invention including siRNA, the siRNA is encapsulated by the liposome. In other embodiments, the siRNA can be outside of the liposome. In those embodiments, the siRNA can be complexed to the outside of the liposome.

A useful range of cationic lipid:siRNA (lipid nitrogen to siRNA phosphate ratio, “N:P”) is 0.2 to 5.0. A particularly preferred range of N:P is 1.5 to 2.5 for compositions and formulations of the description.

Preferred formulations of the invention include those comprising HEDC: S104:DOPE: Cholesterol:PEG-DMPE:DiVA-PEG-DiVA (20:20:30:25:5:2 molar ratio) and HEDC:S104:DOPE:Cholesterol:PEG-DMPE:DiVA-PEG-DiVA (20:20:30:25:5:2 molar ratio) wherein DiVA-PEG-DiVA is co-solubilized. DODC:DOPE:cholesterol:PEG-lipid:DiVA-PEG-DiVA (50:10:38:2:5 molar ratio) and DODC:DOPE:cholesterol:PEG-lipid:DiVA-PEG-DiVA formulations wherein the DiVA-PEG-DiVA is co-solubilized.

Other formulations of the invention include those comprising HEDODC:DOPE:cholesterol-PEG-lipid:DiVA-PEG-DiVA (50:10:38:2:5 molar ratio) and HEDODC:DOPE:cholesterol-PEG-lipid:DiVA-PEG-DiVA formulations wherein the DiVA-PEG-DiVA is co-solubilized.

Other preferred formulations of the invention include those comprising DC-6-14:DOPE:cholesterol: DiVA-PEG-DiVA (40:30:30:5, molar ratios) and DC-6-14:DOPE:cholesterol: DiVA-PEG-DiVA, wherein the DiVA-PEG-DiVA is co-solubilized.

Also within the scope of the invention are methods of delivering a therapeutic agent to a patient. These methods comprise providing a pharmaceutical formulation including any of the foregoing compositions and a pharmaceutically acceptable carrier or diluent; and administering the pharmaceutical formulation to the patient.

An siRNA is a double strand RNA having a sequence specific to a target molecule such as an mRNA, and suppresses the expression of a substance, for example, a protein, formed by the target molecule, by promoting the decomposition of the target molecule (RNA interference). Since Fire et al. published the principle (Nature, 391: 806-811, 1998), a wide range of research has been carried out into the optimization of siRNAs, and a person skilled in the art is familiar with such techniques. Furthermore, intensive research has been carried out into substances, other than siRNAs, that cause RNA interference or a gene expression inhibition reaction, and at present there are a large number of such substances.

For example, JP 2003-219893 A discloses a double strand polynucleotide formed from DNA and RNA that inhibits the expression of a target gene. This polynucleotide may be either a DNA/RNA hybrid in which one of the double strands is DNA and the other is RNA, or a DNA/RNA chimera in which a portion of the same strand is DNA and the other portion is RNA. Such a polynucleotide is preferably formed from 19 to 25 nucleotides, more preferably 19 to 23 nucleotides, and yet more preferably 19 to 21 nucleotides; in the case of a DNA/RNA hybrid it is preferable that the sense strand is DNA and the antisense strand is RNA, and in the case of a DNA/RNA chimera it is preferable that portion on the upstream side of the double strand polynucleotide is RNA. Such a polynucleotide may be prepared so as to have any sequence by a standard procedure of a known chemical synthetic method.

The target molecule is preferably a molecule that can completely suppress the production and/or secretion of an extracellular matrix constituent molecule, for example, and examples thereof include, without being limited thereto, HSP47. The gene sequence of HSP47 or a homologue thereof is disclosed as, for example, GenBank accession No. AB010273 (human), X60676 (mouse), and M69246 (rat, gp46).

Therefore, as the drug controlling the activity or growth of a CAF of the present invention, for example, an siRNA, a DNA/RNA hybrid, a chimeric polynucleotide, an antisense nucleic acid, etc, that are targeted at HSP47 are preferable.

The substance or body delivered by the carrier of the present invention may or may not be labeled. Labeling enables the success or failure of transport, increases and decreases in cancer cells or CAFs, etc. to be monitored, and is particularly useful at the testing/research level. A label may be selected from any label known to a person skilled in the art such as, for example, any radioisotope, magnetic material, a substance that binds to a labeling substance (e.g. an antibody), a fluorescent substance, a fluorophore, a chemiluminescent substance, an enzyme, etc.

In the present invention, ‘to a cancer cell’ or ‘to a cancer-associated fibroblast’ means that it is suitable to use cancer cells or cancer-associated fibroblasts as a target, and this includes it being possible to deliver a substance to a target cell, that is, a cancer cell or a cancer-associated fibroblast, more rapidly, efficiently, and/or in a larger quantity than to other cells (non-target cells), for example, a noncancer cell or a normal fibroblast. For example, the carrier of the present invention can deliver a substance to a cancer cell or a cancer-associated fibroblast at a rate and/or efficiency of at least 1.1 times, at least 1.2 times, at least 1.3 times, at least 1.5 times, at least 2 times, or even at least 3 times compared with other cells. The ‘efficiency’ referred to here means the proportion of cells to which a substance is delivered relative to all the cells of the evaluation target.

The present invention also relates to a composition that includes the targeting agent or carrier, and one or more types of the above-mentioned drugs controlling the activity or growth of a cancer cell and/or a CAF, the composition being for controlling the activity or growth of a cancer cell or for treating a cancer (anticancer composition), for controlling the activity or growth of a CAF (anti-CAF composition), or for treating a cancer in which CAF is involved, and use of the targeting agent or carrier in the production of these compositions.

In the present invention, the cancer is any malignant tumor, and examples thereof include fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, osteosarcoma and, furthermore, brain tumor, head and neck carcinoma, breast carcinoma, lung carcinoma, esophageal carcinoma, stomach carcinoma, duodenal carcinoma, appendiceal carcinoma, colon carcinoma, rectal carcinoma, hepatic carcinoma, pancreatic carcinoma, gallbladder carcinoma, bile duct carcinoma, anal carcinoma, kidney carcinoma, ureteral carcinoma, bladder carcinoma, prostate carcinoma, penile carcinoma, testicular carcinoma, uterine carcinoma, ovarian carcinoma, vulvar carcinoma, vaginal carcinoma, skin carcinoma, leukemia, and malignant lymphoma. The cancer may be or may not be accompanied by a CAF. In one embodiment of the present invention, the cancer is preferably a cancer other than hepatic carcinoma or pancreatic carcinoma. In another embodiment, the treatment of a cancer is preferably other than the prevention of hepatic carcinoma or pancreatic carcinoma.

Furthermore, the cancer in which CAF is involved in the present invention is not only a ‘CAF-accompanied cancer’ for which CAF is present in the interior or the periphery of the cancer, but also includes a cancer from which CAF is spatially separated but whose growth and activity are promoted by the above-mentioned bioactive substances released from CAF. Therefore, the cancer in which CAF is involved broadly means a malignant tumor, and includes any carcinoma, which is an epithelial malignant tumor, such as for example brain tumor, head and neck carcinoma, breast carcinoma, lung carcinoma, esophageal carcinoma, stomach carcinoma, duodenal carcinoma, appendiceal carcinoma, colon carcinoma, rectal carcinoma, hepatic carcinoma, pancreatic carcinoma, gallbladder carcinoma, bile duct carcinoma, anal carcinoma, kidney carcinoma, ureteral carcinoma, bladder carcinoma, prostate carcinoma, penile carcinoma, testicular carcinoma, uterine carcinoma, ovarian carcinoma, vulvar carcinoma, vaginal carcinoma, and skin carcinoma and, furthermore, any other malignant solid tumor, which is a nonepithelial malignant tumor, such as for example fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, and osteosarcoma. In the present invention, a cancer in which CAF is involved, selected from colorectal carcinoma, lung carcinoma, breast carcinoma, prostate carcinoma, stomach carcinoma, bile duct carcinoma, and a skin carcinoma such as basal cell carcinoma, can advantageously be treated due to a high degree of contribution of CAF to the growth. In one embodiment of the present invention, the cancer in which CAF is involved does not include hepatic carcinoma or pancreatic carcinoma. Furthermore, in another embodiment, the treatment of a cancer in which CAF is involved does not include the prevention of hepatic carcinoma or pancreatic carcinoma.

One embodiment of the anticancer composition of the present invention includes the targeting agent or the carrier, and a drug controlling the activity and growth of a cancer cell, and delivering this directly to a cancer cell allows an anticancer action to be exhibited. Another embodiment of the anticancer composition of the present invention includes the targeting agent or the carrier, and a drug controlling the activity or growth of a CAF, and delivering this to a CAF and controlling the activity or growth thereof allows an anticancer action to be exhibited indirectly. Yet another embodiment of the anticancer composition of the present invention includes the targeting agent or the carrier, and either one of a drug controlling the activity or growth of a cancer cell and a drug controlling the activity or growth of a CAF, or both thereof, and since the drug controlling the activity or growth of a cancer cell acts on a cancer cell, and the drug controlling the activity or growth of a CAF acts on a CAF, the anticancer action is doubled. In this embodiment, the drug controlling the activity or growth of a cancer cell and the drug controlling the activity or growth of a CAF may be identical to each other or different from each other.

In the composition of the present invention, as long as the targeting agent is present in a mode that allows a targeting function to be exhibited, the carrier may contain a substance to be carried within its interior, may be attached to the exterior of a substance to be carried, or may be mixed with a substance to be carried. Therefore, depending on the administration route, the manner in which the drug is released, etc., the composition may be covered with an appropriate material such as, for example, an enteric coating or a material that disintegrates over time, or may be incorporated into an appropriate drug release system.

In another aspect, the present disclosure relates to a pharmaceutical formulation comprising one or more physiologically acceptable surface active agents, pharmaceutical carriers, diluents, excipients, and suspension agents, or a combination thereof; and a formulation (e.g., the formulation that can include a compound, a retinoid and/or derivative thereof, a second lipid, a stabilizing agent, and/or a therapeutic agent) disclosed herein. Acceptable additional pharmaceutical carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, and the like may be provided in the pharmaceutical formulation. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium metasilicate aluminate, synthetic aluminum silicate, calcium carbonate, sodium acid carbonate, calcium hydrogen phosphate, calcium carboxymethyl cellulose, and the like may be used as excipients; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The pharmaceutical formulations described herein can be administered to a human patient per se, or in pharmaceutical formulations where they are mixed with other active ingredients, as in combination therapy, or suitable pharmaceutical carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may include, for example, parenteral delivery, including intramuscular, subcutaneous, intravenous, intramedullary injections, as well as intrathecal, direct intraventricular, intraperitoneal, intranasal, or intraocular injections. The formulation (e.g., the formulation that can include a compound, a retinoid and/or derivative thereof, a second lipid, a stabilizing agent, and/or a therapeutic agent) can also be administered in sustained or controlled release dosage forms, including depot injections, osmotic pumps, and the like, for prolonged and/or timed, pulsed administration at a predetermined rate. Additionally, the route of administration may be local or systemic.

The pharmaceutical formulations may be manufactured in a manner that is itself known, e.g., by means of conventional mixing, dissolving, granulating, dragee-making, levigating, emulsifying, encapsulating, entrapping or tableting processes.

Pharmaceutical formulations may be formulated in any conventional manner using one or more physiologically acceptable pharmaceutical carriers comprising excipients and auxiliaries which facilitate processing of the active compounds into preparations which can be used pharmaceutically. Proper formulation is dependent upon the route of administration chosen. Any of the well-known techniques, pharmaceutical carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, sucrose, glucose, dextrose, mannitol, lactose, lecithin, albumin, sodium glutamate, cysteine hydrochloride, and the like. In addition, if desired, the injectable pharmaceutical formulations may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations may be utilized.

Pharmaceutical formulations for parenteral administration, e.g., by bolus injection or continuous infusion, include aqueous solutions of the active formulation (e.g., the formulation that can include a compound, a retinoid and/or derivative thereof, a second lipid, a stabilizing agent, and/or a therapeutic agent) in water-soluble form. Additionally, suspensions of the active compounds may be prepared as appropriate oily injection suspensions. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition to the preparations described previously, the formulations may also be formulated as a depot preparation. Such long acting formulations may be administered by intramuscular injection. Thus, for example, the formulations (e.g., the formulation that can include a compound, a retinoid and/or derivative thereof, a second lipid, a stabilizing agent, and/or a therapeutic agent) may be formulated with suitable polymeric or hydrophobic materials (for example as an emulsion in an acceptable oil) or ion exchange resins, or as sparingly soluble derivatives, for example, as a sparingly soluble salt.

Some embodiments herein are directed to a method of delivering a therapeutic agent to a cell. For example, some embodiments are directed to a method of delivering a therapeutic agent such as siRNA into a cell. Suitable cells for use according to the methods described herein include prokaryotes, yeast, or higher eukaryotic cells, including plant and animal cells (e.g., mammalian cells). In some embodiments, the cells can be human fibrosarcoma cells (e.g., HT1080 cell line). In other embodiments, the cells can be cancer cells. Cell lines which are model systems for cancer may be used, including but not limited to breast cancer (MCF-7, MDA-MB-438 cell lines), U87 glioblastoma cell line, B16F0 cells (melanoma), HeLa cells (cervical cancer), A549 cells (lung cancer), and rat tumor cell lines GH3 and 9L. In these embodiments, the formulations described herein can be used to transfect a cell. These embodiments may include contacting the cell with a formulation described herein that includes a therapeutic agent, to thereby deliver a therapeutic agent to the cell.

The composition of the present invention may be administered via various routes including both oral and parenteral, and examples thereof include, but are not limited to, oral, intravenous, intramuscular, subcutaneous, local, rectal, intraarterial, intraportal, intraventricular, transmucosal, percutaneous, intranasal, intraperitoneal, intratumoral, intrapulmonary, and intrauterine routes, and it may be formulated into a dosage form suitable for each administration route. Such a dosage form and formulation method may be selected as appropriate from any known forms and methods (see e.g. Hyojun Yakuzaigaku (Standard Pharmaceutics), Ed. by Yoshiteru Watanabe et al., Nankodo, 2003).

Examples of dosage forms suitable for oral administration include, but are not limited to, powder, granules, tablet, capsule, liquid, suspension, emulsion, gel, and syrup, and examples of the dosage form suitable for parenteral administration include injections such as an injectable solution, an injectable suspension, an injectable emulsion, and an injection in a form that is prepared at the time of use. Formulations for parenteral administration may be a configuration such as an aqueous or nonaqueous isotonic aseptic solution or suspension.

The targeting agent, the carrier, or the composition of the present invention may be supplied in any configuration, but from the viewpoint of storage stability, it is preferably provided in a configuration that can be prepared at the time of use, for example in a configuration that allows a doctor and/or a pharmacist, a nurse, another paramedic, etc. to prepare it at the place of treatment or in the vicinity thereof. In this case, the targeting agent, the carrier, or the composition of the present invention is provided as one or more containers containing at least one essential constituent element therefor, and it is prepared prior to use, for example, within 24 hours prior to use, preferably within 3 hours prior to use, and more preferably immediately prior to use. When carrying out the preparation, a reagent, a solvent, preparation equipment, etc. that are normally available in a place of preparation may be used as appropriate.

The present invention therefore also relates to a preparation kit for the carrier or the composition, the kit including one or more containers containing singly or in combination a targeting agent, and/or a substance to be carried, and/or a carrier-constituting substance other than the targeting agent, and also to a constituent element necessary for the carrier or the composition provided in the form of such a kit. The kit of the present invention may contain, in addition to the above, instructions, an electronic recording medium such as a CD or DVD related to a process for preparing the targeting agent, the carrier, and the composition of the present invention, or an administration method, etc. Furthermore, the kit of the present invention may include all of the constituent elements for completing the targeting agent, the carrier, or the composition of the present invention, but need not always include all of the constituent elements. Therefore, the kit of the present invention need not include a reagent or a solvent that is normally available at a place of medical treatment, an experimental facility, etc. such as, for example, sterile water, physiological saline, or a glucose solution.

Also disclosed herein are methods for treating a condition characterized by abnormal fibrosis, which may include administering a therapeutically effective amount of a formulation described herein. Conditions characterized by abnormal fibrosis may include cancer and/or a fibrotic disease. Types of cancer that may be treated or ameliorated by a formulation described herein include, but are not limited to, lung cancer, pancreatic cancer, breast cancer, liver cancer, stomach cancer, and colon cancer. In an embodiment, the cancer that may be treated or ameliorated is pancreatic cancer. In another embodiment, the cancer that may be treated or ameliorated is lung cancer. Types of fibrotic disease that may be treated or ameliorated by a formulation described herein include, but are not limited to, hepatic fibrosis, hepatic cirrhosis, pancreatitis, pancreatic fibrosis, cystic fibrosis, vocal cord scarring, vocal cord mucosal fibrosis, laryngeal fibrosis, pulmonary fibrosis, idiopathic pulmonary fibrosis, cystic fibrosis, myelofibrosis, retroperitoneal fibrosis, and nephrogenic systemic fibrosis. In an embodiment, the condition that may be treated or ameliorated is hepatic fibrosis.

The formulations or pharmaceutical compositions described herein may be administered to the subject by any suitable means. Non-limiting examples of methods of administration include, among others, (a) administration via injection, subcutaneously, intraperitoneally, intravenously, intramuscularly, intradermally, intraorbitally, intracapsularly, intraspinally, intrasternally, or the like, including infusion pump delivery; (b) administration locally such as by injection directly in the renal or cardiac area, e.g., by depot implantation; as well as as deemed appropriate by those of skill in the art for bringing the active compound into contact with living tissue.

Pharmaceutical compositions suitable for administration include formulations (e.g., the formulation that can include a compound, a retinoid and/or derivative thereof, a second lipid, a stabilizing agent, and/or a therapeutic agent) where the active ingredients are contained in an amount effective to achieve its intended purpose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of composition effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be about 10 microgram/kg to about 100 mg/kg body weight, preferably about 100 microgram/kg to about 10 mg/kg body weight. Alternatively dosages may be based and calculated upon the surface area of the patient, as understood by those of skill in the art.

The exact formulation, route of administration and dosage for the pharmaceutical compositions can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). Typically, the dose range of the composition administered to the patient can be from about 0.5 to about 1000 mg/kg of the patient's body weight. The dosage may be a single one or a series of two or more given in the course of one or more days, as is needed by the patient. In instances where human dosages for compounds have been established for at least some condition, the dosages will be about the same, or dosages that are about 0.1% to about 500%, more preferably about 25% to about 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, a dose of about 0.1 mg to 2000 mg of each active ingredient, preferably about 1 mg to about 500 mg, e.g. 5 to 200 mg. In other embodiments, an intravenous, subcutaneous, or intramuscular dose of each active ingredient of about 0.01 mg to about 100 mg, preferably about 0.1 mg to about 60 mg, e.g. about 1 to about 40 mg is used. In cases of administration of a pharmaceutically acceptable salt, dosages may be calculated as the free base. In some embodiments, the formulation is administered 1 to 4 times per day. Alternatively the formulations may be administered by continuous intravenous infusion, preferably at a dose of each active ingredient up to about 1000 mg per day. As will be understood by those of skill in the art, in certain situations it may be necessary to administer the formulations disclosed herein in amounts that exceed, or even far exceed, the above-stated, preferred dosage range in order to effectively and aggressively treat particularly aggressive diseases or infections. In some embodiments, the formulations will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of formulation administered may be dependent on the subject being treated, on the subject's weight, the severity of the affliction, the manner of administration and the judgment of the prescribing physician.

Formulations disclosed herein (e.g., the formulation that can include a compound, a retinoid and/or derivative thereof, a second lipid, a stabilizing agent, and/or a therapeutic agent) can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound formulated in a compatible pharmaceutical carrier may also be prepared, placed in an appropriate container, and labeled for treatment of an indicated condition.

The present invention further relates to a method for controlling the activity or growth of a cancer cell or treating a cancer, and a method for controlling the activity or growth of a CAF or treating a cancer in which CAF is involved, the method including administering an effective amount of the composition to a subject that requires it. The effective amount referred to here is, in a method for treating a cancer, for example, an amount that suppresses the onset of a cancer, alleviates the symptoms, or delays or stops progression of the cancer, and is preferably an amount that prevents the onset of a cancer or cures a cancer. It is also preferably an amount that does not cause an adverse effect that exceeds the benefit from administration. Such an amount may be determined as appropriate by an in vitro test using cultured cells or by a test in a model animal such as a mouse, a rat, a dog, or a pig, and such test methods are well known to a person skilled in the art. Moreover, the dose of the targeting agent contained in the carrier and the dose of the drug used in the method of the present invention are known to a person skilled in the art, or may be determined as appropriate by the above-mentioned test, etc.

One embodiment of the cancer treatment method of the present invention involves administering the anticancer composition that includes a targeting agent or a carrier and a drug controlling the activity or growth of a cancer cell, and directly delivering the drug to the cancer cell, thus treating the cancer. Another embodiment of the cancer treatment method of the present invention involves administering the anticancer composition that includes a targeting agent or a carrier and a drug controlling the activity or growth of a CAF, and delivering the drug to a CAF so as to control the activity or growth thereof, thus indirectly treating the cancer. Yet another embodiment of the cancer treatment method of the present invention includes administering the anticancer composition that includes a targeting agent or a carrier and either one of a drug controlling the activity or growth of a cancer cell and a drug controlling the activity or growth of a CAF, or both thereof, and delivering the drug controlling the activity or growth of a cancer cell to a cancer cell and the drug controlling the activity or growth of a CAF to a CAF respectively, thus treating the cancer via two routes. In this embodiment, the drug controlling the activity or growth of a cancer cell and the drug controlling the activity or growth of a CAF may be identical to each other or different from each other.

In the method of the present invention, the specific dose of the composition administered may be determined while taking into consideration various conditions with respect to a subject that requires the treatment, such as for example the severity of the symptoms, general health condition of the subject, age, weight, gender of the subject, diet, the timing and frequency of administration, a medicine used in combination, reaction to the treatment, compliance with the treatment, etc.

As the administration route, there are various routes including both oral and parenteral administrations, and examples thereof include oral, intravenous, intramuscular, subcutaneous, local, rectal, intraarterial, intraportal, intraventricular, transmucosal, percutaneous, intranasal, intraperitoneal, intratumoral, intrapulmonary, and intrauterine routes.

The frequency of administration depends on the properties of the composition used and the above-mentioned condition of the subject, and may be a plurality of times per day (that is, 2, 3, 4, 5, or more times per day), once a day, every few days (that is, every 2, 3, 4, 5, 6, or 7 days, etc.), a few times per week (e.g. 2, 3, 4 times, etc. per week), every other week, or every few weeks (that is, every 2, 3, 4 weeks, etc.).

In the method of the present invention, the term ‘subject’ means any living individual, preferably an animal, more preferably a mammal, and yet more preferably a human individual. In the present invention, the subject may be healthy or affected by some disorder, and when treatment of a cancer is intended, it typically means a subject affected by a cancer or having a risk of being affected.

Furthermore, the term ‘treatment’ includes all types of medically acceptable preventive and/or therapeutic intervention for the purpose of the cure, temporary remission, or prevention of a disorder. For example, the term ‘treatment’ includes medically acceptable intervention for various purposes, including delaying or stopping the progression of a cancer, involution or disappearance of lesions, prevention of onset of a cancer, and prevention of recurrence.

The present invention also relates to a method for delivering a drug to a cancer cell and/or a CAF, utilizing the above carrier. This method includes, but is not limited to, for example, a step of supporting a substance to be carried on the carrier, and a step of administering or adding the carrier having the substance to be carried supported thereon to a living being or a medium, for example a culture medium, containing a cancer cell and/or a CAF. These steps may be achieved as appropriate in accordance with any known method or a method described in the present specification, etc. The above delivery method may be combined with another delivery method, for example, a delivery method targeted at an organ in which a cancer cell and/or a CAF is/are present. Moreover, the above method includes a mode carried out in vitro and a mode in which a cancer cell and/or a CAF inside the body is/are targeted.

### DEFINITIONS

As used herein, “alkyl” refers to a straight or branched fully saturated (no double or triple bonds) hydrocarbon group, for example, a group having the general formula —CₙH₂ₙ₊₁. The alkyl group may have 1 to 50 carbon atoms (whenever it appears herein, a numerical range such as “1 to 50” refers to each integer in the given range; e.g., “1 to 50 carbon atoms” means that the alkyl group may consist of 1 carbon atom, 2 carbon atoms, 3 carbon atoms, etc., up to and including 50 carbon atoms, although the present definition also covers the occurrence of the term “alkyl” where no numerical range is designated). The alkyl group may also be a medium size alkyl having 1 to 30 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl and the like.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution unless otherwise indicated.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution unless otherwise indicated.

As used herein, “halogen” refers to F, Cl, Br, and I.

As used herein, “mesylate” refers to —OSO₂CH₃.

As used herein, the term “pharmaceutical formulation” refers to a mixture of a composition disclosed herein with one or more other chemical components, such as diluents or additional pharmaceutical carriers. The pharmaceutical formulation facilitates administration of the composition to an organism. Multiple techniques of administering a pharmaceutical formulation exist in the art including, but not limited to injection and parenteral administration.

As used herein, the term “pharmaceutical carrier” refers to a chemical compound that facilitates the incorporation of a compound into cells or tissues. For example dimethyl sulfoxide (DMSO) is a commonly utilized carrier as it facilitates the uptake of many organic compounds into the cells or tissues of an organism

As used herein, the term “diluent” refers to chemical compounds diluted in water that will dissolve the formulation of interest (e.g., the formulation that can include a compound, a retinoid, a second lipid, a stabilizing agent, and/or a therapeutic agent) as well as stabilize the biologically active form of the formulation. Salts dissolved in buffered solutions are utilized as diluents in the art. One commonly, used buffered solution is phosphate buffered saline because it mimics the salt conditions of human blood. Since buffer salts can control the pH of a solution at low concentrations, a buffered diluent rarely modifies the biological activity of the formulation. As used herein, an “excipient” refers to an inert substance that is added to a formulation to provide, without limitation, bulk, consistency, stability, binding ability, lubrication, disintegrating ability, etc., to the composition. A “diluent” is a type of excipient.

As used herein, “therapeutic agent” refers to a compound that, upon administration to a mammal in a therapeutically effective amount, provides a therapeutic benefit to the mammal. A therapeutic agent may be referred to herein as a drug. Those skilled in the art will appreciate that the term “therapeutic agent” is not limited to drugs that have received regulatory approval. A “therapeutic agent” can be operatively associated with a compound as described herein, a retinoid, and/or a second lipid. For example, a second lipid as described herein can form a liposome, and the therapeutic agent can be operatively associated with the liposome, e.g., as described herein.

As used herein, “lipoplex formulations” refer to those formulations wherein the siRNA is outside of the liposome. In preferred lipoplex formulations, the siRNA is complexed to the outside of the liposome. Other preferred lipoplex formulations include those wherein the siRNA is accessible to any medium present outside of the liposome.

As used herein, “liposome formulations” refer to those formulations wherein the siRNA is encapsulated within the liposome. In preferred liposome formulations, the siRNA is inaccessible to any medium present outside of the liposome.

As used herein, the term “co-solubilized” refers to the addition of a component to the cationic lipid mixture before the empty vesicle is formed.

As used herein, the “decorated” refers to the addition of a component after vesicle formation.

As used herein, “DC-6-14” refers to the following cationic lipid compound:

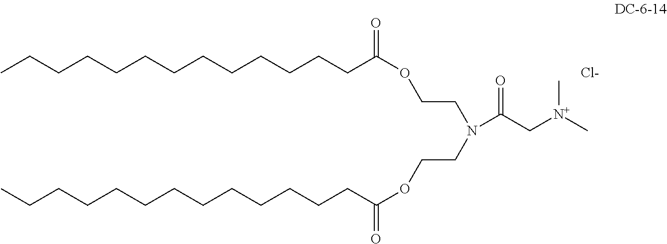 [SMILES: CCCCCCCCCCCCCC(=O)OCCN(CCOC(=O)CCCCCCCCCCCCC)C(=O)C[NH+](C)C ; [Cl-]]

As used herein, “DODC” refers to the following cationic lipid compound:

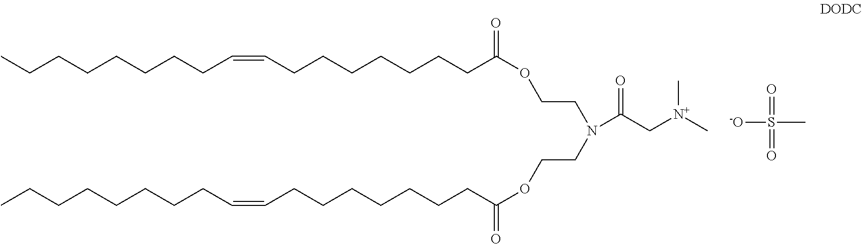

As used herein, “HEDODC” refers to the following cationic lipid compound:

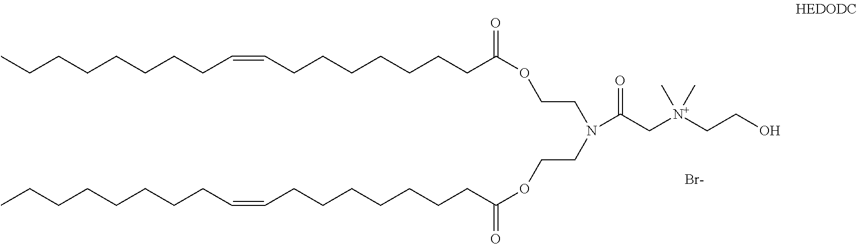 [SMILES: CCCCCCCC/C=C\CCCCCCCC(=O)OCCN(CCOC(=O)CCCCCCC/C=C\CCCCCCCC)C(=O)C[N+](C)(C)CCO ; [Br-]]

As used herein, a “retinoid” is a member of the class of compounds consisting of four isoprenoid units joined in a head-to-tail manner, see G. P. Moss, “Biochemical Nomenclature and Related Documents,” 2nd Ed. Portland Press, pp. 247-251 (1992). “Vitamin A” is the generic descriptor for retinoids exhibiting qualitatively the biological activity of retinol. As used herein, retinoid refers to natural and synthetic retinoids including first generation, second generation, and third generation retinoids. Examples of naturally occurring retinoids include, but are not limited to, (1) 11-cis-retinal, (2) all-trans retinol, (3) retinyl palmitate, (4) all-trans retinoic acid, and (5) 13-cis-retinoic acids. Furthermore, the term “retinoid” encompasses retinols, retinals, retinoic acids, rexinoids, demethylated and/or saturated retinoic acids, and derivatives thereof.

As used herein, “Vitamin D” is a generic descriptor for a group of vitamins having antirachitic activity. The vitamin D group includes: vitamin D₂ (calciferol), vitamin D₃ (irradiated 22-dihydroergosterol), vitamin D₄ (irradiated dehydrositosterol) and vitamin D₅ (irradiated dehydrositosterol).

As used herein, “Vitamin E” is a generic descriptor for a group of molecules with antioxidant activity. The vitamin E family includes α-tocopherol, β-tocopherol, γ-tocopherol and δ-tocopherol, with α-tocopherol being the most prevalent. (Brigelius-Flohe and Traber, The FASEB Journal. 1999; 13:1145-1155).

As used herein, “Vitamin K” is generic descriptor for an antihemorrahgic factor and includes vitamin K₁ (phytonodione), vitamin K₂ (menaquinone), vitamin K₃, vitamin K₄ and vitamin K₅. Vitamins K₁ and K₂ are natural, while K3-5 are synthetic.

As used herein, “retinoid-linker-lipid molecule” refers to a molecule that includes at least one retinoid moiety attached to at least one lipid moiety through at least one linker such as, for example, a PEG moiety.

As used herein, “retinoid-linker-retinoid molecule” refers to a molecule that includes at least one retinoid moiety attached to at least one other retinoid moiety (which may be the same or different) through at least one linker such as, for example, a PEG moiety.

As used herein, the terms “lipid” and “lipophilic” are used herein in their ordinary meanings as understood by those skilled in the art. Non-limiting examples of lipids and lipophilic groups include fatty acids, sterols, C₂-C₅₀ alkyl, C₂-C₅₀ heteroalkyl, C₂-C₅₀ alkenyl, C₂-C₅₀ heteroalkenyl, C₅-C₅₀ aryl, C₅-C₅₀ heteroaryl, C₂-C₅₀ alkynyl, C₂-C₅₀ heteroalkynyl, C₂-C₅₀ carboxyalkenyl, and C₂-C₅₀ carboxyheteroalkenyl. A fatty acid is a saturated or unsaturated long-chain monocarboxylic acid that contains, for example, 12 to 24 carbon atoms A lipid is characterized as being essentially water insoluble, having a solubility in water of less than about 0.01% (weight basis). As used herein, the terms “lipid moiety” and “lipophilic moiety” refers to a lipid or portion thereof that has become attached to another group. For example, a lipid group may become attached to another compound (e.g., a monomer) by a chemical reaction between a functional group (such as a carboxylic acid group) of the lipid and an appropriate functional group of a monomer.

As used herein, “siRNA” refers to small interfering RNA, also known in the art as short interfering RNA or silencing RNA. siRNA is a class of double stranded RNA molecules that have a variety of effects known in the art, the most notable being the interference with the expression of specific genes and protein expression.

As used herein, “encapsulated by the liposome” refers to a component being substantially or entirely within the liposome structure.

As used herein, “accessible to the aqueous medium” refers to a component being able to be in contact with the aqueous medium.

As used herein, “inaccessible to the aqueous medium” refers to a component not being able to be in contact with the aqueous medium.

As used herein, “complexed on the outer surface of the liposome” refers to refers to a component being operatively associated with the outer surface of the liposome.

As used herein, “localized on the outer surface of the liposome” refers to a component being at or near the outer surface of the liposome.

As used herein, “charge complexed” refers to an electrostatic association.

As used herein, the term “operatively associated” refers to an electronic interaction between a compound as described herein, a therapeutic agent, a retinoid, and/or a second lipid. Such interaction may take the form of a chemical bond, including, but not limited to, a covalent bond, a polar covalent bond, an ionic bond, an electrostatic association, a coordinate covalent bond, an aromatic bond, a hydrogen bond, a dipole, or a van der Waals interaction. Those of ordinary skill in the art understand that the relative strengths of such interactions may vary widely.

The term “liposome” is used herein in its ordinary meaning as understood by those skilled in the art, and refers to a lipid bilayer structure that contains lipids attached to polar, hydrophilic groups which form a substantially closed structure in aqueous media. In some embodiments, the liposome can be operatively associated with one or more compounds, such as a therapeutic agent and a retinoid or retinoid conjugate. A liposome may be comprised of a single lipid bilayer (i.e., unilamellar) or it may comprised of two or more concentric lipid bilayers (i.e., multilamellar). Additionally, a liposome can be approximately spherical or ellipsoidal in shape.

The term “facilitating drug delivery to a target cell” refers the enhanced ability of the present retinoid or fat soluble vitamin compounds to enhance delivery of a therapeutic molecule such as siRNA to a cell. While not intending to be bound by theory, the retinoid or fat-soluble vitamin compound interacts with a specific receptor (or activation/binding site) on a target cell with specificity that can be measured. For example, binding is generally consider specific when binding affinity (Kₐ) of 10⁶ M⁻¹ or greater, preferably 10⁷ M⁻¹ or greater, more preferably 10⁸M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660, 1949).

It is understood that, in any compound described herein having one or more stereocenters, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound having one or more double bond(s) generating geometrical isomers that can be defined as E or Z each double bond may independently be E or Z a mixture thereof. Likewise, all tautomeric forms are also intended to be included.

The present invention is explained more specifically by reference to Examples below, but the scope of the present invention is not limited by these Examples.

### EXAMPLES

### Example 1

### Separation of CAFs

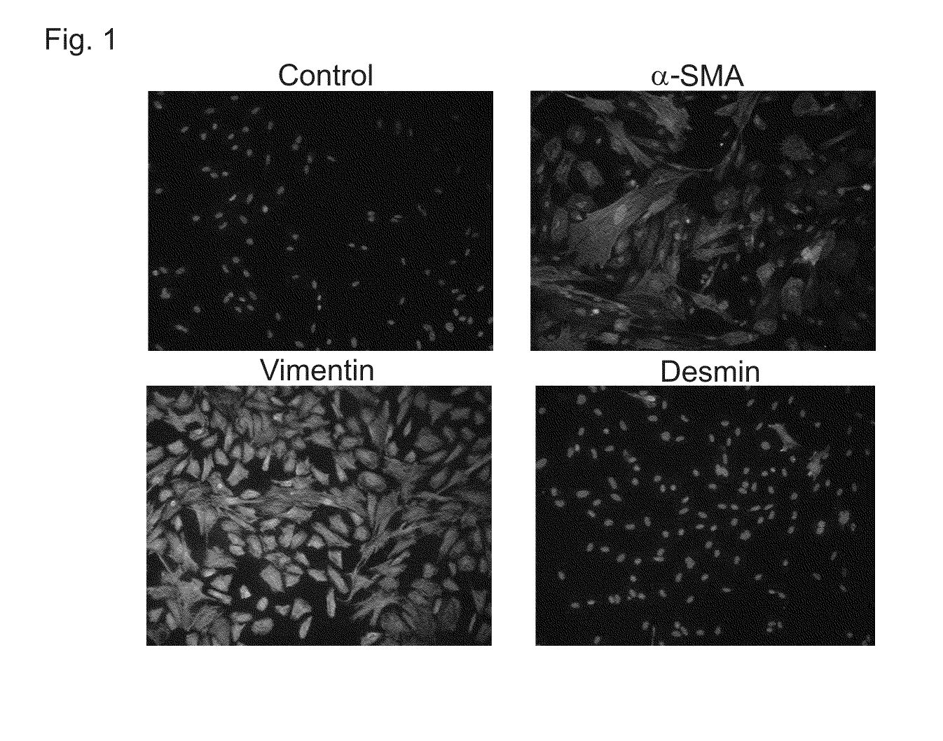

Cancer tissue or peripheral normal tissue (normal tissue separated from a site spaced from cancer tissue by at least 2 cm) removed from a colon cancer patient was finely cut into 1×1×1 mm, then centrifugally washed with PBS twice, and the pellets were cultured in a culture liquid (DMEM (Dulbecco's Modified Eagle Medium) containing collagenase type I (225 U/ml), hyaluronidase (125 U/ml), 10% FBS (fetal bovine serum), streptomycin/penicillin) for 24 hours. Subsequently, the supernatant was aspirated, and culturing was continued after changing the liquid culture for 10% FBS/DMEM. When the cultured cells were immunostained with an FITC labeled antibody with respect to α-SMA, which is a marker for CAFs, and vimentin, which is a marker for mesenchymal cells, α-SMA was detected only in cancer tissue-derived cells, and it was confirmed that these cells were CAFs (see FIG. 1). Vimentin was positive for cells derived from either tissue, and desmin, which is a marker for epithelial cells, was negative.

### Example 2

### CAF Tumor Growth Activity

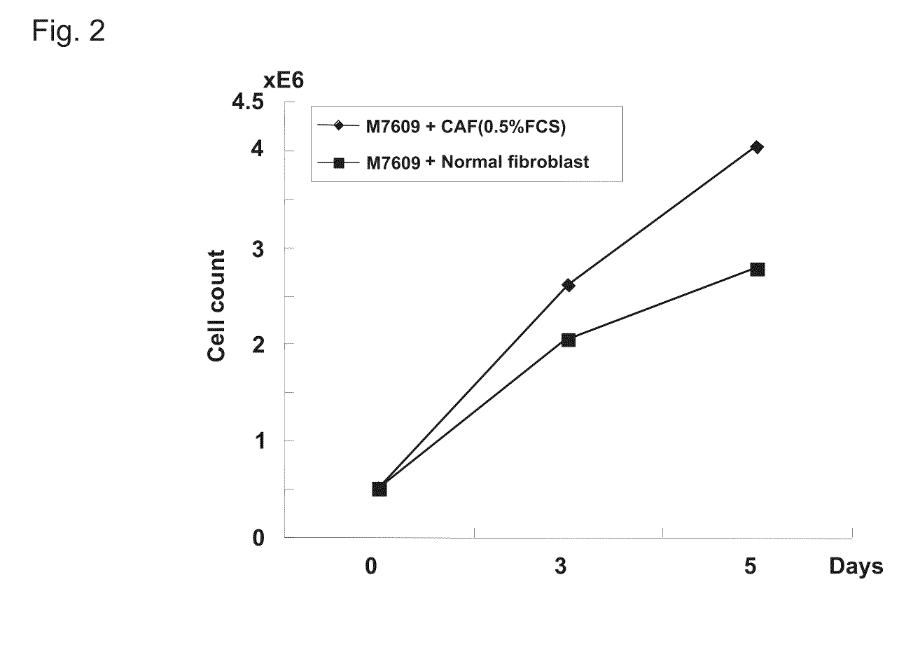

A 6-well plate was seeded with CAFs or normal fibroblasts obtained in Example 1 at a density of 1×10⁵ cells/well and cultured with 10% FBS/DMEM, the liquid culture was replaced with 0.5% FBS/DMEM in a confluent state on the third day, and the liquid culture was seeded with colon cancer cell line M7609 cells (2×10⁵ cells), and coculturing was carried out for 7 days. The number of M7609 cells was counted with a Coulter counter (Beckman) at 0 days and on the 3rd and 5th days. The results are given in FIG. 2. This shows that CAFs promote the growth of cancer cells.

### Example 3

### Preparation of siRNA

Three types of siRNA targeted at gp46 (GenBank Accession No. M69246), which is a rat homologue of human HSP47, and a random siRNA control were purchased from Hokkaido System Science Co., Ltd. Each siRNA consists of 27 bases overhanging on the 3′ side, and the sequences are as follows.

Sequence A:
(sense, SEQ. ID NO: 1)
5′-GUUCCACCAUAAGAUGGUAGACAACAG-3′
(antisense, SEQ. ID NO: 2)
5′-GUUGUCUACCAUCUUAUGGUGGAACAU-3′
Sequence B:
(sense, SEQ ID NO: 3)
5′-CCACAAGUUUUAUAUCCAAUCUAGCAG-3′
(antisense, SEQ. ID NO: 4)
5′-GCUAGAUUGGAUAUAAAACUUGUGGAU-3′
Sequence C:
(sense, SEQ ID NO: 5)
5′-CUAGAGCCAUUACAUUACAUUGACAAG-3′
(antisense, SEQ. ID NO: 6)
5′-UGUCAAUGUAAUGUAAUGGCUCUAGAU-3′
Random siRNA:
(sense, SEQ ID NO: 7)
5′-CGAUUCGCUAGACCGGCUUCAUUGCAG-3′
(antisense, SEQ. ID NO: 8)
5′-GCAAUGAAGCCGGUCUAGCGAAUCGAU-3′

Furthermore, siRNA labeled on the 5′ side with the fluorescent dye 6′-carboxyfluorescein (6-FAM) was also prepared.

### Example 4

### Preparation of siRNA-Containing VA-Bound Liposome

As a liposome, a cationic liposome containing DC-6-14, cholesterol, and DOPE at a molar ratio of 4:3:3 (Lipotrust, Hokkaido System Science Co., Ltd.) was used. 10 nmol of liposome and 20 nmol of all-trans retinol (hereinafter, referred to as ‘VA’) were mixed in DMSO using a 1.5 mL tube, then dissolved in chloroform, evaporated once, and then suspended in PBS. Subsequently, the siRNA (10 μg/mL) obtained in Example 3 and the liposome suspension were mixed at a ratio of 1:1 (w/w). Free VA and siRNA contained in the liposome suspension thus obtained were removed by a micropartition system (Sartorion VIVASPIN 5000MWCO PES), thus giving an siRNA-containing VA-bound liposome (VA-lip-siRNA). The amount of VA added and the amount of VA contained in the purified liposome were measured by HPLC, and when the proportion of VA bound to the liposome was examined, it was found that the majority of the VA (95.6±0.42%) was bound to the liposome. Furthermore, when the efficiency of uptake of siRNA into the liposome was measured by RiboGreen assay (Molecular Probes), it was 94.4±3.0%, which is high. Part of the VA was exposed on the surface of the liposome.

In the same manner as above, siRNA-containing liposome (lip-siRNA) and VA-bound liposome (VA-lip) were prepared.

### Example 5

### Uptake of VA-lip-siRNA

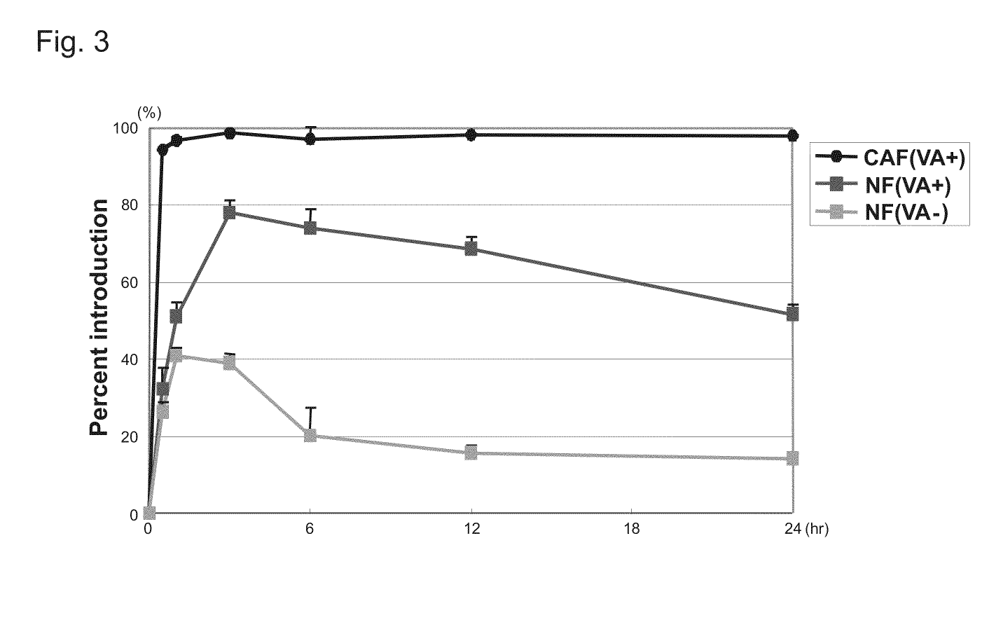

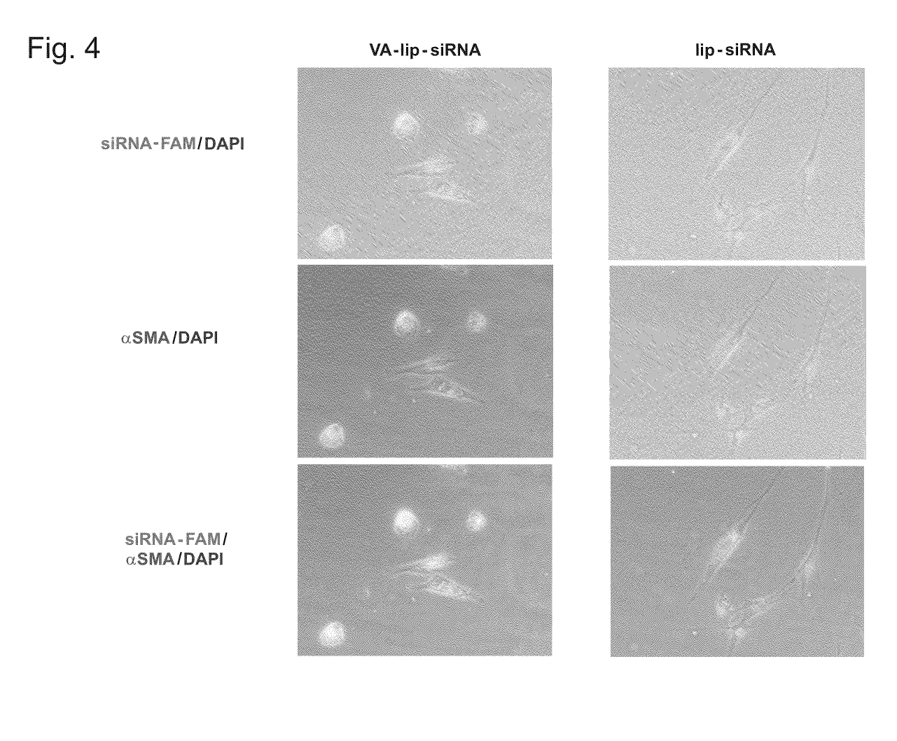

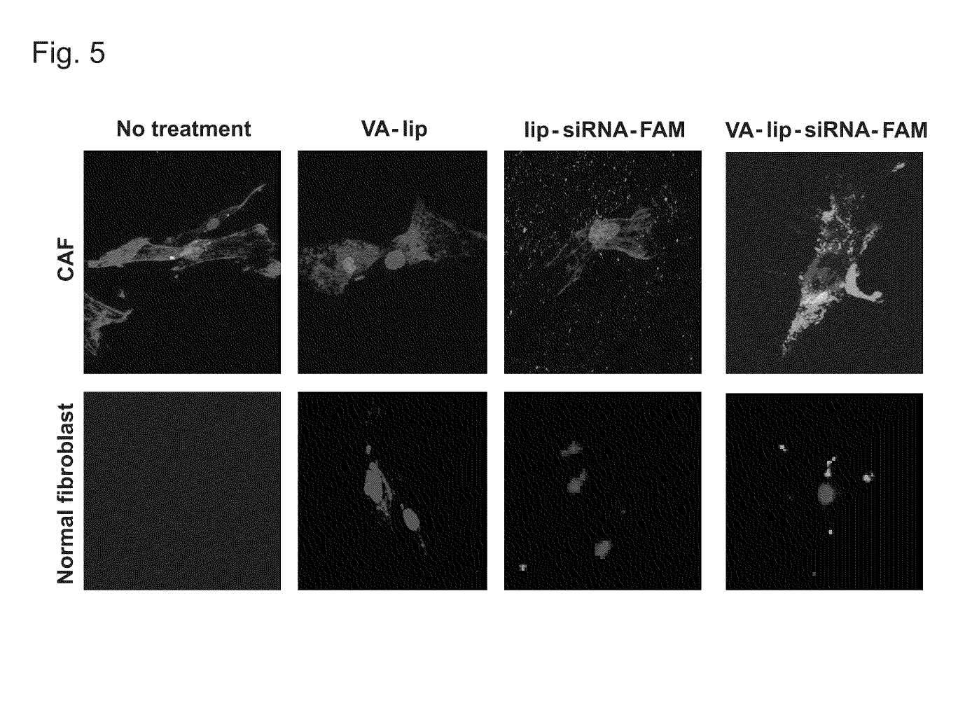

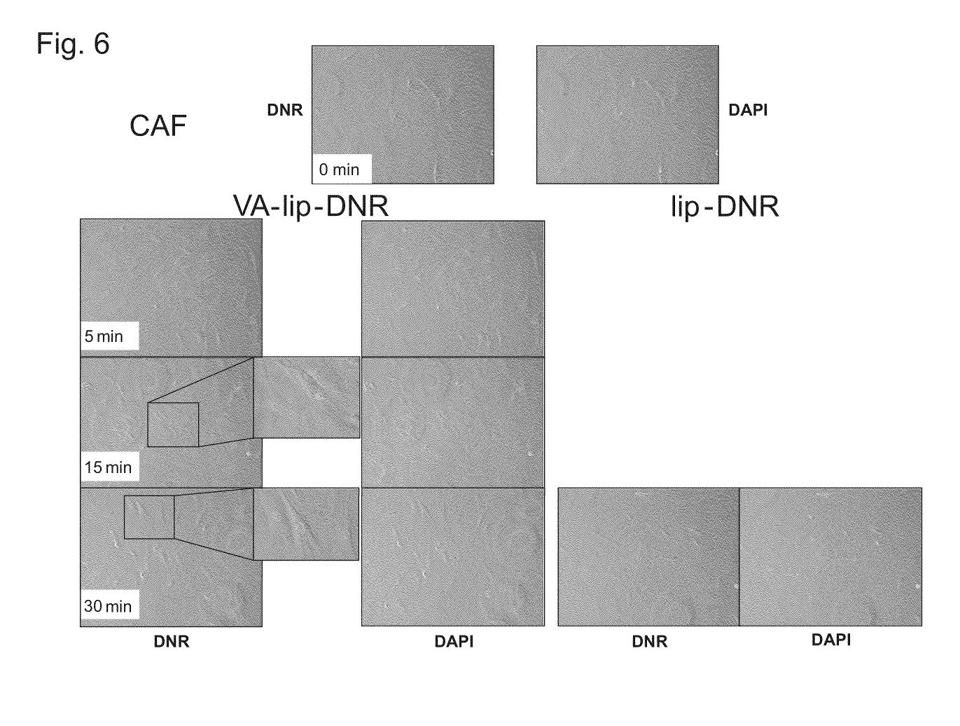

A 6-well plate was seeded with CAFs or normal fibroblasts at a density of 5×10⁵ cells/well, and culturing was carried out in 10% FBS/DMEM. After 2 days it was washed with serum-free medium in a subconfluent state, and the medium was replaced with serum-containing OPTI-MEM. Subsequently, the liposome suspension containing siRNA (final concentration 50 pmol/mL) obtained in Example 4 was added to the medium, and reacted at 37° C. for 24 hours. When the VA-bound liposome was added, the final concentration of VA was adjusted to 40 nmol/mL. Furthermore, as siRNA, 6-FAM labeled random siRNA was used. 0, 0.5, 1, 3, 6, 12, 18, and 24 hours after the reaction was started, the uptake of siRNA into each cell species was evaluated by flow cytometry (FIG. 3). After the reaction was complete, the cells were stained with DAPI (Molecular Probe) and Cy3-labeled anti α-SMA antibody, and the localization of siRNA was analyzed (FIG. 4 to 5).

As is clear from FIG. 3, it has been found that when the VA-containing carrier was used, the rate of transfer of siRNA into CAFs was at least 3 times the transfer rate into normal fibroblasts, the uptake by CAFs when 24 hours had elapsed was maintained at almost 100%, and the specificity and transfer efficiency were very high. Furthermore, FIG. 4 shows a representative field of vision used in evaluating the localization of siRNA, and according to this, when the VA-bound liposome (VA-lip-siRNA-FAM) was used, siRNA was incorporated into all of the CAFs in the field of vision, but when the liposome containing no VA (lip-siRNA-FAM) was used, siRNA was incorporated into only 1 CAF among 5 CAFs in the field of vision. Moreover, FIG. 5 shows that siRNA is not localized within the CAF cell for the liposome containing no VA (lip-siRNA-FAM), but most of the siRNA is localized within the cell for the VA-bound liposome (VA-lip-siRNA-FAM), and high efficiency transfer of siRNA into the CAF is VA dependent. From the above results, it is clear that the VA-containing carrier specifically and markedly promotes the uptake of a substance into CAF.

### Example 6

### Uptake of VA-lip-DNR

Uptake by CAFs was examined using VA-bound liposome containing daunorubicin (DNR) instead of siRNA.

Liposome encapsulated DNR (lip-DNR, DaunoXome®, hereinafter also called liposomal DNR) and VA were mixed in DMSO at a molar ratio of liposome:VA=1:2, then dissolved in chloroform, evaporated once, and then suspended in PBS. Free VA contained in the liposome suspension thus obtained was removed by a micropartition system (Sartorion VIVASPIN 5000MWCO PES), thus giving DNR-containing VA-bound liposome (VA-lip-DNR, hereinafter also called VA-bound liposomal DNR). The amount of VA added and the amount of VA contained in the purified liposome were measured by HPLC, and when the proportion of VA bound to the liposome was examined, it was found that the majority of the VA (98%) was bound to the liposome. Part of the VA was exposed on the surface of the liposome. In DaunoXome®, daunorubicin citrate is encapsulated in a liposome formed from distearoyl phosphatidylcholine (DSPC) and cholesterol (Chol), and the molar ratio of DSPC:Chol:daunorubicin citrate is 10:5:1.

A chamber slide was seeded with, the CAFs obtained in Example 1, normal fibroblasts, or commercial fibroblasts (skin fibroblast, Cells System, product No. CS-2F0-101) respectively at a density of 2×10⁴ cells/chamber, cultured with 10% FBS/DMEM overnight, then washed with serum-free medium once in a subconfluent state, and the medium was replaced with serum-containing OPTI-MEM. Subsequently, a liposome suspension containing lip-DNR or the VA-lip-DNR obtained above at 5 μg/mL as a DaunoXome® concentration was added to medium and reacted at 37° C. Furthermore, nuclei were stained with DAPI. The localization of DNR, which exhibited a red color, was examined under a fluorescence microscope before the reaction started (0 min), and 5 minutes, 15 minutes, and 30 minutes after the reaction started. The results are given in FIGS. 6 to 9.

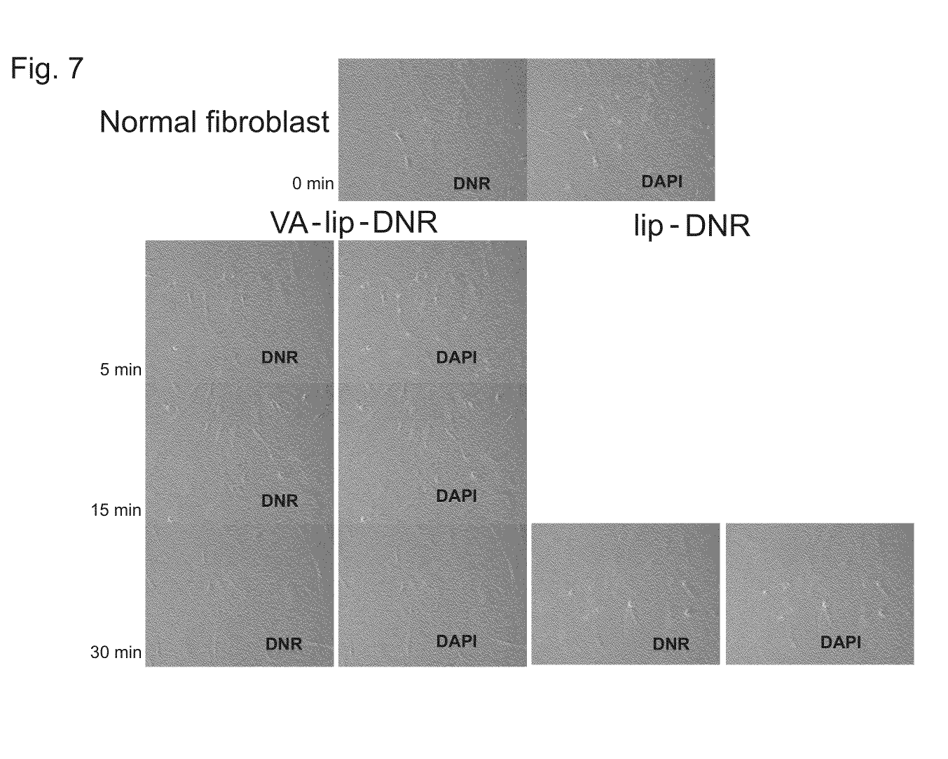

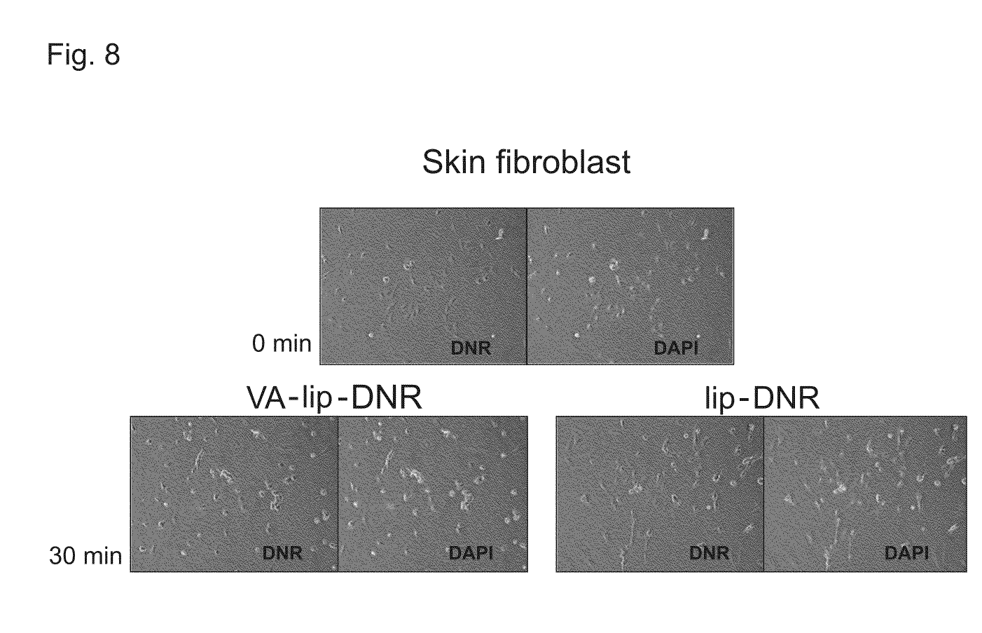

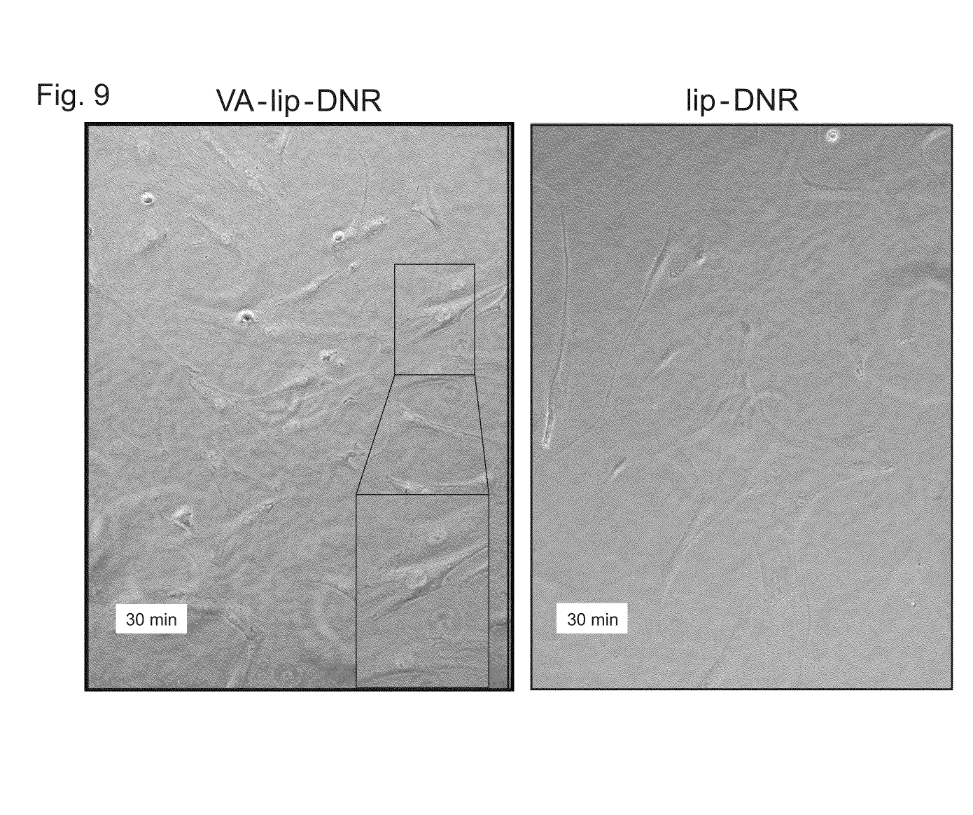

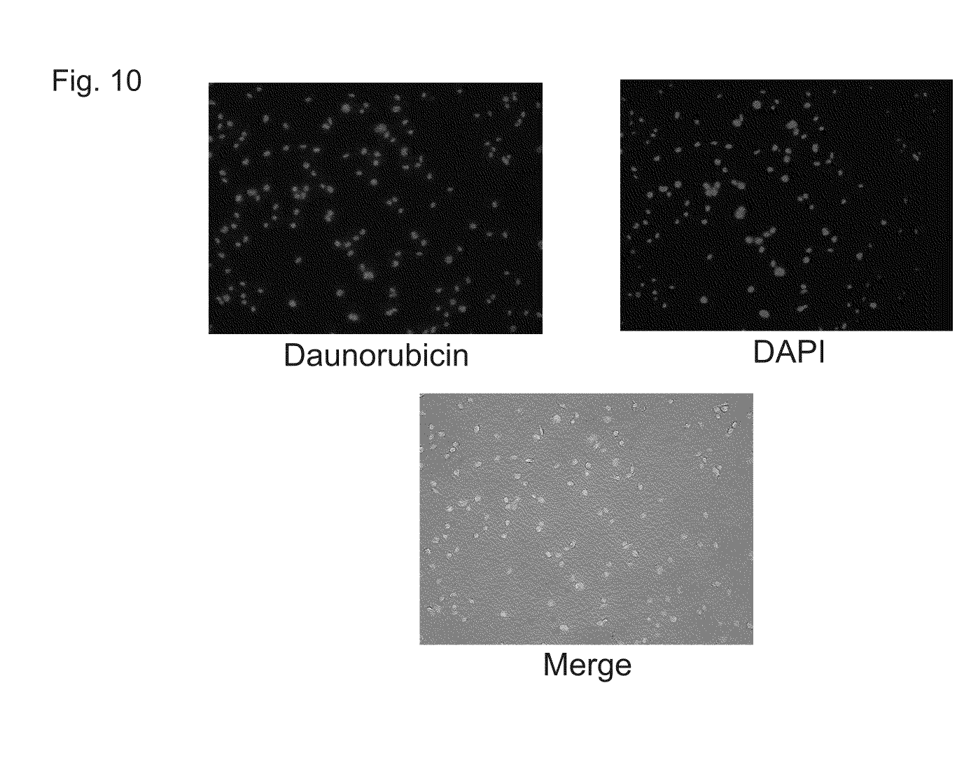

In CAFs to which VA-lip-DNR was added, a red color was already observed within the cell at 15 minutes after the addition, but in a group to which lip-DNR was added localization of DNR into the cells was not observed (FIGS. 6 and 9). Furthermore, in normal fibroblasts (FIG. 7) and skin fibroblasts (FIG. 8), localization of DNR was not observed either in the group to which VA-lip-DNR was added or in the group to which lip-DNR was added. These results show that the VA-bound carrier causes CAF-specific drug delivery.

### Example 7

### Targeting of the VA Derivative Retinoic Acid (RA) at Cancer Cells and CAFs

### (1) Cultured Cells

CAF cells were established by cloning from a clinical sample of a human cancer patient. HT-1080 human fibrosarcoma cells (fibrosarcoma), and HepG2 human hepatic cancer-derived cells were purchased from American Type Culture Collection. All cells were cultured with a DMEM medium (Sigma Aldrich) to which 10% fetal bovine serum (FBS) was added. They were trypsinized, a 4-well culture slide (BD Falcon #354114) was then seeded therewith at 2×10⁵ cells/mL, and cultured overnight under conditions of 37° C. and 5% CO₂.

### (2) Preparation of VA-Containing Liposomal Formulation

As a model drug, DaunoXome® (Gilead Sciences, Inc.), which is a liposome encapsulated daunorubicin formulation, was used. DaunoXome® contains the drug daunorubicin at a concentration of 2 mg/mL. 990 μL of 10% FBS-containing DMEM was added to 10 pit of DaunoXome®, thus giving a 20 μg/mL solution. This was mixed with 7.14 μL of all-trans retinol (VA) and all-trans retinoic acid (Retinoic acid, RA) dissolved in dimethylsulfoxide (DMSO) to give 100 mM, thus giving a VA-containing liposomal formulation (VA+) and an RA-containing liposomal formulation (retinoic acid+) respectively. At least part of the VA and the RA was exposed on the surface of the liposome. In addition to these liposomal formulations, as a control group a formulation (VA−), which was a DaunoXome® solution containing no VA or RA, was prepared.

### (3) Administration of VA and RA Liposomal Formulations

The medium was removed from the culture slide, and 750 μL of fresh 10% FBS-containing DMEM was added thereto. Except for the culture slide that had no treatment (No treatment), 250 μL of formulation (VA−), which was the DaunoXome® solution containing no VA or RA, the VA-containing liposomal formulation (VA+), and the RA-containing liposomal formulation (retinoic acid+) respectively were added and incubated under conditions of 37° C. and 5% CO₂ for 15 minutes. The medium was removed from each of the culture slides, they were washed with 1 mL of PBS twice, subsequently 1 mL of a 4% paraformaldehyde solution (Wako Pure Chemical Industries, Ltd.) was added thereto, and the cells were fixed at room temperature for 5 minutes. The fixing solution was then removed, and the cells were washed with PBS three times. The slide glass was taken out from each culture slide, Prolong Gold (Invitrogen) was added dropwise, and the slide glass was sealed with a cover glass.

### (4) Microscopic Examination

The slide glass was examined using a fluorescence microscope (Keyence BZ8000). It is known that daunorubicin is incorporated into the nucleus of a cell and emits red fluorescence under green excitation light (FIG. 10 upper left). Furthermore, the Prolong Gold used when sealing contains the fluorescent dye DAPI. DAPI binds to the nucleus of a cell and exhibits blue fluorescence under UV excitation light (FIG. 10 upper right). Therefore, in microscopic examination, when there is uptake of a liposomal formulation, both red and blue fluorescence is observed, and as a result a purple color is exhibited when superimposing the two images (FIG. 10 lower). On the other hand, when a liposomal formulation is not incorporated into a cell, only blue fluorescence due to DAPI is detected.

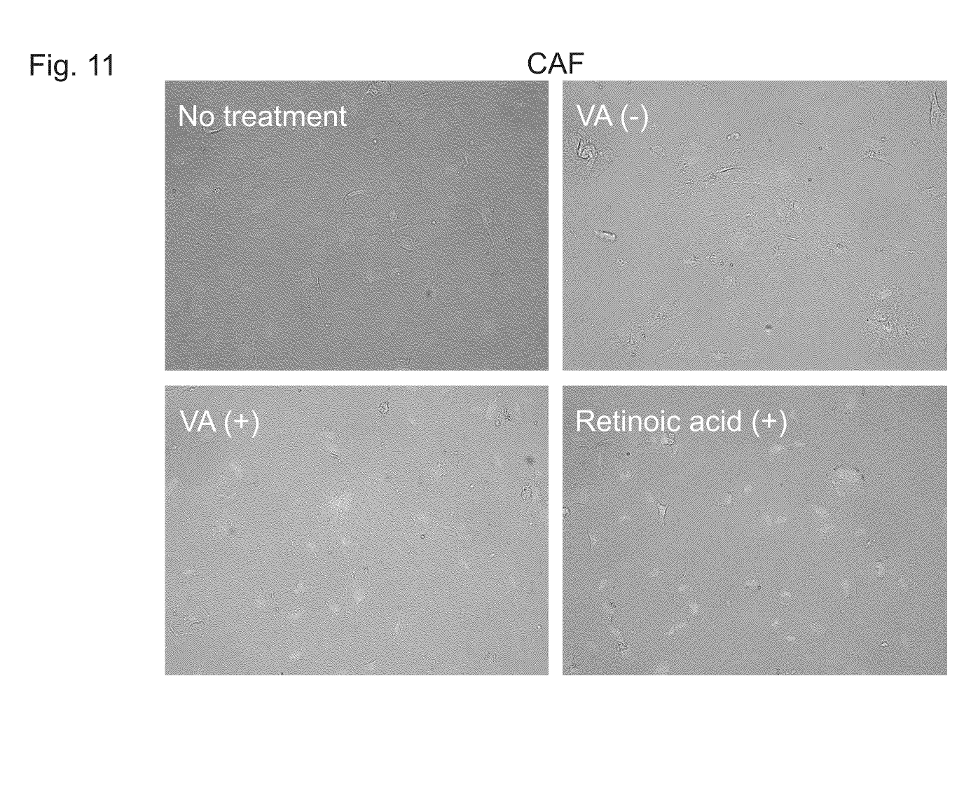

The slide glass was examined by a phase contrast microscope and the fluorescence microscope. An image of the slide glass taken by the phase contrast microscope under bright field, an image taken by the fluorescence microscope under green excitation light, and an image taken under UV excitation light were electronically merged (merge). Merged images are shown in FIGS. 11 to 13, and the observation results are shown in Tables 2 to 4. In the tables, + denotes that fluorescence was observed, and − denotes that fluorescence was not observed.

TABLE 2
Uptake of liposomal formulation in CAFs (FIG. 11)

| | DAPI (blue | Daunorubicin |
|---|---|---|
| | fluorescence) | (red fluorescence) |
| No treatment (No treatment) | + | − |
| DaunoXome ® (VA−) | + | − |
| DaunoXome ® + retinol (VA+) | + | + |
| DaunoXome ® + retinoic acid | + | + |
| (Retinoic acid+) | | |

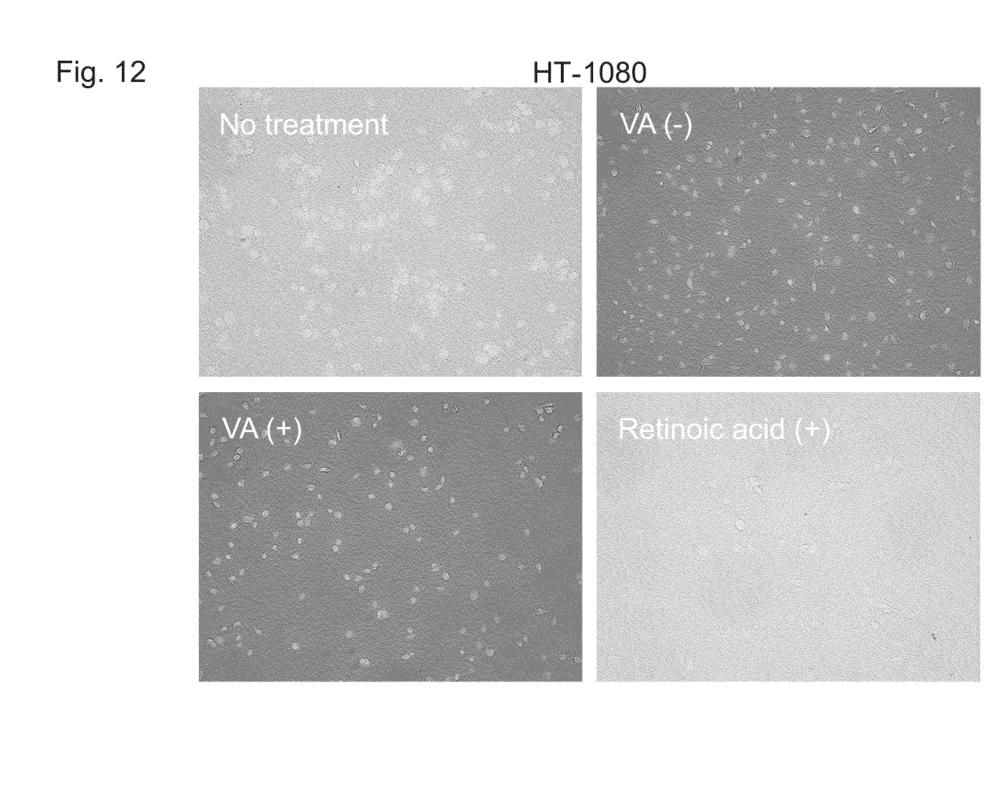

TABLE 3
Uptake of liposomal formulation in HT-1080 (FIG. 12)

| | DAPI (blue | Daunorubicin |
|---|---|---|
| | fluorescence) | (red fluorescence) |
| No treatment (No treatment) | + | − |
| DaunoXome ® (VA−) | + | − |
| DaunoXome ® + retinol (VA+) | + | + |
| DaunoXome ® + retinoic acid | + | + |
| (Retinoic acid+) | | |

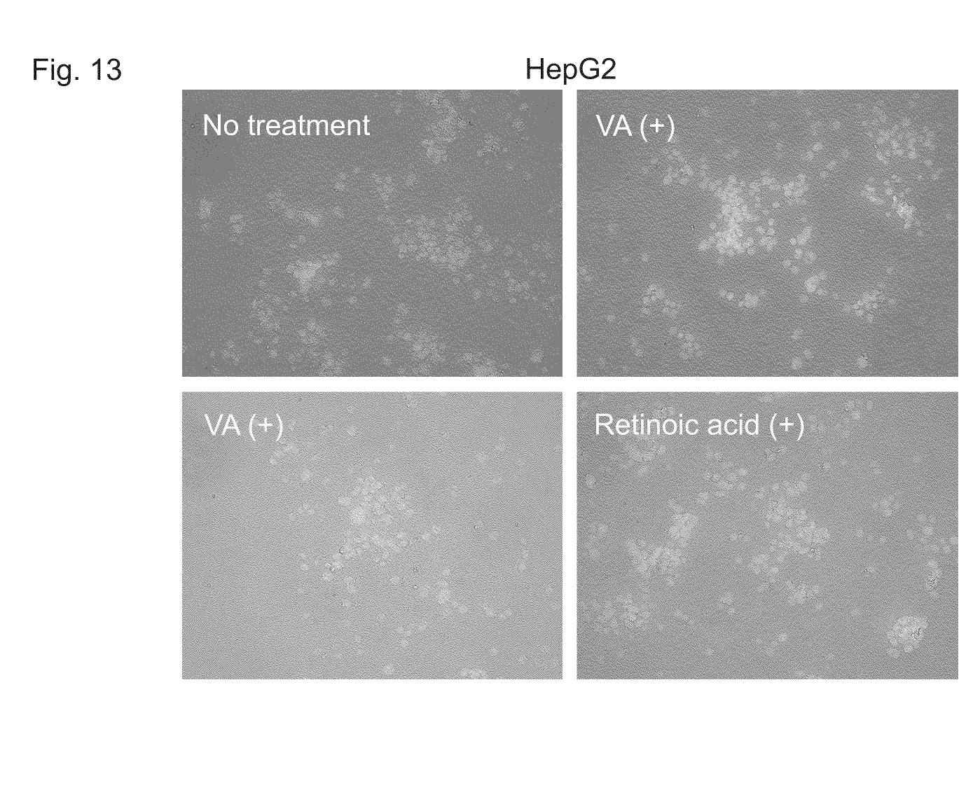

TABLE 4
Uptake of liposomal formulation in HepG2 (FIG. 13)

| | DAPI (blue | Daunorubicin |
|---|---|---|
| | fluorescence) | (red fluorescence) |
| No treatment (No treatment) | + | − |
| DaunoXome ® (VA−) | + | − |
| DaunoXome ® + retinol (VA+) | + | + |
| DaunoXome ® + retinoic acid | + | + |
| (Retinoic acid+) | | |

As is clear from these results, the presence of cell nuclei was confirmed for all the slide glasses due to the blue fluorescence of DAPI. The red fluorescence of daunorubicin showed that in the slide glasses employing VA and RA, localization of daunorubicin in cell nuclei was observed even after an incubation of as little as 15 minutes. In contrast thereto, in the slide glass employing no VA or RA, there was no localization of daunorubicin in the cell nucleus. This suggests that a retinoid can be used as a targeting agent to a CAF or a cancer cell.

### Example 8

### CAF-Specific Growth Inhibition by VA-Bound Liposome Encapsulated Drug

The CAF growth inhibitory activity of VA-bound liposome containing siRNA toward gp46 or DNR was examined.

### (1) Growth Inhibition by VA-lip-siRNA

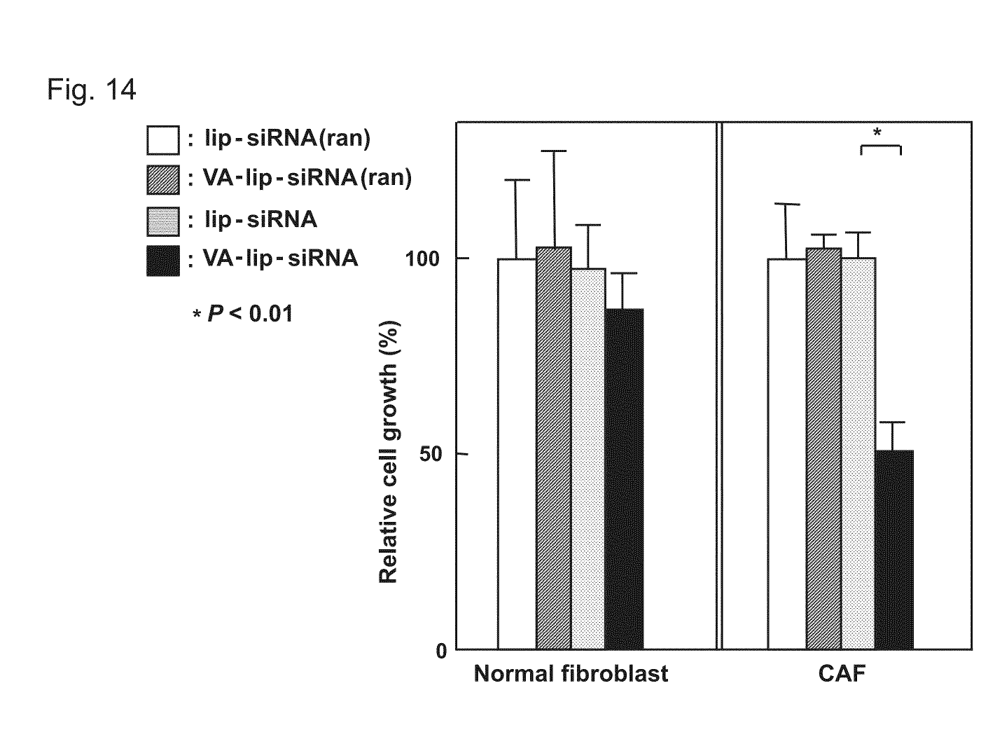

As the siRNA, sequence A described in Example 3 was used. A 24-well dish was seeded with CAFs and normal fibroblasts respectively at 1×10⁴ cells and cultured with 10% FBS/DMEM for 24 hours, VA-lip-siRNA was added at a final concentration of 50 pmol/mL, incubation was carried out for 1 hour, and subsequently the cells were washed. The viable cell count was measured by the WST-1 method after culturing with 10% FBS/DMEM for 48 hours. As a control, lip-siRNA- and random siRNA-containing VA-bound and nonbound liposomes (VA-lip-siRNA (ran) and lip-siRNA (ran)) were used, and evaluation of significant difference was carried out by the U test. The results are given in FIG. 14. From this figure, it can be seen that in CAFs to which VA-lip-siRNA was added the viable cell count greatly decreased to less than 50% of that prior to the treatment, but in the other treatment groups there was hardly any change in the viable cell count.

### (2) Growth Inhibition by VA-lip-DNR

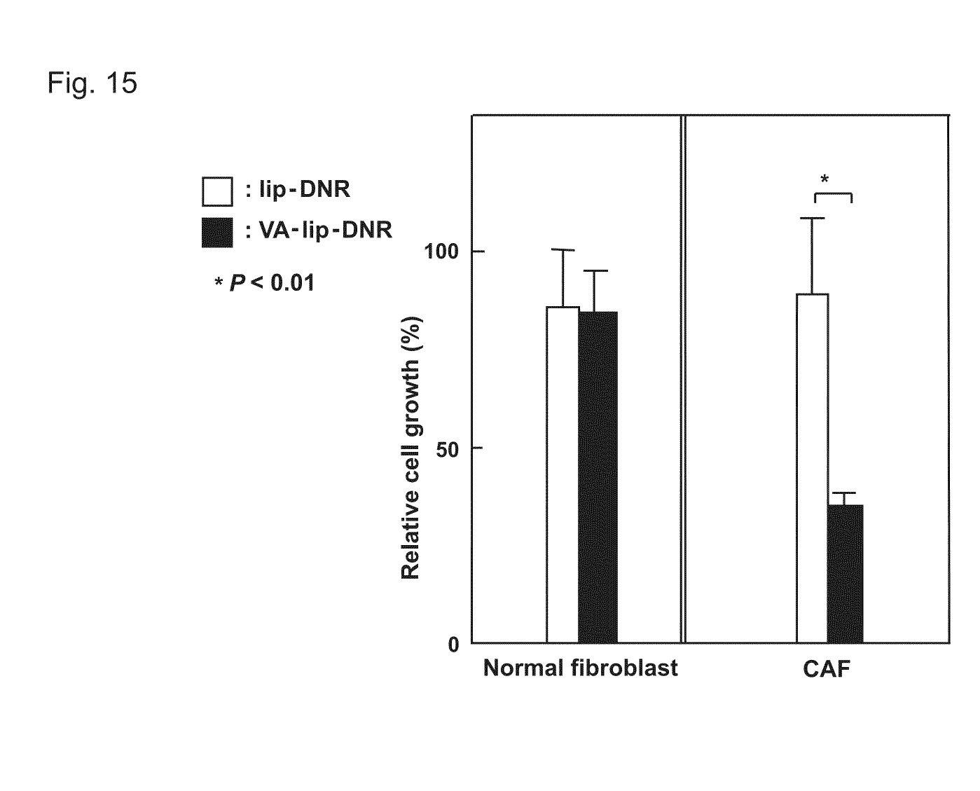

A 96-well dish was seeded with CAFs or normal fibroblasts respectively at 2×10³ cells, and cultured with 10% FBS/DMEM for 24 hours, subsequently the VA-lip-DNR obtained in Example 6 or lip-DNR was added at a final DaunoXome® concentration of 5 μg/mL and after exposing for 15 minutes, the cells were washed. Culturing was carried out with 10% FBS/DMEM for 24 hours, and the viable cell count was measured by the WST-1 method. Evaluation of significant difference was carried out by the U test. The results are shown in FIG. 15. From this figure, it can be seen that in CAFs to which VA-lip-DNR was added the viable cell count greatly decreased to about 40% of that prior to the treatment, but in the CAFs to which lip-DNR was added or normal fibroblasts there was hardly any change in the viable cell count.

The above results suggest that a drug supported on a VA-bound carrier exhibits a CAF-specific growth inhibitory activity.

### Example 9

### Examination of Efficiency of Incorporating VA-lip-DNR into Cancer Cells

Chamber slides (Falcon) were seeded with human fibrosarcoma-derived cell lines HT-1080, HS913T, and Sw684, human breast cancer-derived cell line MCF7, human osteosarcoma-derived cell line Saos2 (all purchased from ATCC), and human hepatic cancer-derived cell line Huh7 (purchased from JCRB Cell Bank) at a cell density of 1×10⁴ cells/well, cultured overnight, and washed with 10% FBS-containing DMEM. Subsequently, 5 μg/ml (8.85 μM as daunorubicin, 89.25 μM as liposome) of lip-DNR (DaunoXome®) or 5 μg/mL of the VA-lip-DNR (containing 178.5 μM of retinol) obtained in Example 6 was added thereto, the cells were washed 15 minutes and/or 30 minutes after the addition, and fixed by 4% formaldehyde. After washing with PBS, sealing was carried out with Prolong Gold (Invitrogen), and localization of DNR was examined by a fluorescence microscope.

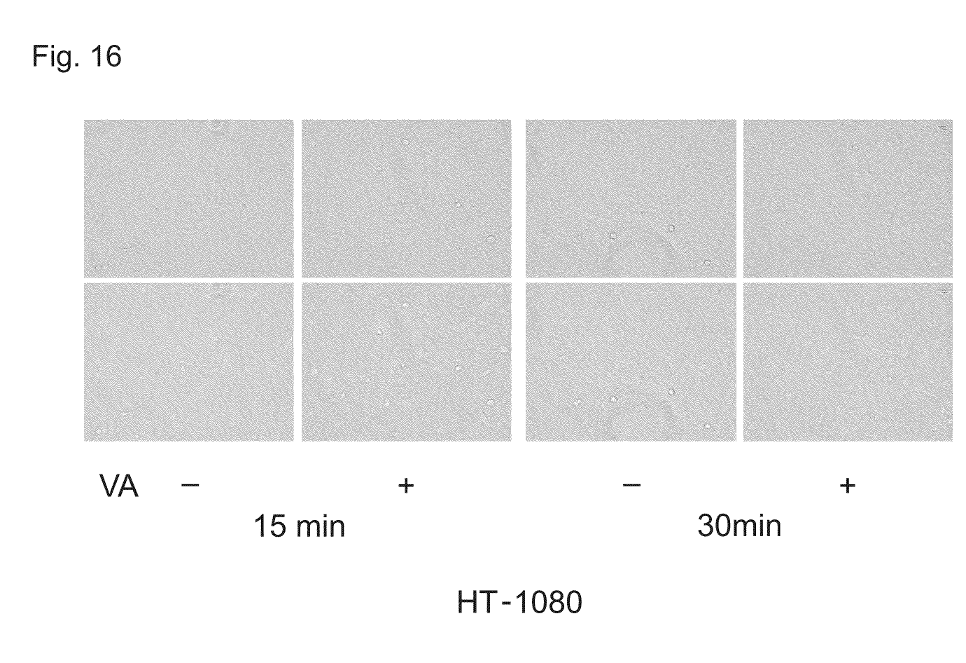

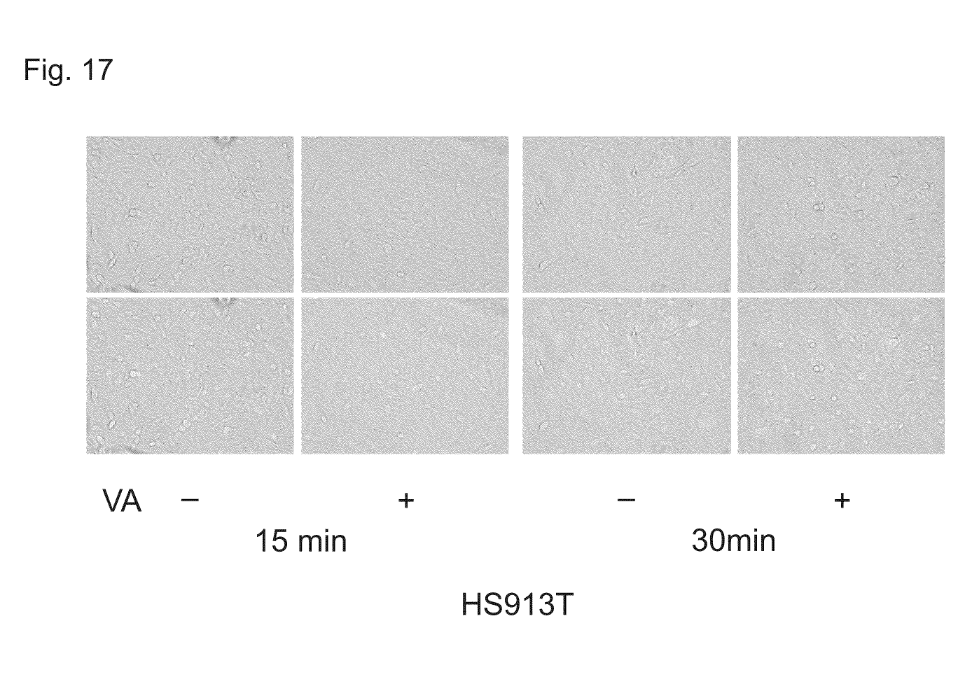

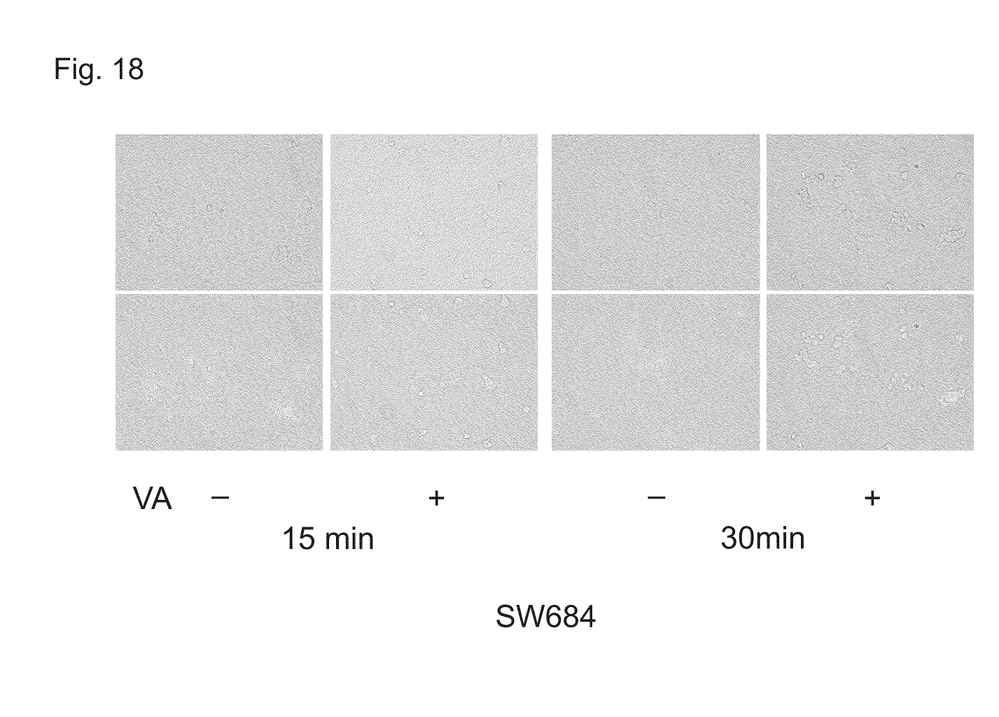

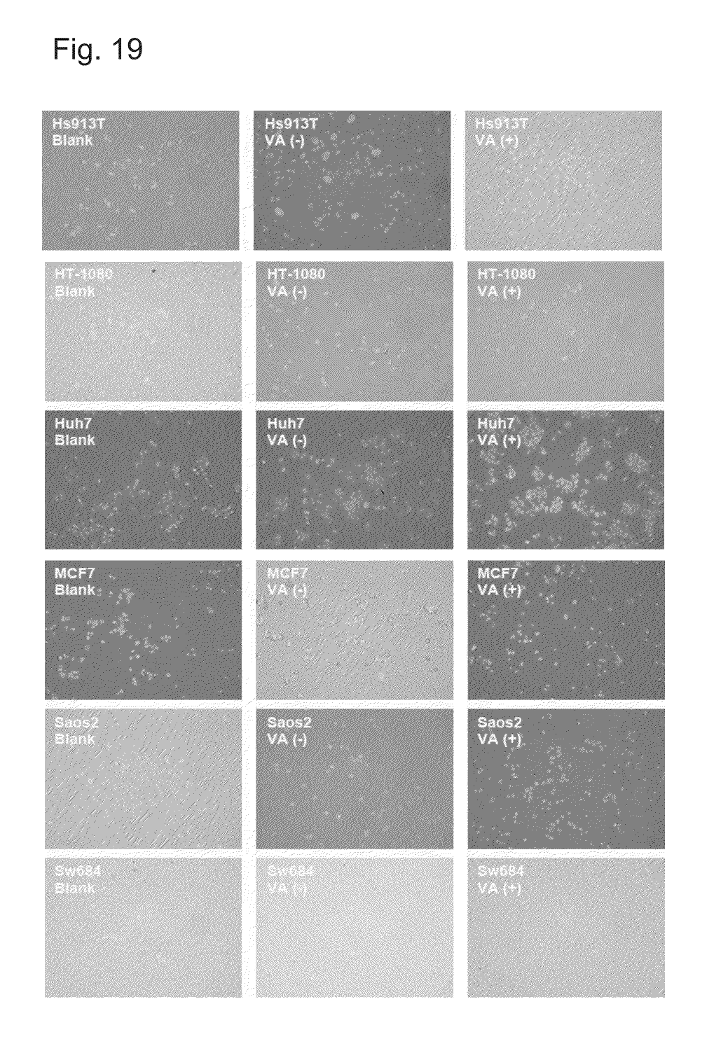

From the results shown in FIGS. 16 to 18, in all of the cells, in the VA-lip-DNR addition group, DNR, which exhibits a red color under a fluorescence microscope, was localized in the interior of the majority of cells only 15 minutes after the addition, whereas hardly any lip-DNR was incorporated even after 30 minutes had elapsed. This suggests that binding of VA greatly promotes the uptake of liposomal DNR into a cell. Furthermore, from the result shown in FIG. 19, it becomes clear that the above-mentioned promoting effect is observed in various cancer cells, including sarcoma and carcinoma cells.

### Example 10

### Examination of Antitumor Effect of VA-Bound Liposomal Daunorubicin

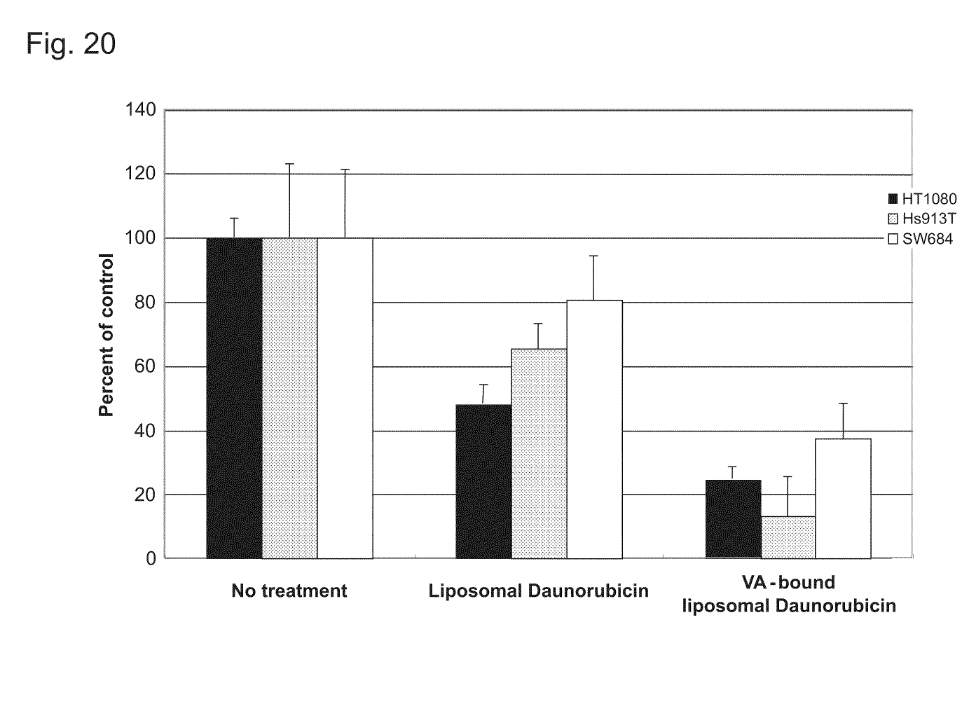

A 96-well plate was seeded with human fibrosarcoma-derived cell lines HT-1080, HS913T, and Sw684 at a cell density of 2×10³ cells/well and cultured overnight, subsequently 5 μg/mL of lip-DNR or 5 μg/mL of the VA-lip-DNR used in Example 6 was added, and culturing was carried out for 15 minutes. Following this, the cells were washed so as to remove drug that was outside the cells, and then cultured with 10% FBS-containing DMEM for 22 hours. 2 hours after WST-1 Cell Proliferation Assay Kit (Cayman Chemical) was added thereto, the absorbance was measured, and the proportion relative to the number of cells when the treatment was not carried out was calculated. From the result shown in FIG. 20, it can be seen that the binding of VA remarkably increases the antitumor activity of liposomal DNR.

### Example 11

### In Vivo CAF-Specific Delivery

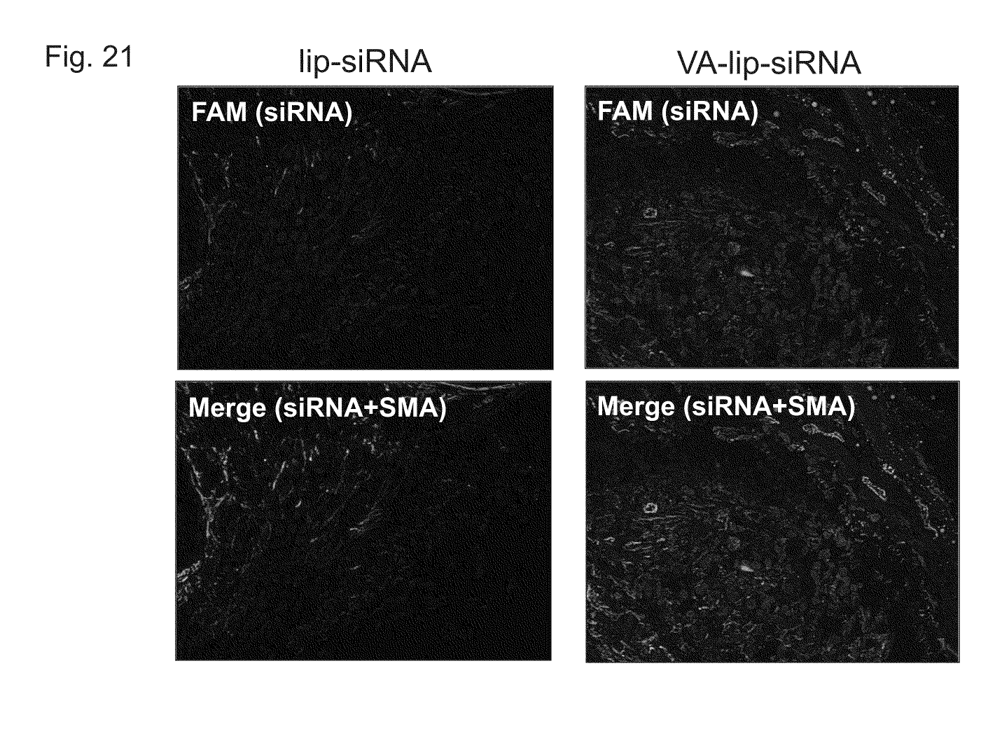

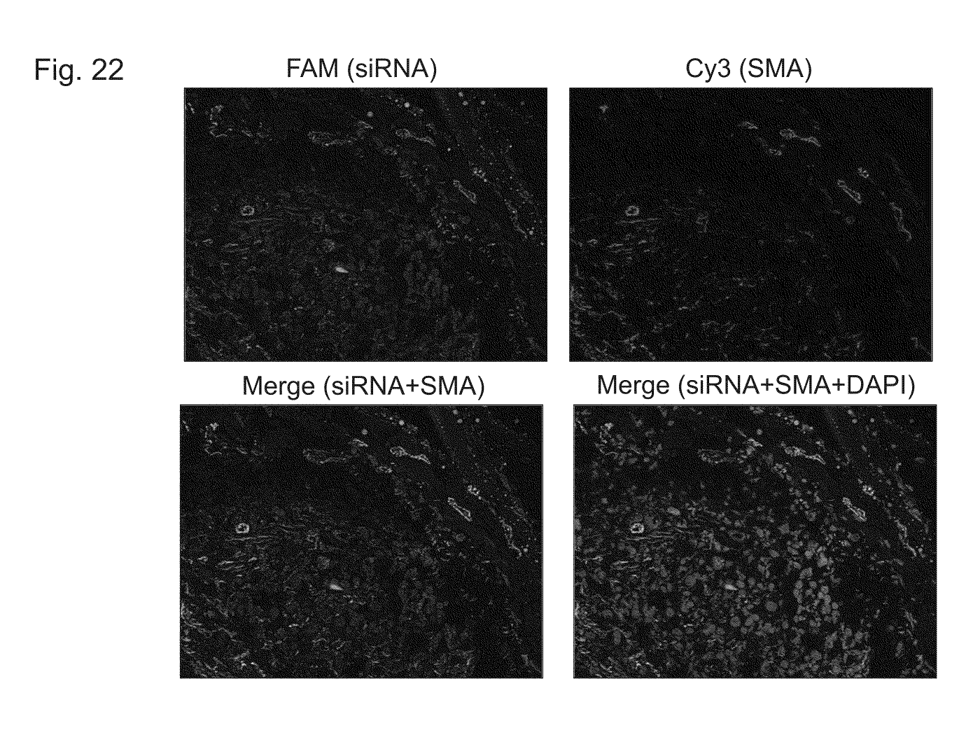

NOD-scid mice (6 weeks old, female, n=8, purchased from Sankyo Labo Service Corporation) were subcutaneously inoculated with stomach cancer cell line KATO-III at 2×10⁶ cells, thus making tumor-bearing mice. On the 28th day after inoculation, VA-bound liposome (VA-lip-siRNA-FAM) or liposome containing no VA (lip-siRNA-FAM) used in Example 5 were administered via the tail vein at doses of 200 nmol of VA, 100 nmol of lip, and 100 μg of siRNA. In this VA-bound liposome, part of the VA was already exposed on the surface of liposome when administered. 24 hours after administration, tumor tissue was collected, a tissue specimen was prepared, this was stained with DAPI (Molecular Probe) and Cy3-labeled anti α-SMA antibody, and the localization of siRNA was analyzed. The results are shown in FIGS. 21 and 22.

As is clear from FIG. 21, in the liposome containing no VA, in spite of the presence of CAFs in the tissue shown by the red color due to Cy3, there was hardly any siRNA shown by the green color due to FAM, whereas in the VA-bound liposome, colocalization of CAF and siRNA was observed.

### Example 12

### In Vivo VA-lip-DNR Antitumor Activity

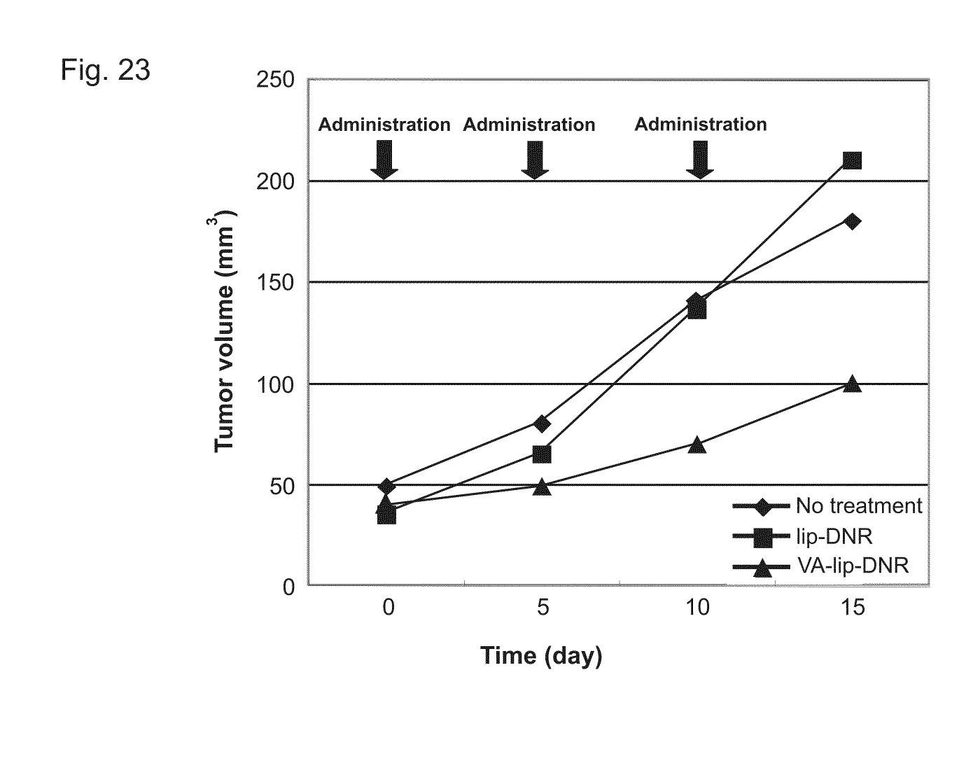

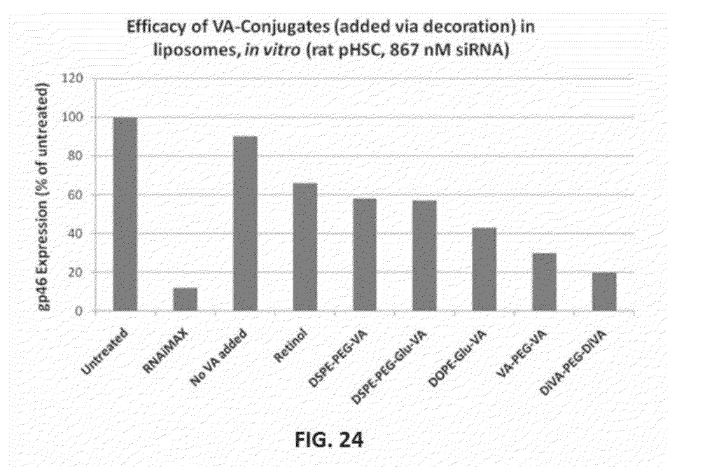

Nude mice (6 weeks old, female, n=10, purchased from Sankyo Labo Service Corporation) were subcutaneously inoculated with colon cancer cell line M7609 cells at 2×10⁶ cells, thus giving tumor-bearing mice. From the 14th day after inoculation, VA-lip-DNR or lip-DNR was administered via the tail vein twice a week at a dose 1/40 (0.05 μg per g weight of the mouse) of the normal anticancer administration amount of DaunoXome®. In this VA-lip-DNR, part of the VA was already exposed on the surface of liposome when administered. The change in volume of the tumor after starting administration is shown in FIG. 23. It can be seen from this figure that the drug supported on the VA-bound carrier remarkably suppressed the growth of the tumor.

### Example 13

### Synthesis of DOPE-Glu-VA

### Preparation of (Z)-(2R)-3-(((2-(5-(((2E,4E,6E,8E)-3,7-dimethyl-9-(2,6,6-trimethylcyclohex-1-en-1-yl)nona-2,4,6,8-tetraen-1-yl)oxy)-5-oxopentanamido)ethoxy)(hydroxy)phosphoryl)oxy)propane-1,2-diyl dioleate (DOPE-Glu-VA)

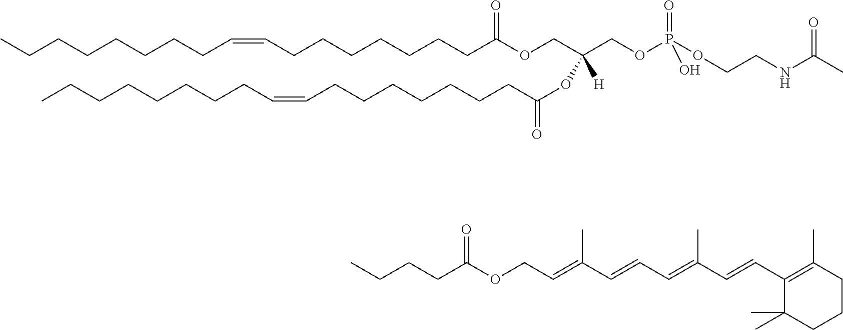

### Preparation of Intermediate 1: 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid

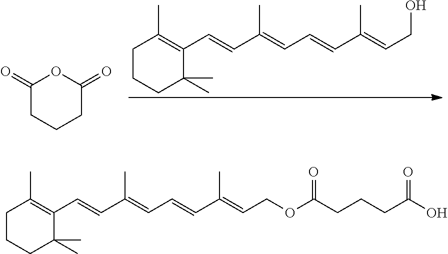

Glutaric anhydride (220 mg, 1.93 mmol) and retinol (500 mg, 1.75 mmol) were dissolved in dichloromethane (5 mL) in an amber-colored vial. Triethylamine (513 μl, 3.68 mmol) was added and the vial was flushed with argon. Reaction mixture was allowed to stir at room temperature for 4 hours. The material was concentrated and purified by silica gel chromatography with a dichloromethane/methanol gradient. Fractions were pooled and concentrated to yield yellowish oil (700 mg). The product was verified by NMR.

### Preparation of DOPE-Glu-VA: (Z)-(2R)-3-(((2-(5-(((2E,4E,6E,8E)-3,7-dimethyl-9-(2,6,6-trimethylcyclohex-1-en-1-yl)nona-2,4,6,8-tetraen-1-yl)oxy)-5-oxopentanamido)ethoxy)(hydroxy)phosphoryl)oxy)propane-1,2-diyl dioleate

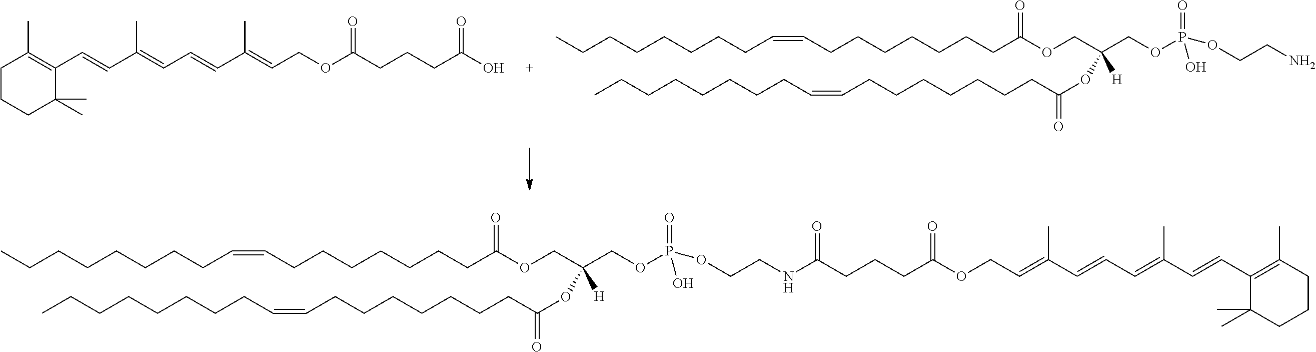 [SMILES: CC1=C(/C=C/C(C)=C/C=C/C(C)=C/COC(=O)CCCC(=O)O)C(C)(C)CCC1 ; CCCCCCCC/C=C\CCCCCCCC(=O)OC[C@H](COP(=O)(O)OCCN)OC(=O)CCCCCCC/C=C\CCCCCCCC ; CCCCCCCC/C=C\CCCCCCCC(=O)OC[C@H](COP(=O)(O)OCCNC(=O)CCCC(=O)OC/C=C(C)/C=C/C=C(C)/C=C/C1=C(C)CCCC1(C)C)OC(=O)CCCCCCC/C=C\CCCCCCCC]

1,2-dioleoyl-sn-glycero-3-phosphoethanolamine (500 mg, 0.672 mmol), N,N,N′,N′-Tetramethyl-O-(7-azabenzotriazol-1-yl)uronium hexafluorophosphate (306.5 mg, 0.806 mmol) and 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid (269 mg, 0.672 mmol) was dissolved in chloroform/DMF (10 mL, 1:1 mixture) in an amber-colored vial flushed with argon and N,N-Diisopropylethylamine (300 μL, 1.68 mmol) was added. Reaction mixture was allowed to stir overnight at room temperature. The reaction mixture was concentrated and then purified by silica gel chromatography using a dichloromethane/methanol gradient. The fractions were pooled and concentrated to yield yellowish oil (460 mg, 61%). Verified product by NMR. ¹H NMR (400 MHz), δ_{H}: 8.6 (d, 1H), 8.27 (d, 1H), 6.57-6.61 (dd, 1H), 6.08-6.25 (m, 4H), 5.57 (t, 1H), 5.30-5.34 (m, 4H), 5.18 (m, 1H), 4.68-4.70 (d, 2H), 4.28-4.35 (m, 1H), 4.05-4.15 (m, 1H), 3.81-3.97 (m, 4H), 3.52-3.62 (m, 1H), 3.35-3.45 (m, 2H), 2.95-3.05 (m, 1H), 2.33-2.35 (t, 3H), 2.2-2.3 (m, 7H), 1.9-2.05 (m, 17H), 1.85 (s, 3H), 1.69 (s, 3H), 1.5-1.65 (m, 6H), 1.4-1.5 (m, 2H), 1.18-1.38 (m, ˜40H), 1.01 (s, 3H), 0.84-0.88 (m, 12H).

### Example 14

### DOPE-Glu-NH-VA

### Preparation of (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy)(hydroxy)-phosphoryl)oxy)propane-1,2-diyl dioleate (DOPE-Glu-NH-VA)

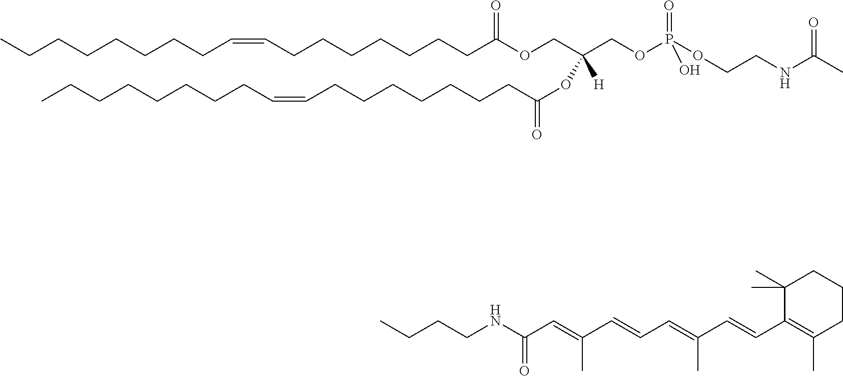

### Preparation of Intermediate 1: (Z)-(2R)-3-(((2-(4-aminobutanamido)ethoxy)-(hydroxy)phosphoryl)oxy)propane-1,2-diyl dioleate

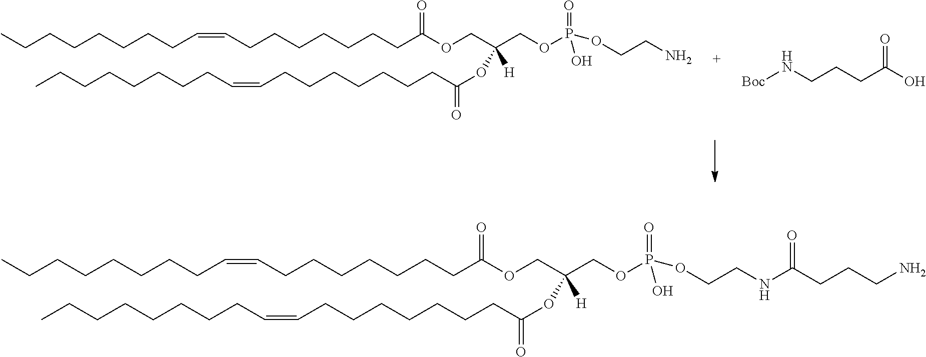 [SMILES: CCCCCCCC/C=C\CCCCCCCC(=O)OC[C@H](COP(=O)(O)OCCN)OC(=O)CCCCCCC/C=C\CCCCCCCC ; CC(C)(C)OC(=O)NCCCC(=O)O ; CCCCCCCC/C=C\CCCCCCCC(=O)OC[C@H](COP(=O)(O)OCCNC(=O)CCCN)OC(=O)CCCCCCC/C=C\CCCCCCCC]

1,2-Dioleoyl-sn-glycero-3-phosphoethanolamine (2500 mg, 3.36 mmol), Boc-GABA-OH (751 mg, 3.70 mmol) and N,N,N′,N′-Tetramethyl-O-(7-azabenzotriazol-1-yl)uronium hexafluorophosphate (1531 mg, 4.03 mmol) were dissolved in a DMF/chloroform (25 mL, 1:1 mixture). N,N-Diisopropylethylamine (880 μL, 5.05 mmol) was added and the mixture was allowed to stir at room temperature overnight under a blanket of argon. The reaction mixture was diluted with ˜200 mL H₂O and product was extracted with dichloromethane (3×100 ml). The product was washed with ˜75 mL pH 4.0 PBS buffer, dried organics with sodium sulfate, filtered and concentrated. Material was then purified via silica gel chromatography with a dichloromethane/methanol gradient, and concentrated to yield colorless oil (2.01 g, 64%). The product was verified by NMR. Material was then taken up in 30 mL of 2 M HCl/diethyl ether. Reaction was allowed to stir at room temperature in a H₂O bath. After 2 hours, the solution was concentrated to yield (Z)-(2R)-3-(((2-(4-aminobutanamido)ethoxy)(hydroxy)phosphoryl)oxy)propane-1,2-diyl dioleate.

### Preparation of DOPE-Glu-NH-VA: (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy) (hydroxy)phosphoryl)oxy)propane-1,2-diyl dioleate

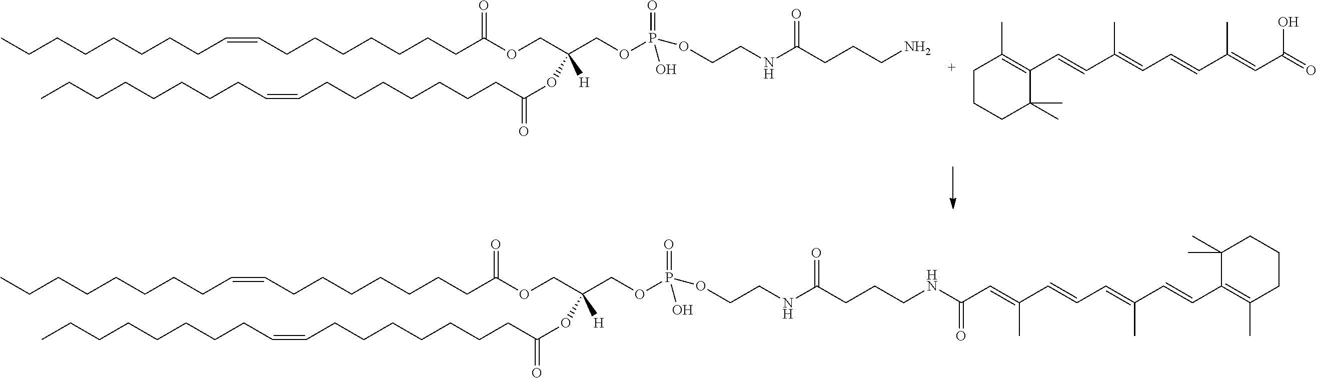 [SMILES: CCCCCCCC/C=C\CCCCCCCC(=O)OC[C@H](COP(=O)(O)OCCNC(=O)CCCN)OC(=O)CCCCCCC/C=C\CCCCCCCC ; CC1=C(/C=C/C(C)=C/C=C/C(C)=C/C(=O)O)C(C)(C)CCC1 ; CCCCCCCC/C=C\CCCCCCCC(=O)OC[C@H](COP(=O)(O)OCCNC(=O)CCCNC(=O)/C=C(C)/C=C/C=C(C)/C=C/C1=C(C)CCCC1(C)C)OC(=O)CCCCCCC/C=C\CCCCCCCC]

(Z)-(2R)-3-(((2-(4-aminobutanamido)ethoxy)(hydroxy)phosphoryl)oxy) propane-1,2-diyl dioleate (1200 mg, 1.45 mmol), retinoic acid (500 mg, 1.66 mmol) and N,N,N′,N′-Tetramethyl-O-(7-azabenzotriazol-1-yl)uronium hexafluorophosphate (689 mg, 1.81 mmol) was suspended in DMF/chloroform (10 mL, 1:1 mixture). N,N-Diisopropylethylamine (758 μL, 4.35 mmol) was added. The round bottom flask was flushed with argon and covered with aluminum foil. Reaction mixture was stirred at room temperature for 4 hours, partitioned in dichloromethane (75 mL) and H₂O (75 mL), extracted with dichloromethane, dried (sodium sulfate), filtered and concentrated. Purification by silica gel chromatography using a dichloromethane/methanol gradient yielded (Z)-(2R)-3-(((2-(4-((2E,4E,6E,8E)-3,7-dimethyl-9-(2,6,6-trimethylcyclohex-1-en-1-yl)nona-2,4,6,8-tetraenamido)butanamido)ethoxy)(hydroxy)phosphoryl)oxy)propane-1,2-diyl dioleate (292 mg, 18%). The product was characterized by LCMS & NMR. ¹H NMR (400 MHz), δ_{H}: 8.55 (s, 1H), 8.2 (d, 1H), 7.3 (s, 1H), 6.6 (dd, 1H), 6.10-6.27 (m, 5H), 5.5 (t, 1H), 5.31 (s, 4H), 5.1-5.2 (m, 2H), 4.68 (d, 2H), 4.3 (d, 2H), 4.1 (m, 2H), 3.9 (m, 8H), 3.58 (q, 4H), 3.4 (s, 4H), 3.0 (q, 4H), 2.33-2.35 (t, 3H), 2.2-2.3 (m, 7H), 1.9-2.05 (m, 17H), 1.85 (s, 3H), 1.69 (s, 3H), 1.5-1.65 (m, 6H), 1.4-1.5 (m, 2H), 1.18-1.38 (m, ˜40H), 1.01 (s, 3H), 0.84-0.88 (m, 12H). MS: m/z 1112.44 (M+H⁺).

### Example 15

### DSPE-PEG550-VA

### Preparation of (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate (DSPE-PEG550-VA)

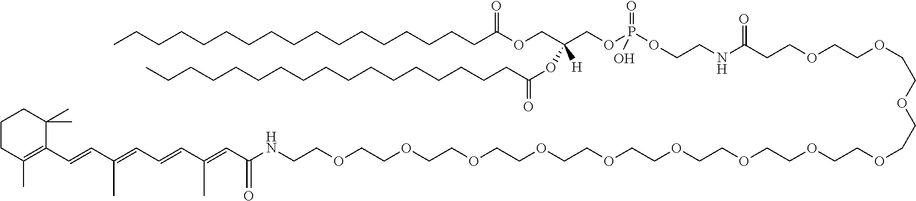 [SMILES: CCCCCCCCCCCCCCCCCC(=O)OC[C@H](COP(=O)(O)OCCNC(=O)CCOCCOCCOCCOCCOCCOCCOCCOCCOCCOCCOCCOCCNC(=O)/C=C(C)/C=C/C=C(C)/C=C/C1=C(C)CCCC1(C)C)OC(=O)CCCCCCCCCCCCCCCCC]

### Preparation of Intermediate 1: (2R)-3-((2,2-dimethyl-4,44-dioxo-3,8,11,14,17,20,23,26,29,32,35,38,41-tridecaoxa-5,45-diazaheptatetracontan-47-yl)oxy)(hydroxy)phosphoryl)oxy)propane-1,2-diyl distearate

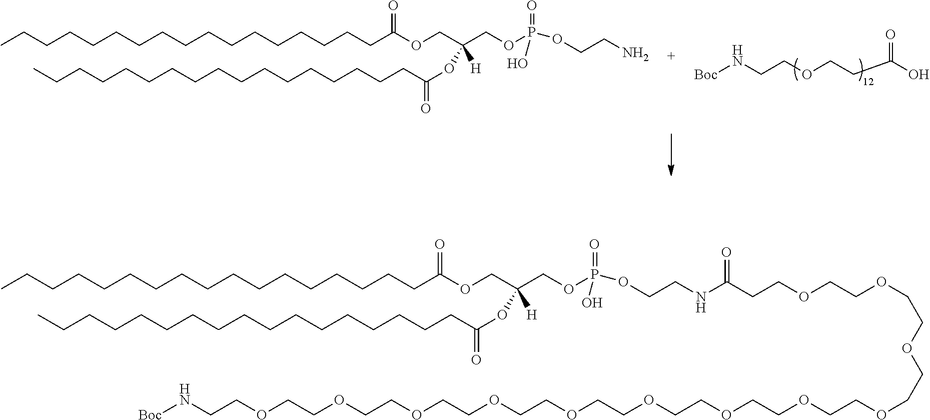

1,2-Distearoyl-sn-glycero-3-phosphoethanolamine (200 mg, 0.267 mmol), t-Boc-N-amido-dPEG₁₂-acid (211 mg, 0.294 mmol) and N,N,N′,N′-Tetramethyl-O-(7-azabenzotriazol-1-yl)uronium hexafluorophos-phate (122 mg, 0.320 mmol) were dissolved in a chloroform/methanol/H₂O (6 mL, 65:35:8) in a 20 mL scintillation vial flushed with argon. N,N-Diisopropylethylamine (116 μL, 0.668 mmol) was added. Reaction was allowed to stir at 25° C. for 4 hours and concentrated. Material was then purified via silica gel chromatography with a dichloromethane/methanol gradient to yield (2R)-3-((((2,2-dimethyl-4,44-dioxo-3,8,11,14,17,20,23,26,29,32,35,38,41-tridecaoxa-5,45-diazaheptatetracontan-47-yl)oxy)(hydroxy)phosphoryl)oxy)propane-1,2-diyl distearate as an oil (252 mg, 65%).

### Preparation of DSPE-PEG550-VA: (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate

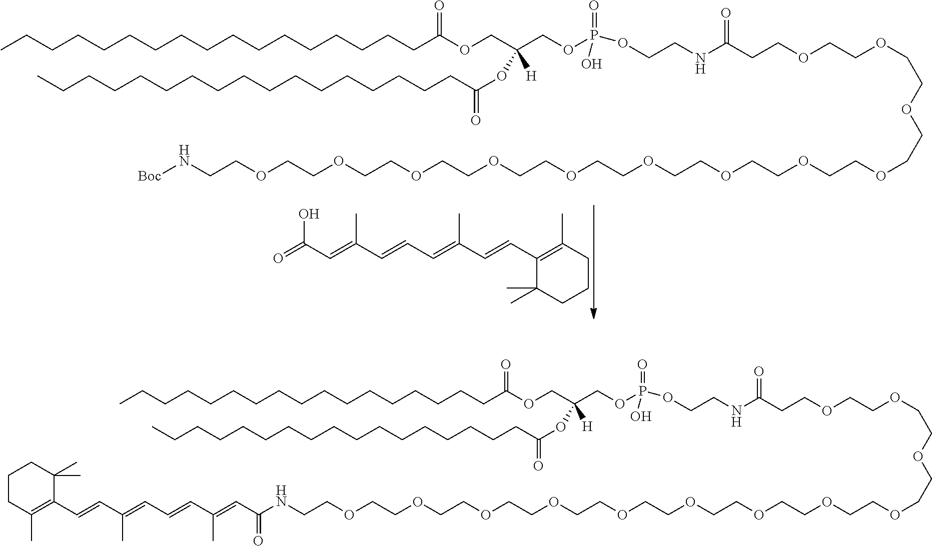

(2R)-3-((((2,2-dimethyl-4,44-dioxo-3,8,11,14,17,20,23,26,29,32,35,38,41-tridecaoxa-5,45-diazaheptatetracontan-47-yl)oxy)(hydroxy)phosphoryl)oxy)propane-1,2-diyl distearate (252 mg, 0.174 mmol) was dissolved in diethyl ether (5 mL). Reaction was placed in a H₂O bath at room temperature. 2 M HCl/diethyl ether (2 mL, 4 mmol) was added and the mixture was allowed to stir for approximately 1 hour. Afterwards, solvent and excess HCl were removed in vacuo. Suspended material in 2 mL N,N-Dimethylformamide in a round bottom flask flushed with argon. Retinoic acid (57.5 mg, 0.191 mmol), N,N,N′,N′-Tetramethyl-O-(7-azabenzotriazol-1-yl)uronium hexafluorophosphate (79 mg, 0.209 mmol) and N,N-Diisopropylethylamine (106 μL, 0.609 mmol) were added. The material did not fully dissolve thus added more chloroform/methanol/H₂O (1 mL, 65:35:8 v:v:v mixture) to get reaction homogeneous. After 3.5 hours, the reaction mixture was concentrated. Material was then purified via silica gel chromatography with a dichloromethane/methanol gradient to yield (2R)-3-(((((45E,47E,49E,51E)-46,50-dimethyl-4,44-dioxo-52-(2,6,6-trimethylcyclohex-1-en-1-yl)-7,10,13,16,19,22,25,28,31,34,37,40-dodecaoxa-3,43-diazadopentaconta-45,47,49,51-tetraen-1-yl)oxy)(hydroxy)phosphoryl)oxy)propane-1,2-diyl distearate as a tan solid (210 mg, 74%). Verified product by NMR & LCMS. ¹H NMR (400 MHz), δ_{H}: 8.6 (s, 1H), 8.25 (d, 1H), 6.8-6.9 (dd, 1H), 6.3-6.4 (m, 1H), 6.12-6.25 (dd, 5H), 5.71 (s, 1H), 5.18 (m, 2H), 4.33 (dd, 2H), 4.13 (m, 2H), 3.95 (m, 2H), 3.74 (m, 8H), 3.63 (s, ˜48H), 3.0 (q, 2H), 2.5 (t, 3H), 2.35 (s, 3H), 2.25 (t, 8H), 1.97 (m, 7H), 1.7 (3, 3H), 1.5 (m, 2H), 1.36 (m, 1214), 1.23 (m, ˜56H), 1.01 (s, 6H), 0.86 (t, 12H). MS: m/z 1630.28 (M+H⁺).

### Example 16

### DSPE-PEG2000-Glu-VA

### Preparation of DSPE-PEG2000-Glu-VA

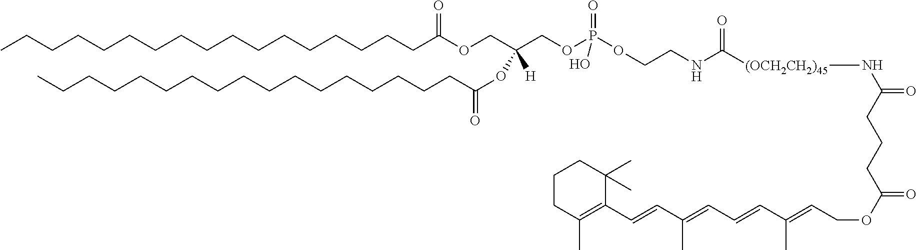

### Preparation of Intermediate 1: 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid

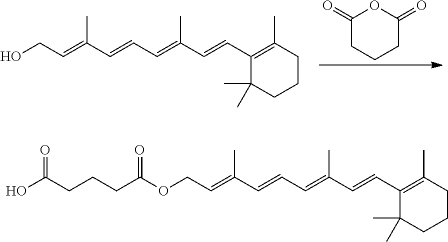 [SMILES: CC1=C(/C=C/C(C)=C/C=C/C(C)=C/CO)C(C)(C)CCC1 ; O=C1CCCC(=O)O1 ; CC1=C(/C=C/C(C)=C/C=C/C(C)=C/COC(=O)CCCC(=O)O)C(C)(C)CCC1]

Glutaric anhydride (115 mg, 1.01 mmol) and retinol (240 mg, 0.838 mmol) were dissolved in dichloromethane (3 mL) in an amber-colored vial. Triethylamine (257 μl, 1.84 mmol) was added and the vial was flushed with argon. Reaction was allowed to stir at room temperature overnight. The reaction mixture was concentrated and then purified via silica gel chromatography with a dichloromethane/methanol gradient to yield 5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid as a yellowish oil (700 mg, 78%). Material characterized by NMR.

### Preparation of DSPE-PEG2000-Glu-VA

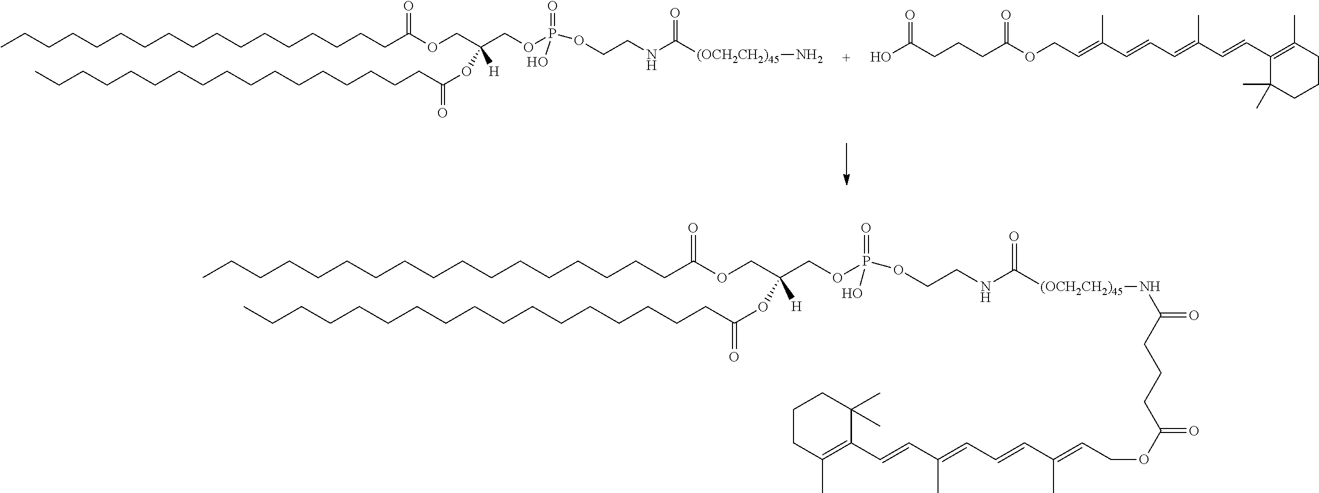

5-(((2E,4E,6E,8E)-3,7-dimethyl-9-(2,6,6-trimethylcyclohex-1-en-1-yl)nona-2,4,6,8-tetraen-1-yl)oxy)-5-oxopentanoic acid (43 mg, 0.108 mmol), DSPE-PEG2000—NH₂ (250 mg, 0.090 mmol) and N,N,N′,N′-tetramethyl-O-(7-azabenzotriazol-1-yl)uronium hexafluorophosphate (45 mg, 0.117 mmol) were dissolved in N,N-dimethylformamide (2 mL) in an amber-colored scintillation vial flushed with argon gas. N,N-diisopropylethylamine (47 μL, 0.270 mmol) was added and the reaction was allowed to stir overnight at room temperature, then purified via silica gel chromatography with a dichloromethane/methanol gradient to yield yellowish oil (59 mg, 20.7%). Verified product by NMR. ¹H NMR (400 MHz), δ_{H}: 706 (m, 1H), 6.59-6.66 (dd, 1H), 6.06-6.30 (m 5H), 5.56-5.60 (t, 1H), 5.17-5.23 (m, 2H), 4.35-4.42 (dd, 2H), 4.12-4.25 (m, 5H), 3.96-3.97 (m, 6H), 3.79-3.81 (t, 1H), 3.66 (m, ˜180H), 3.51-3.58 (m, 2H), 3.4-3.48 (m, 4H), 3.3-3.38 (m, 2H), 2.25-2.45 (m, 14H), 1.5-2.0 (m, 15H), 1.23-1.32 (m, ˜56H), 1.01 (s, 3H), 0.85-0.88 (t, 12H).

### Example 17

### DOPE-Gly₃-VA

### Preparation of (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)propane-1,2-diyl dioleate DOPE-Gly₃-VA)

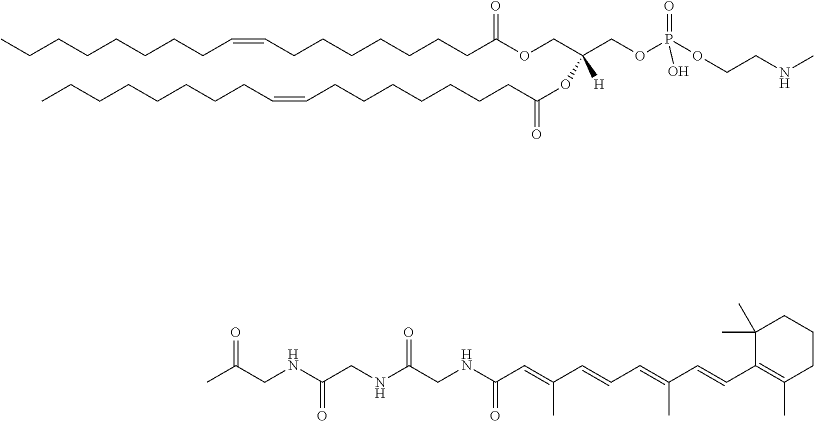

### Preparation of Intermediate 1: (Z)-(2R)-3-(((2-(2-(2-(2-aminoacetamido)acetamido)acetamido)ethoxy)(hydroxy)phosphoryl)oxy)propane-1,2-diyl dioleate

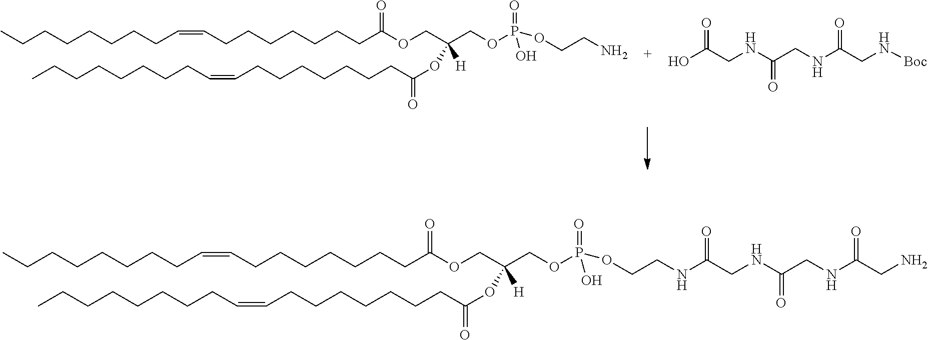 [SMILES: CCCCCCCC/C=C\CCCCCCCC(=O)OC[C@H](COP(=O)(O)OCCN)OC(=O)CCCCCCC/C=C\CCCCCCCC ; CC(C)(C)OC(=O)NCC(=O)NCC(=O)NCC(=O)O ; CCCCCCCC/C=C\CCCCCCCC(=O)OC[C@H](COP(=O)(O)OCCNC(=O)CNC(=O)CNC(=O)CN)OC(=O)CCCCCCC/C=C\CCCCCCCC]

Boc-Gly-Gly-Gly-OH (382 mg, 1.34 mmol) and N,N,N′,N′-Tetramethyl-O-(7-azabenzotriazol-1-yl)uronium hexafluorophosphate (532 mg, 1.4 mmol) were dissolved in DMF (5 mL). N,N-Diisopropylethylamine (488 μL, 2.8 mmol) was added and the mixture was allowed to stir at room temperature for 10-15 minutes. Afterwards, a solution of 1,2-dioleoyl-sn-glycero-3-phosphoethanolamine (833 mg, 1.12 mmol) in chloroform (5 mL) was added and the reaction vessel was flushed with argon. After 16 hours at room temperature, the reaction mixture was concentrated and partitioned between dichloromethane (50 mL) and H₂O (50 mL), extracted with dichloromethane (3×50 mL), dried with sodium sulfate, filtered and concentrated. Material was purified via silica gel chromatography using a dichloromethane/methanol gradient to yield colorless oil residue. To this, 2 M HCl/Diethyl Ether (5 mL) was added and the reaction mixture was allowed to stir in a H₂O bath for approximately 2 hours. The reaction mixture was concentrated and the residue was taken up in dichloromethane (75 mL), washed with saturated sodium bicarbonate solution (75 mL), extracted product with dichloromethane (3×75 mL), dried with sodium sulfate, filtered and concentrated to yield (Z)-(2R)-3-(((2-(2-(2-(2-aminoacetamido)acetamido)acetamido)ethoxy)(hydroxy)phosphoryl)oxy)propane-1,2-diyl dioleate as a semi-solid (765 mg, 90%). Verified by NMR.

### Preparation of DOPE-Gly₃-VA: (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)propane-1,2-diyl dioleate

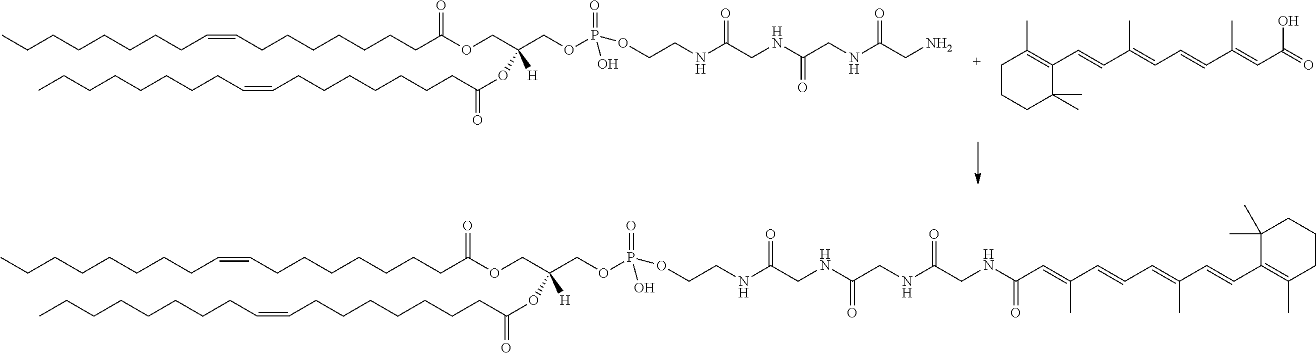 [SMILES: CCCCCCCC/C=C\CCCCCCCC(=O)OC[C@H](COP(=O)(O)OCCNC(=O)CNC(=O)CNC(=O)CN)OC(=O)CCCCCCC/C=C\CCCCCCCC ; CC1=C(/C=C/C(C)=C/C=C/C(C)=C/C(=O)O)C(C)(C)CCC1 ; CCCCCCCC/C=C\CCCCCCCC(=O)OC[C@H](COP(=O)(O)OCCNC(=O)CNC(=O)CNC(=O)CNC(=O)/C=C(C)/C=C/C=C(C)/C=C/C1=C(C)CCCC1(C)C)OC(=O)CCCCCCC/C=C\CCCCCCCC]

(Z)-(2R)-3-(((2-(2-(2-(2-aminoacetamido)acetamido)acetamido)ethoxy) (hydroxy)phosphoryl)oxy)propane-1,2-diyl dioleate (765 mg, 0.836 mmol), retinoic acid (301 mg, 1.00 mmol), and N,N,N′,N′-Tetramethyl-O-(7-azabenzotriazol-1-yl)uronium hexafluorophosphate (413 mg, 1.09 mmol) were suspended in N,N-Dimethylformamide (5 mL). N,N-Diisopropylethylamine (437 μL, 2.51 mmol) was added and the reaction vessel was flushed with argon gas. Added chloroform (5 mL) to aid in the solvation of materials. Reaction was allowed to stir for ˜4 hours at room temperature in a round bottom flask covered with aluminum foil. Partitioned material between H₂O (100 mL) and dichloromethane (100 mL). Extracted with dichloromethane (3×100 mL), dried with sodium sulfate, filtered and concentrated. Material was then purified via silica gel chromatography using a dichloromethane/methanol gradient to yield (Z)-(2R)-3-(((((14E,16E,18E,20E)-15,19-dimethyl-4,7,10,13-tetraoxo-21-(2,6,6-trimethylcyclohex-1-en-1-yl)-3,6,9,12-tetraazahenicosa-14,16,18,20-tetraen-1-yl)oxy)(hydroxy)phosphoryl)oxy)propane-1,2-diyl dioleate as an orange oil (704 mg, 70%). Verified product by LCMS & NMR. ¹H NMR (400 MHz), δ_{H}: 6.90 (t, 1H), 6.21 (q, 2H), 6.08-6.12 (d, 2H), 5.83 (s, 1H), 5.31 (s, 4H), 5.30 (s, 2H), 4.37 (d, 1H), 4.15 (m, 1H), 3.91 (m, 8H), 3.59 (m, 2H), 3.29 (m, 2H), 3.01 (m, 2H), 2.28 (m, 6H), 1.95-1.98 (m, 12H), 1.44 (s, 3H), 1.5-1.6 (m, 2H), 1.44 (m, 6H), 1.24 (m, ˜48H), 1.00 (s, 6H), 0.86 (t, 3H). MS: m/z 1198.42 (M+H⁺).

### Example 18

### VA-PEG-VA

### Preparation of N1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide (VA-PEG-VA)

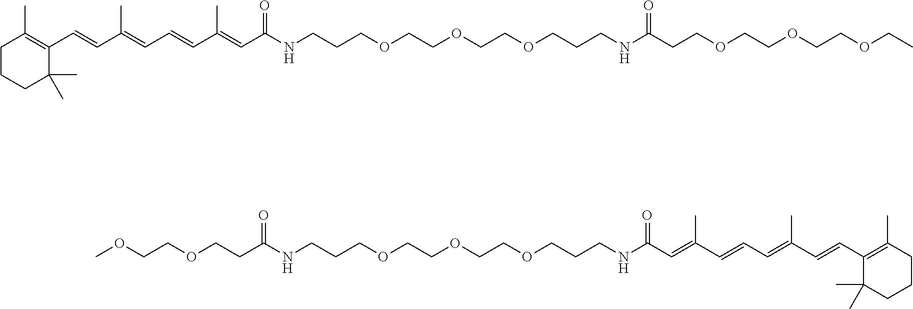

### Preparation of VA-PEG-VA: N1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide

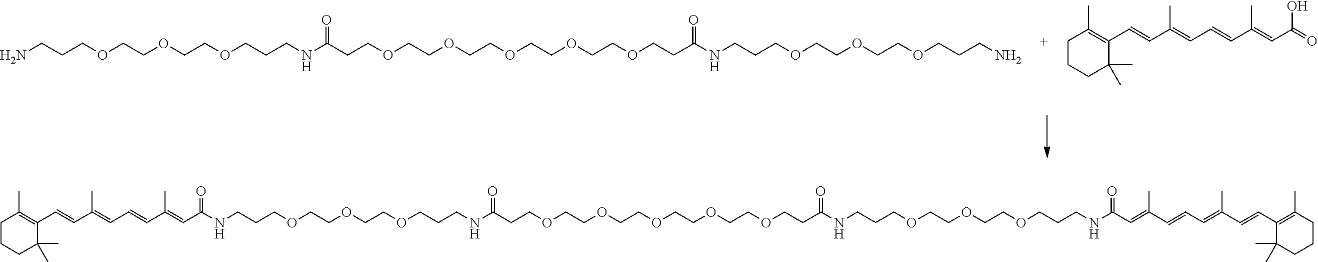 [SMILES: NCCCOCCOCCOCCCNC(=O)CCOCCOCCOCCOCCOCCC(=O)NCCCOCCOCCOCCCN ; CC1=C(/C=C/C(C)=C/C=C/C(C)=C/C(=O)O)C(C)(C)CCC1 ; CC1=C(/C=C/C(C)=C/C=C/C(C)=C/C(=O)NCCCOCCOCCOCCCNC(=O)CCOCCOCCOCCOCCOCCC(=O)NCCCOCCOCCOCCCNC(=O)/C=C(C)/C=C/C=C(C)/C=C/C2=C(C)CCCC2(C)C)C(C)(C)CCC1]

Retinoic acid (2913 mg, 9.70 mmol), N,N,N′,N′-Tetramethyl-O-(7-azabenzotriazol-1-yl)uronium hexafluorophosphate (3992 mg, 10.50 mmol) and diamido-dPEG₁₁-diamine (3000 mg, 4.04 mmol) were suspended in N,N-dimethylformamide (10 mL). N,N-Diisopropylethylamine (4222 μL, 24.24 mmol) was added and the vessel was flushed with argon. Reaction was allowed to stir at room temperature overnight in a round bottom flask covered with aluminum foil. Next day, partitioned material between ethyl acetate (125 mL) and water (125 mL). Extracted with ethyl acetate (3×125 mL), dried with sodium sulfate, filtered and concentrated. Material was then purified via silica gel chromatography with a dichloromethane/methanol gradient. Pooled fractions and concentrated to yield yellow oil (2900 mg, 54.9%). Verified product by LCMS & NMR. ¹H NMR (400 MHz), δ_{H}: 7.1 (s, 2H), 6.87 (t, 2H), 6.51 (t, 2H), 6.12-6.20 (dd, 8H), 5.66 (s, 2H), 3.6-3.8 (m, ˜44H), 3.4 (q, 4H), 3.3 (q, 4H), 2.46 (t, 4H), 2.32 (s, 6H), 1.9-2.05 (m, 10H), 1.7-1.85 (m, 15H), 1.6 (m, 4H), 1.3-1.5 (m, 6H), 1.01 (s, 12H). QTOF MS: m/z 1306 (M+H⁺).

### Example 19

### VA-PEG2000-VA

### Preparation of (2E,2′E,4E,4′E,6E,6′E,8E,8′E)-N,N′-(3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxatetracontahectane-1,140-diyl)bis(3,7-dimethyl-9-(2,6,6-trimethylcyclohex-1-en-1-yl)nona-2,4,6,8-tetraenamide) (VA-PEG2000-VA)

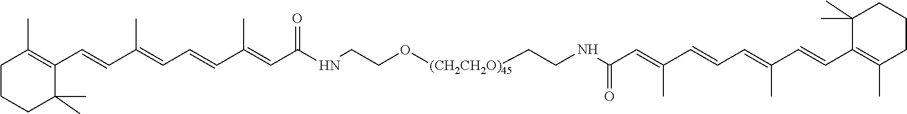

### Preparation of VA-PEG2000-VA: (2E,2′E,4E,4′E,6E,6′E,8E,8E)-N,N′-(3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxatetracontahectane-1,140-diyl)bis(3,7-dimethyl-9-(2,6,6-trimethylcyclohex-1-en-1-yl)nona-2,4,6,8-tetraenamide)

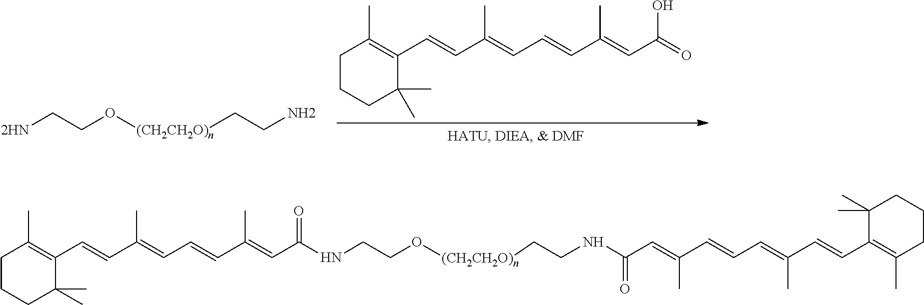

Retinoic acid (109 mg, 0.362 mmol), N,N,N′,N′-Tetramethyl-O-(7-azabenzotriazol-1-yl)uronium hexafluorophosphate (149 mg, 0.392 mmol) and amine-PEG_{2K}-amine (333 mg, 0.151 mmol) were suspended in N,N-Dimethylformamide (3 mL). N,N-Diisopropylethylamine (158 μL, 0.906 mmol) was added and the vessel was flushed with argon. Reaction was allowed to stir at room temperature overnight in a round bottom flask covered with aluminum foil. Next day, partitioned material between ethyl acetate (30 mL) and water (30 mL). Extracted with ethyl acetate (3×30 mL), dried with sodium sulfate, filtered and concentrated. Material was then purified via silica gel chromatography with a dichloromethane/methanol gradient. Pooled fractions and concentrated to yield (2E,2′E,4E,4′E,6E,6′E,8E,8′E)-N,N′-(3,6,9,12,15,18,21,24,27,30,33,36,39,42,45,48,51,54,57,60,63,66,69,72,75,78,81,84,87,90,93,96,99,102,105,108,111,114,117,120,123,126,129,132,135,138-hexatetracontaoxatetracontahectane-1,140-diyl)bis(3,7-dimethyl-9-(2,6,6-trimethylcyclohex-1-en-1-yl)nona-2,4,6,8-tetraenamide) as a yellow oil (97 mg, 23%). Verified product by LCMS & NMR. ¹H NMR (400 MHz), δ_{H}: 6.85-6.92 (t, 2h), 6.20-6.32 (M, 6H), 6.08-6.12 (d, 4H), 5.72 (s, 2H), 3.55-3.70 (m, ˜180H), 3.4-3.5 (m, 4H), 2.79 (m, 4H), 2.78 (s, 61-1), 2.33 (s, 6H), 2.05 (m, 41-1), 1.97 (s, 6H), 1.80 (m, 2H), 1.79 (s, 6H), 1.69 (s, 6H), 1.60 (m, 4H), 1.45 (m, 4H), 1.01 (s, 12H). QTOF MS: m/z 2651 (M+H⁺).

### Example 20

### DSPE-PEG2000-VA

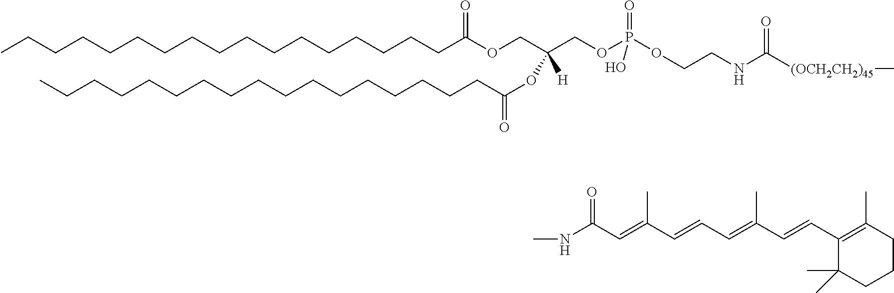

### Preparation of DSPE-PEG2000-VA

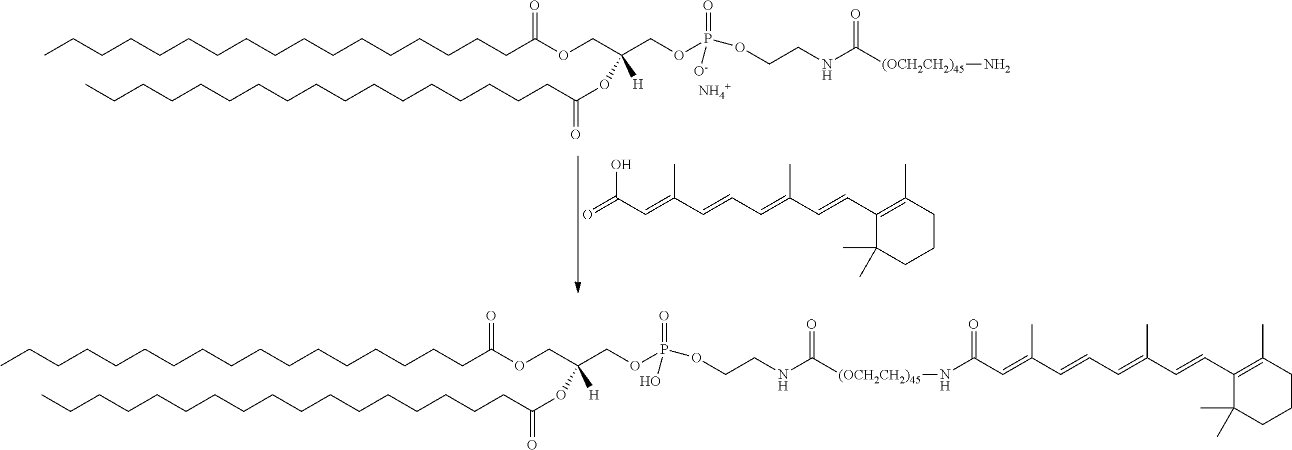

DSPE-PEG2000-NH₂ (250 mg, 0.090 mmol), retinoic acid (33 mg, 0.108 mmol) and N,N,N′,N′-Tetramethyl-O-(7-azabenzotriazol-1-yl)uronium hexafluorophosphate (45 mg, 0.117 mmol) were dissolved in N,N-Dimethylformamide. N,N-Diisopropylethylamine (47 μL, 0.270 mmol) was added to the mixture. The amber colored scintillation vial was flushed with argon and allowed to stir 3 days at room temperature. Material was then purified silica gel chromatography using a dichloromethane/methanol gradient. Pooled fractions and concentrated to yield DSPE-PEG2000-VA as a yellow oil (245 mg, 89%). Verified product by NMR. ¹H NMR (400 MHz), δ_{H}: 6.86 (dd, 1H), 6.25 (m, 1H), 6.09-6.21 (dd, 4H), 5.71 (s, 1H), 5.1-5.2 (m, 1H), 4.3-4.4 (d, 1H), 4.1-4.2 (m, 3H), 3.85-4.0 (m, 4H), 3.8 (t, 11-1), 3.5-3.75 (m, ˜180H), 3.4-3.5 (m, 8H), 3.3 (m, 2H), 2.35 (s, 3H), 2.26 (m, 4H), 1.70 (s, 3H), 1.55-1.65 (m, 6H), 1.47 (m, 2H), 1.23 (s, ˜60H), 1.01 (s, 611), 0.85 (t, 61-1).

### Example 21

### diVA-PEG-diVA, Also Known as “DIVA”

Preparation of N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide (diVA)

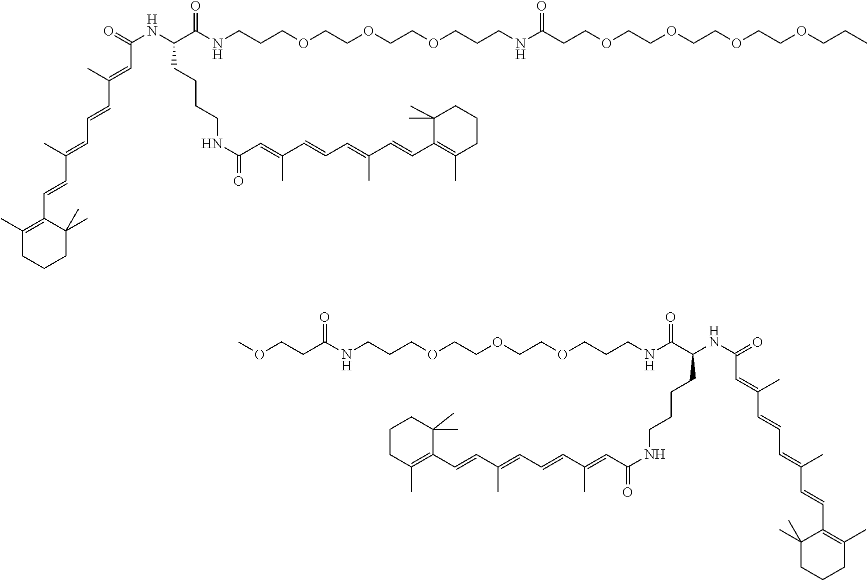

### Preparation of Intermediate 1: tetrabenzyl ((5S,57S)-6,22,40,56-tetraoxo-11,14,17,25,28,31,34,37, 45,48,51-undecaoxa-7,21,41,55-tetraazahenhexacontane-1,5,57,61-tetrayl)tetracarbamate, also known as Z-DiVA-PEG-DiVA-1N

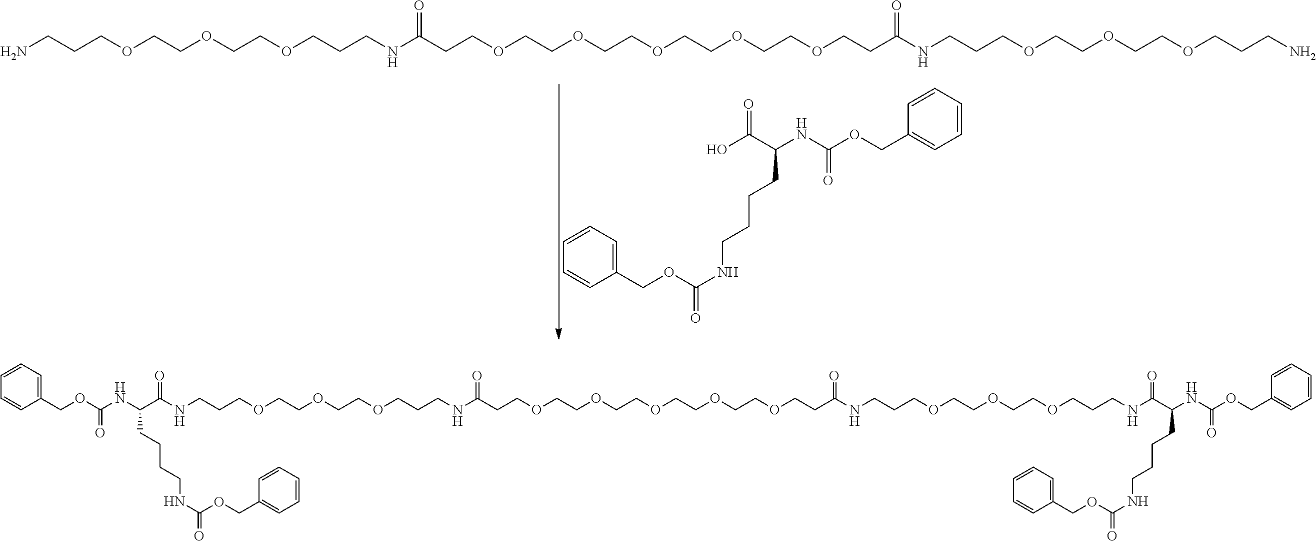 [SMILES: NCCCOCCOCCOCCCNC(=O)CCOCCOCCOCCOCCOCCC(=O)NCCCOCCOCCOCCCN ; O=C(NCCCC[C@H](NC(=O)OCc1ccccc1)C(=O)O)OCc1ccccc1 ; O=C(CCOCCOCCOCCOCCOCCC(=O)NCCCOCCOCCOCCCNC(=O)[C@H](CCCCNC(=O)OCc1ccccc1)NC(=O)OCc1ccccc1)NCCCOCCOCCOCCCNC(=O)[C@H](CCCCNC(=O)OCc1ccccc1)NC(=O)OCc1ccccc1]

A 1 L reaction flask cooled to 5-10° C. was purged with nitrogen and charged with dichloromethane (300 mL), d-PEG-11-diamine (Quanta lot EK1-A-1100-010, 50.0 g, 0.067 mol), Z-(L)-Lys(Z)-OH (61.5 g, 0.15 mol), and HOBt hydrate (22.5 g, 0.15 mol). 4-Methylmorpholine (4-MMP) (15.0 g, 0.15 mol) was added to the suspension and a light exothermic reaction was observed. A suspension of EDC hydrochloride (43.5 g, 0.23 mol) and 4-MMP (20.0 g, 0.20 mol) in dichloromethane (150 mL) was added over a period of 30 minutes, and moderate cooling was required in order to maintain a temperature of 20-23° C. The slightly turbid solution was stirred overnight at ambient temperature, and HPLC indicates completion of reaction. Deionized water (300 mL) was added and after having stirred for 10 minutes, a quick phase separation was observed. The aqueous phase was extracted with dichloromethane (150 mL)—with a somewhat slower phase separation. The combined organic extracts are washed with 6% sodium bicarbonate (300 mL) and dried with magnesium sulphate (24 g). Evaporation from a 40-45° C. water bath under reduced pressure gives 132 g of crude product. A solution of crude product (131 g) in 8% methanol in ethyl acetate in loaded onto a column of Silica Gel 60 (40-630, packed with 8% methanol in ethyl acetate. The column was eluted with 8% methanol in ethyl acetate (7.5 L). The fractions containing sufficiently pure product (5.00-7.25 L) was evaporated from a 45° C. water bath under reduced pressure and 83.6 g of purified product. A solution of purified product (83.6 g) in dichloromethane (200 mL) was loaded onto a column of Dowex 650 C (H⁺) (200 g), which has been washed with dichloromethane (250 mL). The column was eluted with dichloromethane (200 mL). The combined product containing fractions (300-400 mL) were dried with magnesium sulphate (14 g) and evaporated from a 45° C. water bath under reduced pressure to yield tetrabenzyl ((5 S,57S)-6,22,40,56-tetraoxo-11,14,17,25,28,31,34,37,45,48,51-undecaoxa-7,21,41,55-tetraazahenhexacontane-1,5,57,61-tetrayl)tetracarbamate, also known as Z-DiVA-PEG-DiVA-IN (77.9 g, HPLC purity 94.1%).

### Preparation of Intermediate 2: N1,N19-bis((S)-16,20-diamino-15-oxo-4,7,10-trioxa-14-azaicosyl)-4,7,10,13,16-pentaoxanonadecane-1,19-diamide, also known as DiVA-PEG-DiVA-IN

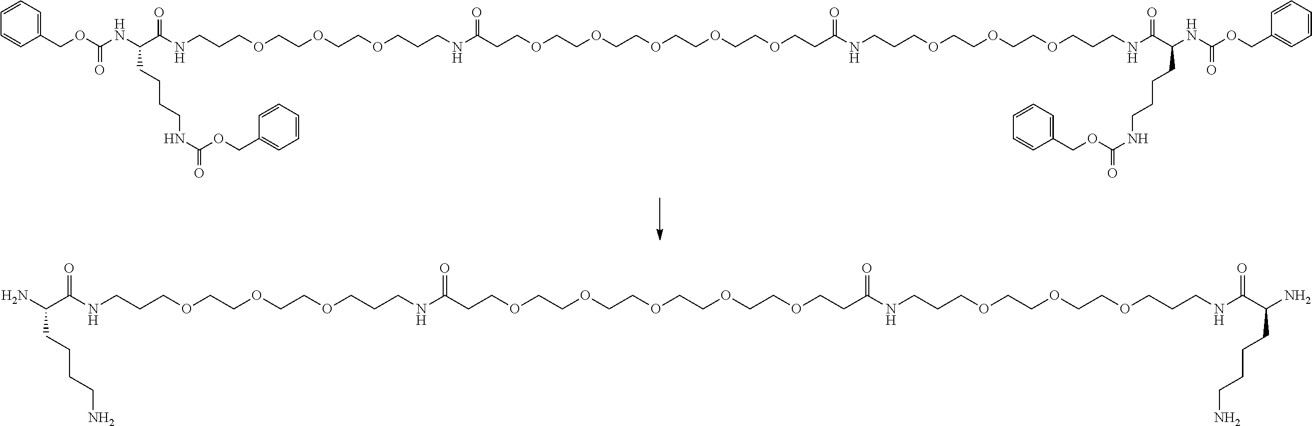 [SMILES: O=C(CCOCCOCCOCCOCCOCCC(=O)NCCCOCCOCCOCCCNC(=O)[C@H](CCCCNC(=O)OCc1ccccc1)NC(=O)OCc1ccccc1)NCCCOCCOCCOCCCNC(=O)[C@H](CCCCNC(=O)OCc1ccccc1)NC(=O)OCc1ccccc1 ; NCCCC[C@H](N)C(=O)NCCCOCCOCCOCCCNC(=O)CCOCCOCCOCCOCCOCCC(=O)NCCCOCCOCCOCCCNC(=O)[C@@H](N)CCCCN]

A 1 L reaction flask was purged with nitrogen and charged with methanol (600 mL) and Z-DiVA-PEG-DiVA-IN (92.9, 60.5 mmol). The mixture was stirred under nitrogen until a solution was obtained. The catalyst, 10% Pd/C/50% water (Aldrich, 10 g) was added. The mixture was evacuated, and then the pressure was equalized by nitrogen. The mixture was evacuated, and then the pressure was equalized by hydrogen. Ensuring a steady, low flow of hydrogen over the reaction mixture, the stirrer was started. Hydrogenation was continued in a flow of hydrogen for one hour. The system was then closed, and hydrogenation was continued at ˜0.1 bar for one hour. The mixture was evacuated and then re-pressurized to ˜0.1 bar with hydrogen. After another hour of hydrogenation, the mixture was evacuated and then re-pressurized to 0.1 bar with hydrogen. Stirring under hydrogen was continued for 15 hours after which time no starting material could be detected by HPLC. The mixture was evacuated, and then the pressure was equalized by nitrogen. The mixture was evacuated, and then the pressure was equalized by nitrogen. The reaction mixture was then filtered on a pad of celite 545. The filter cake was washed with methanol (100 mL). The combined filtrate was concentrated, finally at 45° C. and at a pressure of less than 50 mbar. Toluene (100 mL) was added and the resulting mixture was again concentrated finally at 45° C. and at a pressure of less than 40 mbar to yield N1,N19-bis((S)-16,20-diamino-15-oxo-4,7,10-trioxa-14-azaicosyl)-4,7,10,13,16-pentaoxanonadecane-1,19-diamide, also known as DiVA-PEG-DiVA-IN (63.4 g), as an oil that solidifies upon standing.

### Preparation of DiVA-PEG-DiVA: N1,N19-bis((S,23E,25E,27E,29E)-16-((2E,4E,6E,8E)-3,7-dimethyl-9-(2,6,6-trimethylcyclohex-1-en-1-yl)nona-2,4,6,8-tetraenamido)-24,28-dimethyl-15,22-dioxo-30-(2,6,6-tri-methylcyclohex-1-en-1-yl)-4,7,10-trioxa-14,21-diazatriaconta-23,25,27,29-tetraen-1-yl)-4,7,10,13,16-pentaoxanonadecane-1,19-diamide

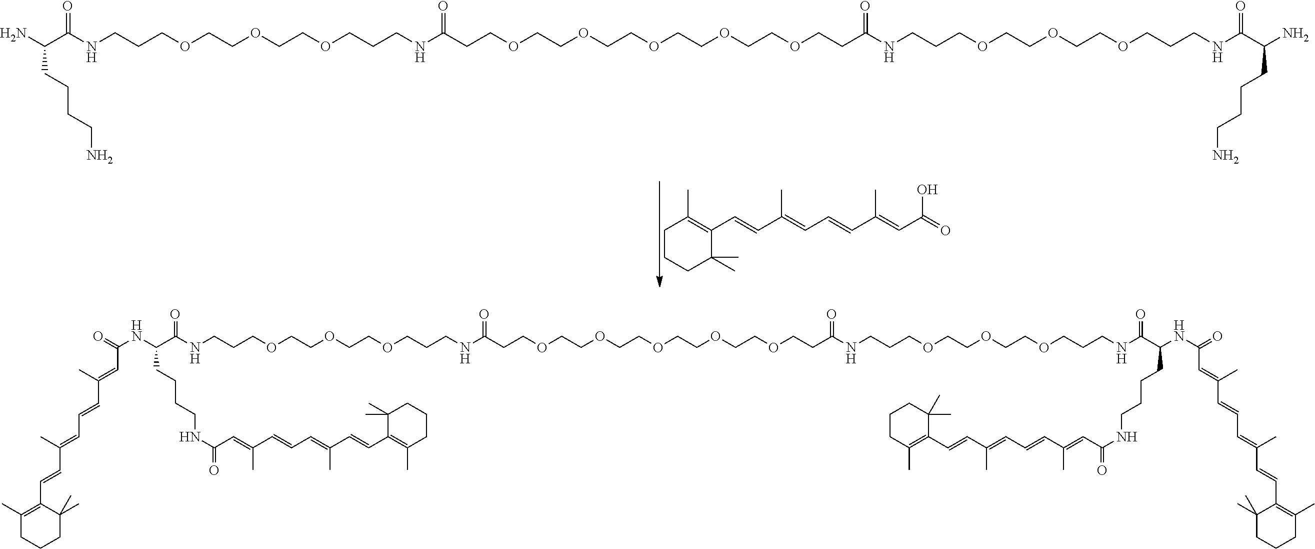

A 2 L reactor was filled with argon and charged with dichloromethane (500 mL), DiVA-PEG-DiVA-IN (52.3 g, 52.3 mmol), retinoic acid (70.6 g, 235 mmol) and 4-N,N-dimethylaminopyridine (2.6 g, 21.3 mmol). The mixture was stirred under argon until dissolved (˜20 minutes). Keeping the temperature of the reaction at 10-20° C., 1-ethyl-3-(3-dimethylaminopropyl) carbodiimide) (EDCI) (70.6 g, 369 mmol) was added portion wise over a period of 10-15 minutes (the reaction was slightly exothermic for the first 30-60 minutes). The reactor was covered with aluminium foil and the mixture was stirred at 18-21° C. for 15-20 hours. Butylated hydroxytoluene (BHT) (25 mg) was added and the reaction mixture was then poured onto aqueous 6% sodium hydrogen carbonate (500 mL) while keeping an argon atmosphere over the mixture. The organic phase was separated. The aqueous phase was washed with dichloromethane (50 mL). The combined organic phase was dried with of magnesium sulphate (150 g) under inert atmosphere and protected from light. The drying agent was filtered off (pressure filter preferred) and the filter cake was washed with dichloromethane (500 mL). The filtrate was concentrated by evaporation at reduced pressure using a water bath of 35-40° C. The oily residue was added toluene (150 mL) and evaporated again to yield a semi-solid residue of 210 g. This residue was dissolved in dichloromethane (250 mL) and applied onto a column prepared from silica gel 60 (1.6 kg) and 0.5% methanol in dichloromethane) (4 L). The column was eluted with dichloromethane (7.2 L), 2), 3% methanol in dichloromethane (13 L), 5% methanol in dichloromethane (13 L), 10% methanol in dichloromethane (18 L). One 10 L fraction was taken, and then 2.5 L fractions were taken. The fractions, protected from light were sampled, flushed with argon and sealed. The fractions taken were analyzed by TLC (10% methanol in dichloromethane, UV). Fractions holding DiVA-PEG-DiVA were further analyzed by HPLC. 5 Fractions <85% pure (gave 32 g of evaporation residue) were re-purified in the same manner, using only 25% of the original amounts of silica gel and solvents. The fractions >85% pure by HPLC were combined and evaporated at reduced pressure, using a water bath of 35-40° C. The evaporation residue (120 g) was re-dissolved in dichloromethane (1.5 L) and slowly passed (approximately 1 hour) through a column prepared from ion exchanger Dowex 650C, H⁺ form (107 g). The column was then washed with dichloromethane (1 L). The combined eluate (3277.4 g) was mixed well and a sample (25 mL, 33.33 g) was evaporated, finally at room temperature and a pressure of <0.1 mBar to afford 0.83 g of a foam. From this figure the total amount of solid material was thus calculated to a yield of 80.8 g (72.5%). The remaining 3.24 kg of solution was concentrated to 423 g. 266 g of this solution was concentrated further to yield a syrup and then re-dissolved in abs. ethanol (200 mL). Evaporation at reduced pressure, using a water bath of 35-40° C., was continued to yield a final ethanol solution of 94.8 g holding 50.8 g (53.6% w/w) of N1,N19-bis((S,23E,25E,27E,29E)-16-((2E,4E,6E,8E)-3,7-dimethyl-9-(2,6,6-trimethylcyclohex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide, also known as DiVA-PEG-DiVA, also known as “DiVA”. Characterized by NMR & QTOF. ¹H NMR (400 MHz), δ_{H}: 7.07 (t, 2H), 7.01 (t, 2H), 6.87-6.91 (m, 4.0H), 6.20-6.24 (m, 10H), 6.10-6.13 (m, 8H), 5.79 (s, 2H), 5.71 (s, 2H), 4.4 (q, 2H), 3.70 (t, 6H), 3.55-3.65 (m, ˜34H), 3.59 (t, 6H), 3.4 (m, 2H), 3.25-3.33 (m, 10H), 3.16 (m, 2H), 2.44 (t, 4H), 2.33 (s, 12H), 1.97-2.01 (m, 12H), 1.96 (s, 6H), 1.7-1.9 (m, 12H), 1.69 (s, 12H), 1.5-1.65 (m, 12H), 1.35-1.5 (m, 24H), 1.01 (s, 24H). QTOF MS ESI+: m/z 2128 (M+H⁺).

### Example 22

### DOPE-VA

### Preparation of (Z)-(2R)-3-(((2-((2E,4E,6E,8E)-3,7-dimethyl-9-(2,6,6-trimethylcyclohex-1-en-1-yl)nona-2,4,6,8-tetraenamido)ethoxy)(hydroxy)phosphoryl)oxy)propane-1,2-diyl dioleate (DOPE-VA)

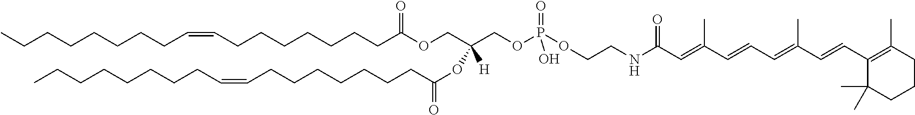 [SMILES: CCCCCCCC/C=C\CCCCCCCC(=O)OC[C@H](COP(=O)(O)OCCNC(=O)/C=C(C)/C=C/C=C(C)/C=C/C1=C(C)CCCC1(C)C)OC(=O)CCCCCCC/C=C\CCCCCCCC]

### Preparation of DOPE-VA: (Z)-(2R)-3-4(24(2E,4E,6E,8E)-3,7-dimethyl-9-(2,6,6-trimethylcyclohex-1-en-1-yl)nona-2,4,6,8-tetraenamido)ethoxy)(hydroxy)phosphoryl)oxy) pro dioleate

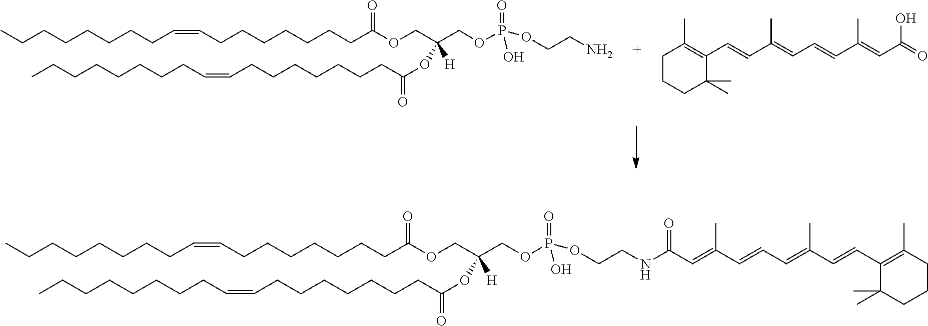 [SMILES: CCCCCCCC/C=C\CCCCCCCC(=O)OC[C@H](COP(=O)(O)OCCN)OC(=O)CCCCCCC/C=C\CCCCCCCC ; CC1=C(/C=C/C(C)=C/C=C/C(C)=C/C(=O)O)C(C)(C)CCC1 ; CCCCCCCC/C=C\CCCCCCCC(=O)OC[C@H](COP(=O)(O)OCCNC(=O)/C=C(C)/C=C/C=C(C)/C=C/C1=C(C)CCCC1(C)C)OC(=O)CCCCCCC/C=C\CCCCCCCC]

To a solution of retinoic acid (250 mg, 0.83 mmol) in diethyl ether stirring (20 mL) at −78° C., a solution of (diethylamino)sulfur trifluoride (130 μl, 0.90 mmol) in cold ether (20 mL) was added through a syringe. The reaction mixture was taken out of the cold bath and the stirring was continued at room temperature for an additional 2 hr. At the end, the solvent was removed by rotary evaporation. The residue was redissolved chloroform (50 mL) in the presence of solid Na₂CO₃(50 mg). To this solution was added 1,2-dioleoyl-sn-glycero-3-phosphoethanolamine (600 mg, 0.81 mmol) and the reaction mixture was stirred at room temperature for an additional 24 hrs. The solvent was removed by rotary evaporation. The residue was purified by silica gel chromatography with a dichloromethane/methanol gradient to yield Z)-(2R)-3-(((2-((2E,4E,6E,8E)-3,7-dimethyl-9-(2,6,6-trimethylcyclohex-1-en-1-yl)nona-2,4,6,8-tetraenamido)ethoxy)(hydroxy)phosphoryl)oxy)propane-1,2-diyl dioleate (240 mg, 28%). ¹H NMR (400 MHz, CDCl₃) δ 0.87 (t, 6H, CH₃), 1.01 (s, 6H, CH₃) 1.20-1.40 (m, 40H, CH₂), 1.40-1.60 (m, 8H, CH₂), 1.70 (s, 3H, CH₃—C═C), 1.80-2.10 (m, 811), 2.32 (m, 41-1, CH₂C(═O)), 3.50 (m, 2H), 3.92-4.18 (m, 5H), 4.35 (m, 2H), 5.20 (m, 11-1, NHC(═O)), 5.31 (m, 41-1, CH═CH), 5.80-6.90 (m, 61-1, CH═CH).

### Example 23

### DC-VA

### Preparation of (a2E,4E,6E,8E)-3,7-dimethyl-9-(2,6,6-trimethylcyclohex-1-en-1-yl)nona-2,4,6,8-tetraenoyl)azanediyl)bis(ethane-2,1-diyl)ditetradecanoate (DC-VA)

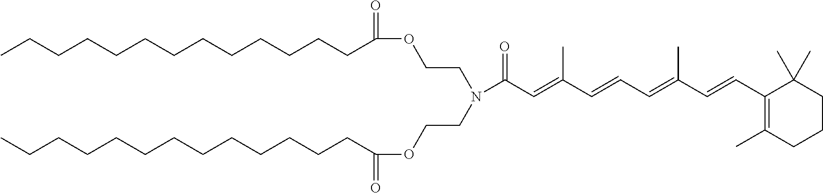 [SMILES: CCCCCCCCCCCCCC(=O)OCCN(CCOC(=O)CCCCCCCCCCCCC)C(=O)/C=C(C)/C=C/C=C(C)/C=C/C1=C(C)CCCC1(C)C]

### Preparation of DC-VA: (((2E,4E,6E,8E)-3,7-dimethyl-9-(2,6,6-trimethylcyclohex-1-en-1-yl)nona-2,4,6,8-tetraenoyl)azanediyl)bis(ethane-2,1-diyl)ditetradecanoate

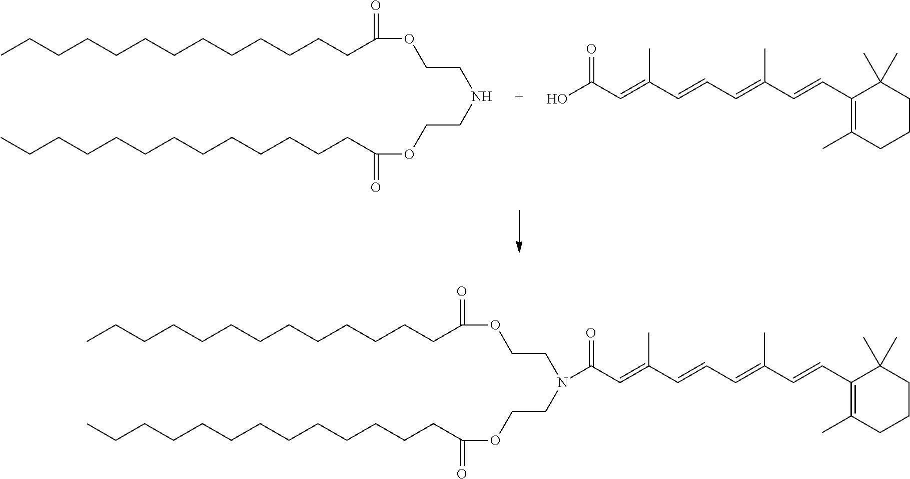 [SMILES: CCCCCCCCCCCCCC(=O)OCCNCCOC(=O)CCCCCCCCCCCCC ; CC1=C(/C=C/C(C)=C/C=C/C(C)=C/C(=O)O)C(C)(C)CCC1 ; CCCCCCCCCCCCCC(=O)OCCN(CCOC(=O)CCCCCCCCCCCCC)C(=O)/C=C(C)/C=C/C=C(C)/C=C/C1=C(C)CCCC1(C)C]

To a solution of retinoic acid (600 mg, 2.0 mmol) in diethyl ether (25 mL) stirring at −78° C., a solution of (diethylamino)sulfur trifluoride (0.3 ml, 2.1 mmol) in 5 mL of cold ether was added through a syringe. The reaction mixture was taken out of the cold bath and the stirring was continued at room temperature for an additional 1 hr. After the solvent was removed by rotary evaporation, the residue was re-dissolved in dichloromethane (20 mL) in the presence of 2 solid Na₂CO₃ (25 mg). To this solution was added the azanediylbis(ethane-2,1-diyl)ditetradecanoate (1.05 g, 2.0 mmol), and the reaction mixture was stirred at room temperature for an additional 24 hrs. The reaction mixture was diluted with dichloromethane (50 mL) and was dried over MgSO₄. After the solvent was removed by rotary evaporation, the residue was purified by silica gel chromatography with a dichloromethane/methanol gradient to yield (((2E,4E,6E,8E)-3,7-dimethyl-9-(2,6,6-trimethylcyclohex-1-en-1-yl)nona-2,4,6,8-tetraenoyl)azanediyl)bis(ethane-2,1-diyl)ditetradecanoate (800 mg, 50%). ¹H NMR (400 MHz, CDCl₃) δ 0.87 (t, 6H, CH₃), 1.02 (s, 6H, CH₃) 1.20-1.40 (m, 40H, CH₂), 1.40-1.60 (m, 8H, CH₂), 1.70 (s, 3H, CH₃—C═C), 1.97 (s, 31-1, CH₃—C═C), 2.05 (m, 2H, CH₂), 2.15 (s, 31-1, CH₃—C═C), 2.32 (m, 4H, CH₂C(═O)), 3.67 (m, 4H, NCH₂CH₂O), 4.15-4.30 (m, 4H, NCH₂CH₂O), 5.80-6.90 (m, 6H, CH═CH).

### Example 24

### DC-6-VA

### Preparation of ((64(2E,4E,6E,8E)-3,7-dimethyl-9-(2,6,6-trimethylcyclohex-1-en-1-yl)nona-2,4,6,8-tetraenamido)hexanoyl)azanediyl)bis(ethane-23-diyl)ditetradecanoate (DC-6-VA)

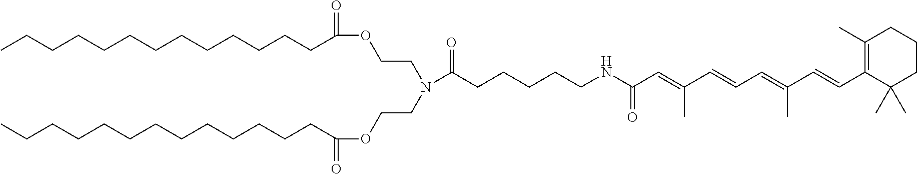 [SMILES: CCCCCCCCCCCCCC(=O)OCCN(CCOC(=O)CCCCCCCCCCCCC)C(=O)CCCCCNC(=O)/C=C(C)/C=C/C=C(C)/C=C/C1=C(C)CCCC1(C)C]

### Preparation of Intermediate 1: ((6-aminohexanoyl)azanediyl)bis(ethane-2,1-diyl)ditetradecanoate TFA salt

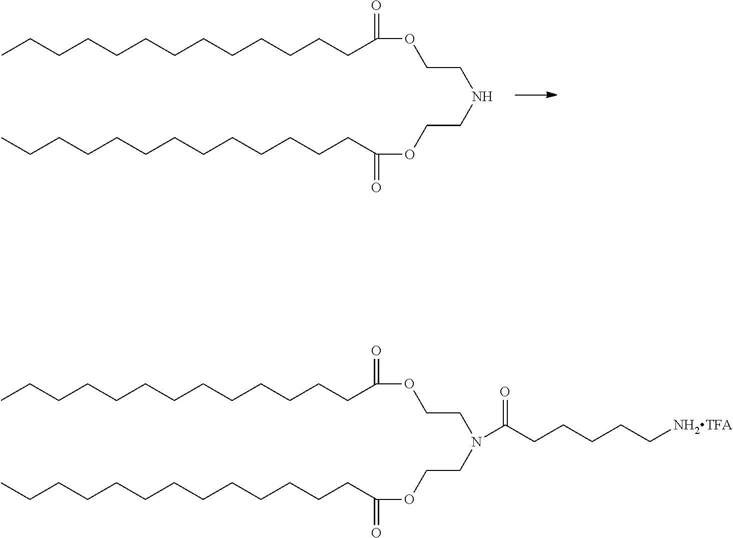

A mixture of azanediylbis(ethane-2,1-diyl)ditetradecanoate (2.5 g, 4.8 mmol), Boc-amino caproic acid (1.3 g, 5.6 mmol), N,N′-dicyclohexylcarbodiimide (1.3 g, 6.3 mmol) and N,N-diisopropylethylamine (2.6 mL, 0.015 mmol) were dissolved in pyridine (40 mL). The solution was stirred at 60° C. for overnight. The mixture was diluted with dichloromethane (50 mL) and washed with saline (3×50 mL). After being concentrated by rotary evaporation, the residue was treated with trifluoroacetic acid/dichloromethane (100 mL, 1:1). The mixture was concentrated and was re-dissolved in dichloromethane (50 mL) and washed with saline (3×50 mL). The organic layer was isolated and concentrated to yield ((6-aminohexanoyl)azanediyl)bis(ethane-2,1-diyl)ditetradecanoate TFA salt (1.5 g, 33%).

### Preparation of DC-6-VA: ((6-((2E,4E,6E,8E)-3,7-dimethyl-9-(2,6,6-trimethylcyclohex-1-en-1-yl)nona-2,4,6,8-tetraenamido)hexanoyl)azanediyl)bis(ethane-2,1-diyl)

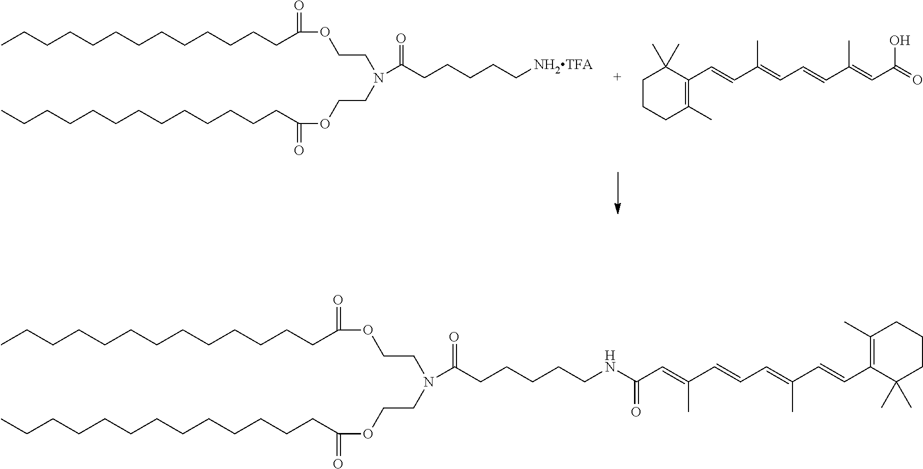

To a solution of retinoic acid (800 mg, 2.67 mmol) in diethyl ether (40 mL) stirring at −78° C., a solution of (diethylamino)sulfur trifluoride (0.4 mL, 22.80 mmol) in cold ether (7 mL) was added through a syringe. The reaction mixture was taken out of the cold bath and the stirring was continued at room temperature for an additional 1 hr. After the solvent was removed by rotary evaporation, the residue was re-dissolved in dichloromethane (25 mL) in the presence of solid Na₂CO₃ (40 mg). To this solution was added the ((6-aminohexanoyl)azanediyl)bis(ethane-2,1-diyl)ditetradecanoate TFA salt (1.5 g, 1.6 mmol) and the reaction mixture was stirred at room temperature for an additional 24 hrs. The reaction mixture was diluted with dichloromethane (50 mL) and dried over MgSO₄. After the solvent was removed by rotary evaporation, the residue was purified by column chromatography using 5% methanol/dichloromethane as eluent to yield ((6-((2E,4E,6E,8E)-3,7-dimethyl-9-(2,6,6-trimethylcyclohex-1-en-1-yl)nona-2,4,6,8-tetraenamido)hexanoyl)azanediyl)bis(ethane-2,1-diyl) (360 mg, 24%). ¹H NMR (400 MHz, CDCl₃) δ 0.87 (t, 6H, CH₃), 1.02 (s, 6H, CH₃) 1.20-1.40 (m, 42H, CH₂), 1.40-1.60 (m, 12H, CH₂), 1.70 (s, 3H, CH₃—C═C), 1.97 (s, 3H, CH₃—C═C), 2.05 (m, 2H, CH₂), 2.15 (s, 3H, CH₃—C═C), 2.32 (m, 6H, CH₂C(═O)), 3.20 (m, 2H, CH₂NHC(═O)), 3.56 (m, 4H, NCH₂CH₂O), 4.15-4.30 (m, 4H, NCH₂CH₂O), 5.10 (m, 1H), 5.80-6.90 (m, 6H, CH═CH).

### Example 25

### In Vitro Evaluation of VA-siRNA-Liposome Formulations for Knockdown Efficiency in LX-2 Cell Line and Rat Primary Hepatic Stellate Cells

LX2 cells (Dr. S. L. Friedman, Mount Sinai School of Medicine, NY) were grown in DMEM (Invitrogen) supplemented with 10% fetal bovine serum (Invitrogen) at 37° C. in the incubator with 5% CO₂. Cells were trypsinized using TryPLExpress solution (Invitrogen) for 3 min at 37° C. in the incubator. The cell concentration was determined by cell counting in hemocytometer and 3000 cells/well were seeded into the 96-well plates. The cells were grown for 24 h prior to transfection.

Rat primary hepatic stellate cells (pHSCs) were isolated from Sprague-Dawley rats according to the previously published method (Nat. Biotechnol. 2008, 26(4):431-42). pHSCs were grown in DMEM supplemented with 10% fetal bovine serum. Cells were grown up to two passages after isolation before using them for in vitro screening. Cells were seeded at the cell density of 1000 cells/well in 96-well plates and grown for 48 h before using them for transfection.

Transfection with VA-siRNA-Liposome formulations: The transfection method is the same for LX-2 and pHSC cells. The VA-siRNA-Liposome or VA-siRNA-Lipoplex formulations were mixed with growth medium at desired concentrations. 100 μl of the mixture was added to the cells in 96-well plate and cells were incubated for 30 min at 37° C. in the incubator with 5% CO₂. After 30 min, medium was replaced with fresh growth medium after. After 48 h of transfection, cells were processed using Cell-to-Ct lysis reagents (Applied Biosystems) according to the manufacturer's instructions.

Quantitatve (q) RT-PCR for measuring HSP47 mRNA expression: HSP47 and GAPDH TaqMan® assays and One-Step RT-PCR master mix were purchased from Applied Biosystems. Each PCR reaction contained the following composition: One-step RT-PCR mix 5 TaqMan® RT enzyme mix 0.25 μl, TaqMan® gene expression assay probe (HSP47) 0.25 TaqMan® gene expression assay probe (GAPDH) 0.5 μl, RNase-free water 3.25 μl, Cell lysate 0.75 Total volume of 10 GAPDH was used as endogenous control for the relative quantification of HSP47 mRNA levels. Quantitative RT-PCR was performed in ViiA™ 7 realtime PCR system (Applied Biosciences) using an in-built Relative Quantification method. All values were normalized to the average HSP47 expression of the mock transfected cells and expressed as percentage of HSP47 expression compared to mock.

The siRNA referred to in the formulation protocols are double stranded siRNA sequence with 21-mer targeting HSP47/gp46 wherein HSP47 (mouse) and gp46 (rat) are homologs—the same gene in different species:

Rat HSP47-C double stranded siRNA used for in
vitro assay (rat pHSCs):
(SEQ. ID NO. 1)
Sense (5′->3′) GGACAGGCCUCUACAACUATT
(SEQ. ID NO. 2)
Antisense (3′->5′) TTCCUGUCCGGAGAUGUUGAU.

Cationic Lipid Stock Preparation: Stock solutions of cationic lipids were prepared by combining the cationic lipid with DOPE, cholesterol, and diVA-PEG-DiVA in ethanol at concentrations of 6.0, 5.1 and 2.7 and 2.4 mg/mL respectively. If needed, solutions were warmed up to about 50° C. to facilitate the dissolution of the cationic lipids into solution.

Empty Liposome Preparation: A cationic lipid stock solution was injected into a rapidly stirring aqueous mixture of 9% sucrose at 40±1° C. through injection needle(s) at 1.5 mL/min per injection port. The cationic lipid stock solution to the aqueous solution ratio (v/v) is fixed at 35:65. Upon mixing, empty vesicles formed spontaneously. The resulting vesicles were then allowed to equilibrate at 40° C. for 10 minutes before the ethanol content was reduced to ˜12%.

Lipoplex Preparation: The empty vesicle prepared according to the above method was diluted to the final volume of 1 mM concentration of cationic lipid by 9% sucrose. To the stirring solution, 100 μL of 5% glucose in RNase-free water was added for every mL of the diluted empty vesicle (“EV”) and mixed thoroughly. 150 μL of 10 mg/mL siRNA solution in RNase-free water was then added at once and mixed thoroughly. The mixture was then diluted with 5% glucose solution with 1.750 mL for every mL of the EV used. The mixture was stirred at about 200 rpm at room temperature for 10 minutes. Using a semi-permeable membrane with ˜100000 MWCO in a cross-flow ultrafiltration system using appropriately chosen peristaltic pump (e.g. Midgee Hoop, UFP-100-H24LA), the mixture was concentrated to about ⅓ of the original volume (or desired volume) and then diafiltered against 5 times of the sample volume using an aqueous solution containing 3% sucrose and 2.9% glucose. The product was then filtered through a combined filter of 0.8/0.2 micron pore size under aseptic conditions before use.

Formation of non-diVA siRNA containing liposomes: Cationic lipid, DOPE, cholesterol, and PEG conjugated lipids (e.g., Peg-Lipid) were solubilized in absolute ethanol (200 proof) at a molar ratio of 50:10:38:2. The siRNA was solubilized in 50 mM citrate buffer, and the temperature was adjusted to 35-40° C. The ethanol/lipid mixture was then added to the siRNA-containing buffer while stirring to spontaneously form siRNA loaded liposomes. Lipids were combined with siRNA to reach a final total lipid to siRNA ratio of 15:1 (wt:wt) The range can be 5:1 to 15:1, preferably 7:1 to 15:1. The siRNA loaded liposomes were diafiltered against 10× volumes of PBS (pH 7.2) to remove ethanol and exchange the buffer. Final product was filtered through 0.22 μm, sterilizing grade, PES filter for bioburden reduction. This process yielded liposomes with a mean particle diameter of 50-100 nm, PDI<0.2, >85% entrapment efficiency.

Formation of siRNA containing liposomes co-solubilized with diVA: siRNA-diVA-Liposome formulations were prepared using the method described above. diVA-PEG-diVA was co-solubilized in absolute ethanol with the other lipids (cationic lipid, DOPE, cholesterol, and PEG-conjugated lipids at a ratio of 50:10:38:2) prior to addition to the siRNA containing buffer. Molar content of diVA-PEG-diVA ranged from 0.1 to 5 molar ratio. This process yielded liposomes with a mean particle diameter of 50-100 nm, PDI<0.2, >85% entrapment efficiency.

Formation of siRNA containing liposomes with cationic lipids: siRNA-diVA-Liposome formulations and siRNA-Liposome formulations were prepared using the method described above. Cationic lipid can be, for example, DODC, HEDC, HEDODC, DC-6-14, or any combination of these cationic lipids.

Formation of siRNA containing liposomes decorated with diVA: siRNA-Liposome formulations were prepared using the method described above and diluted to a siRNA concentration of 0.5 mg/mL in PBS. Cationic lipid can be DODC, HEDC, HEDODC, DC-6-14, or any combination of these cationic lipids. diVA-PEG-diVA was dissolved in absolute ethanol (200 proof) to a final concentration ranging from 10 to 50 mg/mL. An appropriate amount of ethanol solution was added to the siRNA-Liposome solution to yield a final molar percentage between 2 to 10 mol %. Solution was plunged up and down repeatedly with a pipette to mix. diVA-PEG-diVA concentration and ethanol addition volume were adjusted to keep the addition volume >1.0 μL and the final ethanol concentration <3% (vol/vol). Decorated liposomes were then gently shaken at ambient temperature for 1 hr on an orbital shaker prior to in vitro or in vivo evaluation.

### Results

FIG. 24 shows that addition of the VA-conjugate to liposomes via decoration improved the knockdown efficacy of siRNA, enhancing siRNA activity. Peg-Lipid. The dose for all samples was 867 nM siRNA HSP47-C. The results showed that in every instance where a VA-conjugate was added to liposomes, siRNA activity was enhanced compared to liposomes without a retinoid and compared to liposomes decorated with free (non-conjugated) retinol. RNAiMAX was a commercial transfection reagent.

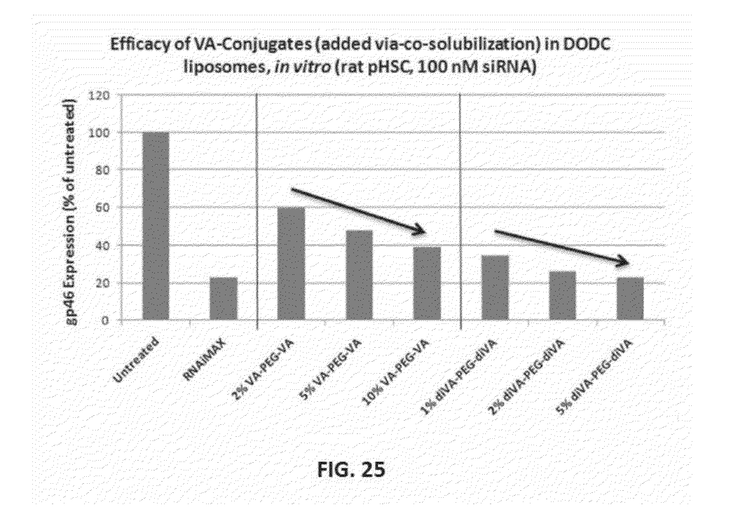

FIG. 25 shows that addition of VA-conjugates to liposomes via co-solubilization improves knockdown efficacy of siRNA. These were DODC containing liposomes with VA-conjugates added via co-solubilization. The formulation is 50:10:38:2:X, where X=1 to 10 (DODC:DOPE:cholesterol:PEG-Lipid:VA-conjugate, mole ratio). The concentration in every instance was 100 nM siRNA HSP47-C. The results show that addition of VA-conjugates to liposomes via cosolubilization enhances siRNA activity.

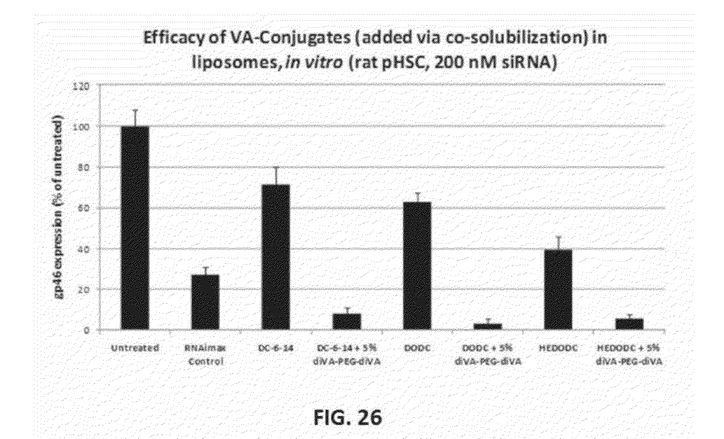

FIG. 26 shows that addition of VA-conjugate to liposomes via co-solubilization dramatically improves the knockdown efficacy of siRNA. Results include three different liposomes, DC-6-14, DODC, HEDODC with VA-conjugates added via co-solubilization. The formulation is the same for all, 50:10:38:2, cationic lipid:DOPE:cholesterol:Peg-Lipid, with only the cationic lipid varying. The concentration of siRNA is 200 nM siRNA HSP47-C is the same for all. The results show that VA-conjugate addition to liposomes having different cationic lipids significantly enhanced siRNA activity, when prepared by co-solubilization.

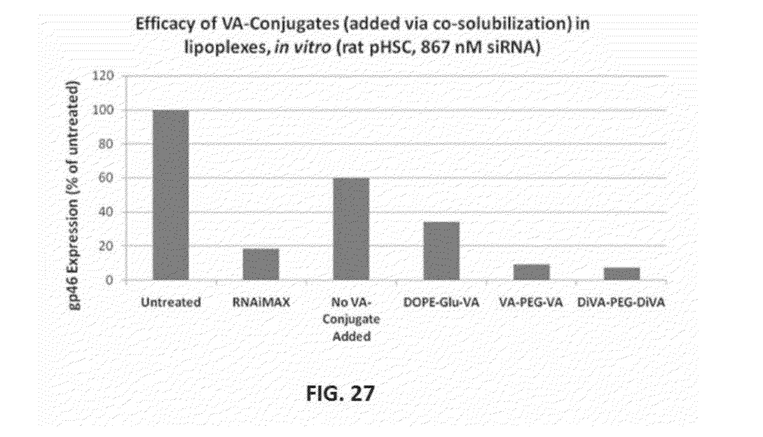

FIG. 27 shows that addition of VA-conjugates to lipoplexes having DC-6-14 cationic lipid via co-solubilization, and siRNA coating the exterior of the liposome enhances siRNA activity. The formulation is a 40% lipoplex formulation, 40:30:30, DC-6-14:DOPE:cholesterol. The concentration for all samples is 867 nM siRNA HSP47-C. The results show that VA-conjugate addition to lipoplexes via co-solubilization enhance siRNA activity.

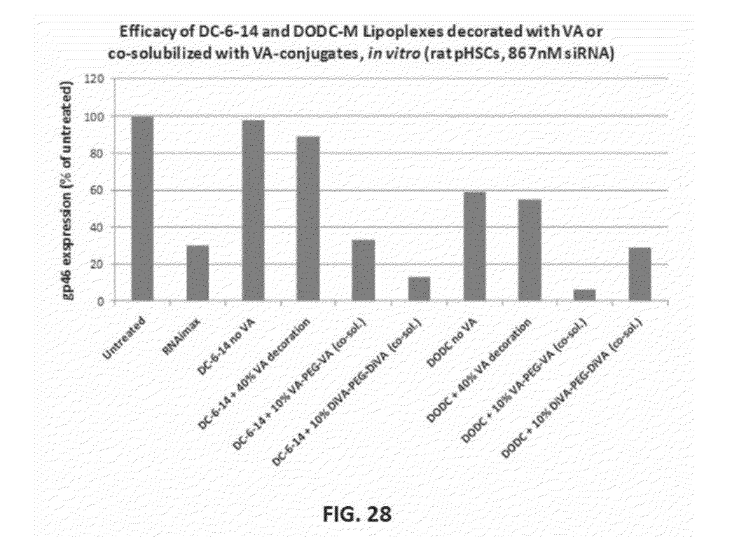

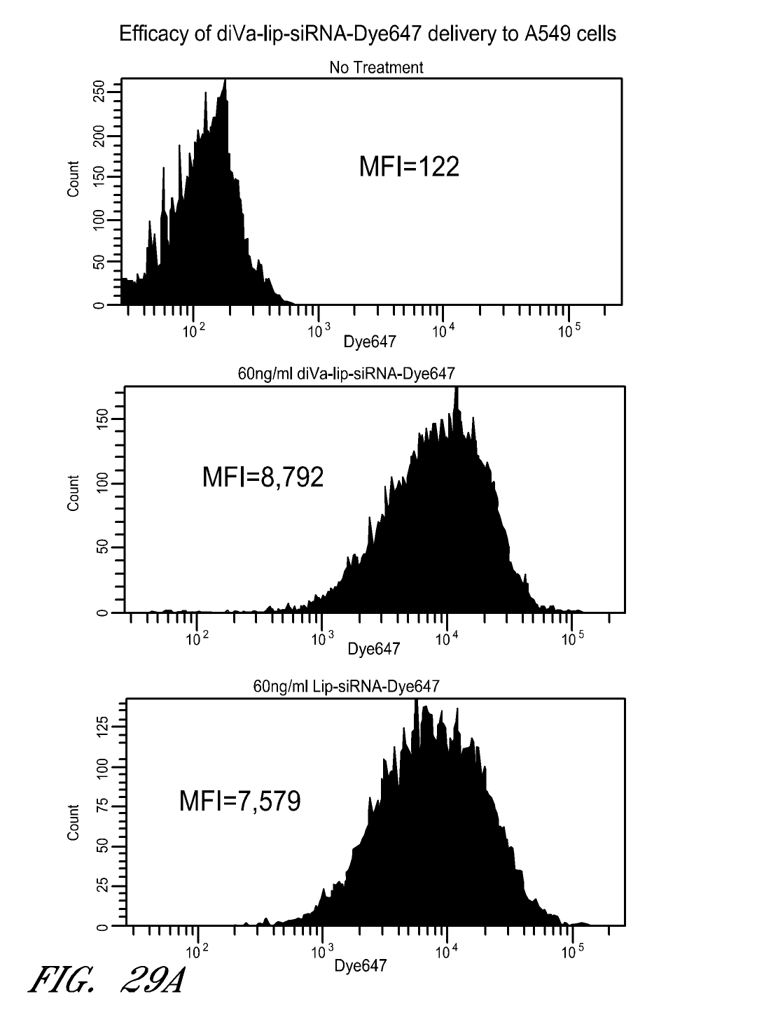

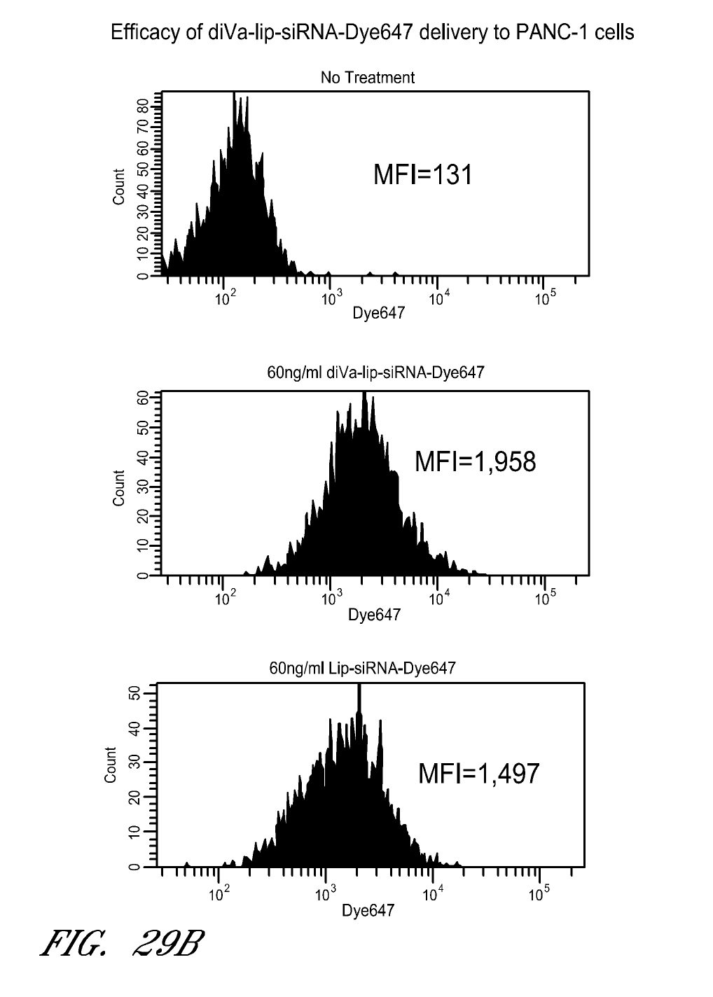

FIG. 28 shows that addition of VA-conjugate to lipoplexes formed via co-solubilization compared to lipoplexes with VA-conjugate added via decoration. These results are from DC-6-14 and DODC lipoplexes. The formulation consists of 40:30:30, DC-6-14:DOPE:cholesterol. The concentration in each sample is 867 nM siRNA HSP47-C. VA-conjugate addition via co-solubilization significantly improves knockdown efficacy in vitro, relative to VA-conjugates added by decoration.

### Example 26

### Synthesis of satDiVA

### Preparation of N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide (satDIVA).

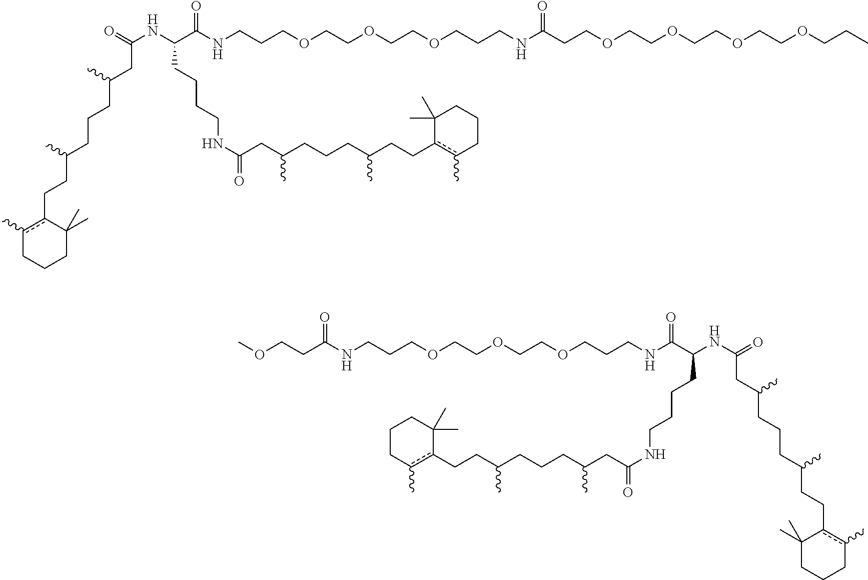

### Preparation of Intermediate 1: 3,7-dimethyl-9-(2,6,6-trimethylcyclohex-1-en-1-yl)nonanoic acid

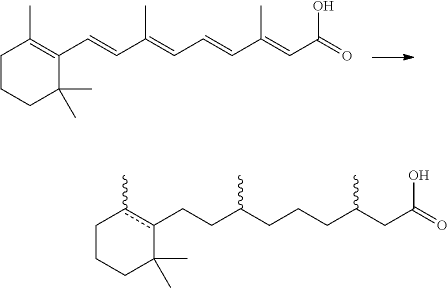

All-trans retinoic acid (2000 mg, 6.66 mmol) was dissolved in hexanes/IPA (3:1, 40 mL) with the aid of sonication. Material was placed in a Parr-shaker bottle and flushed with inert gas. 10% Pd/C (200 mg) was added and the vessel was once again flushed with inert gas. Material was placed on the Parr-Shaker overnight with >70 psi Hydrogen gas. The reaction mixture was then filtered through a pad of celite and concentrated to yield 3,7-dimethyl-9-(2,6,6-trimethylcyclohex-1-en-1-yl)nonanoic acid (2 g).

### Preparation of satDIVA: N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide

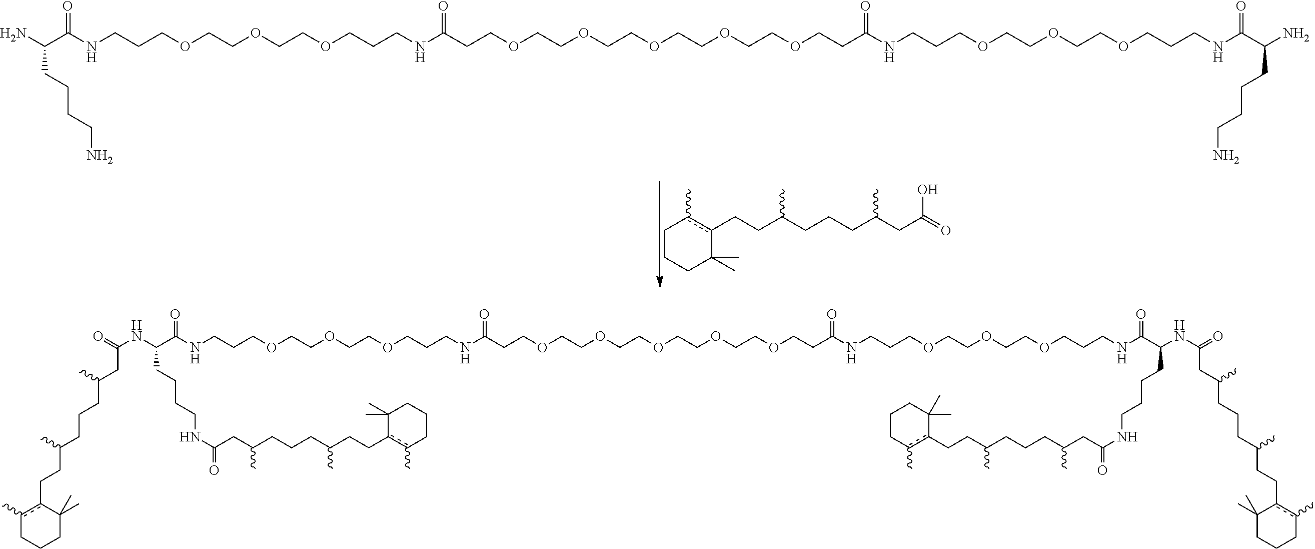

N1,N19-bis((16S)-16-(3,7-dimethyl-9-(2,6,6-trimethylcyclohex-1-en-1-yl)nonanamido)-24,28-dimethyl-15,22-dioxo-30-(2,6,6-trimethylcyclohex-1-en-1-yl)-4,7,10-trioxa-14,21-diazatriacontyl)-4,7,10,13,16-pentaoxanonadecane-1,19-diamide, also known as satDIVA, was prepared in similar fashion as diva-PEG-diVA from previously described N1,N19-bis((S)-16,20-diamino-15-oxo-4,7,10-trioxa-14-azaicosyl)-4,7,10,13,16-pentaoxanonadecane-1,19-diamide with the substitution of 3,7-dimethyl-9-(2,6,6-trimethylcyclohex-1-en-1-yl)nonanoic acid for all-trans retinoic acid. QTOF MS ESI+: m/z 2161, 2163, 2165 & 2167 (M+H+).

### Example 27

### Synthesis of simDiVA

### Preparation of N1,N19-bis((S)-15,22-dioxo-30-(2,6,6-trimethylcyclohex-1-en-1-yl)-16-(9-(2,6,6-trimethylcyclohex-1-en-1-yl)nonanamido)-4,7,10-trioxa-14,21-diazatriacontyl)-4,7,10,13,16-pentaoxanonadecane-1,19-diamide (simDiVA)

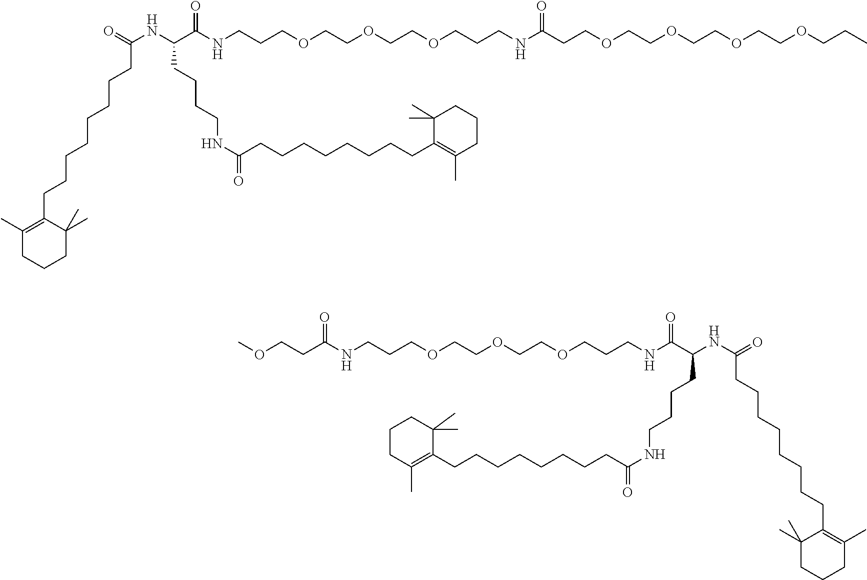

### Preparation of Intermediate 1: 2,6,6-trimethylcyclohex-1-en-1-yl trifluoromethanesulfonate

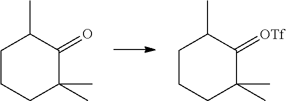

To a solution of 2,2,6-trimethylcyclohexanone in dry THF at −78° C. under nitrogen was added dropwise a 2 M lithium diisopropylamide solution. The mixture was stirred at −78° C. for 3 h. A solution of N-phenyl-bis(trifluoromethanesulfonimide) in THF was then added dropwise (at −78° C.). The reaction flask was packed in dry-ice and stirred overnight. The stirring was continued at room temperature for 3 h under which time all material had dissolved. The reaction mixture was concentrated and the residue was added slowly to hexane (350 mL) under vigorous stirring. The solid material was removed by filtration and washed with hexane (2×50 mL). The filtrate was concentrated and more hexane (150 mL) was added. The solid material was removed by filtration and the filtrate was concentrated. The precipitation was repeated one more time after which the residue was purified by flash chromatography (silica, hexane) to give 2,6,6-trimethylcyclohex-1-en-1-yl trifluoromethanesulfonate as a colorless oil (23.2 g, 60% yield).

### Preparation of Intermediate 2: ethyl 9-(bromozincio)nonanoate

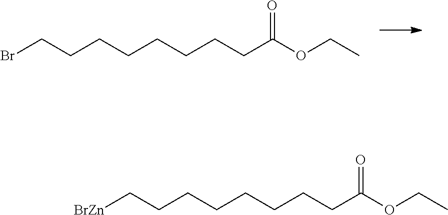

In a dry reaction tube under nitrogen were charged zinc dust (3.70 g, 56.6 mmol), iodine (479 mg, 1.89 mmol) and dry DMA (20 mL). The mixture was stirred at room temperature until the color of iodine disappeared. Ethyl 9-bromononanoate was added, and the mixture was stirred at 80° C. for 4 hours and then at room temperature overnight. (Completion of the zinc insertion reaction was checked by GCMS analysis of the hydrolyzed reaction mixture.) The reaction mixture was used without further treatment in the subsequent step. GCMS m/z 186 [M]+(ethyl nonanoate).

### Preparation of Intermediate 3: ethyl 9-(2,6,6-trimethylcyclohex-1-en-1-yl)nonanoate

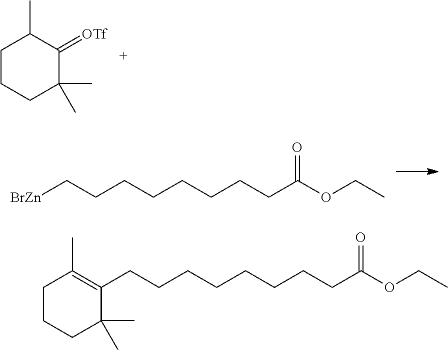

To freshly prepared ethyl 9-(bromozincio)nonanoate (37.7 mmol) in dimethylacetamide under nitrogen in a reaction tube was added 2,6,6-trimethylcyclohex-1-en-1-yl trifluoromethanesulfonate (10.8 g, 39.6 mmol) followed by tetrakis(triphenylphosphine)palladium(0) (872 mg, 0.754 mmol). The tube was sealed and the mixture was stirred at 95° C. for 2 h. The reaction mixture was allowed to cool and was then poured into diethyl ether (100 mL). The upper layer was decanted and the lower layer was washed twice with diethyl ether (2×25 mL). The combined ether layers were washed with sat NH₄Cl and brine, dried (MgSO₄) and concentrated to give crude material (˜12 g). The material was purified by flash chromatography (silica, 0 to 1.5% EtOAc in hexane). The obtained oil was stirred under vacuum for 8 h in order to remove most of the side-product, ethyl nonanoate, and was then purified by a second flash chromatography (silica, 0 to 15% toluene in hexane). The fractions were analyzed by LCMS and GCMS. The purest fractions were collected and concentrated at a temperature below 25° C. to give ethyl 942,6,6-trimethylcyclohex-1-en-1-yl)nonanoate as a colorless oil (6.16 g, 53% yield over two steps). LCMS ESI+m/z 309 [M+H]+; GCMS m/z 308 [M]+.

### Preparation of Intermediate 4: 9-(2,6,6-trimethylcyclohex-1-en-1-yl)nonanoic acid

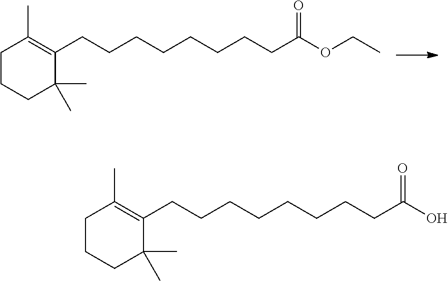 [SMILES: CCOC(=O)CCCCCCCCC1=C(C)CCCC1(C)C ; CC1=C(CCCCCCCCC(=O)O)C(C)(C)CCC1]

To ethyl 9-(2,6,6-trimethylcyclohex-1-en-1-yl)nonanoate (13.2 g, 42.9 mmol) in ethanol (80 mL) was added 4 M KOH (43 mL). The mixture was stirred at room temperature for 1.5 h. Water (350 mL) was added and the solution was washed with tert-butyl methyl ether (2×100 mL). The SimVA, aqueous phase was cooled, acidified with 4 M HCl (˜45 mL) and extracted with pentane (3×100 mL). The combined pentane extracts were washed with water (200 mL), dried (MgSO4), filtered, concentrated and dried under high vacuum. The material was redissolved in pentane (100 mL), concentrated and dried under high vacuum one more time to give 9-(2,6,6-trimethylcyclohex-1-en-1-yl)nonanoic acid as a colorless oil (11.1 g, 92% yield). MS ESI-m/z 279 [M−H]-.

### Preparation of simdiVA: N1,N19-bis((S)-15,22-dioxo-30-(2,6,6-trimethylcyclohex-1-en-1-yl)-16-(9-(2,6,6-trimethylcyclohex-1-en-1-yl)nonanamido)-4,7,10-trioxa-14,21-diazatriacontyl)-4,7,10,13,16-pentaoxanonadecane-1,19-diamide.

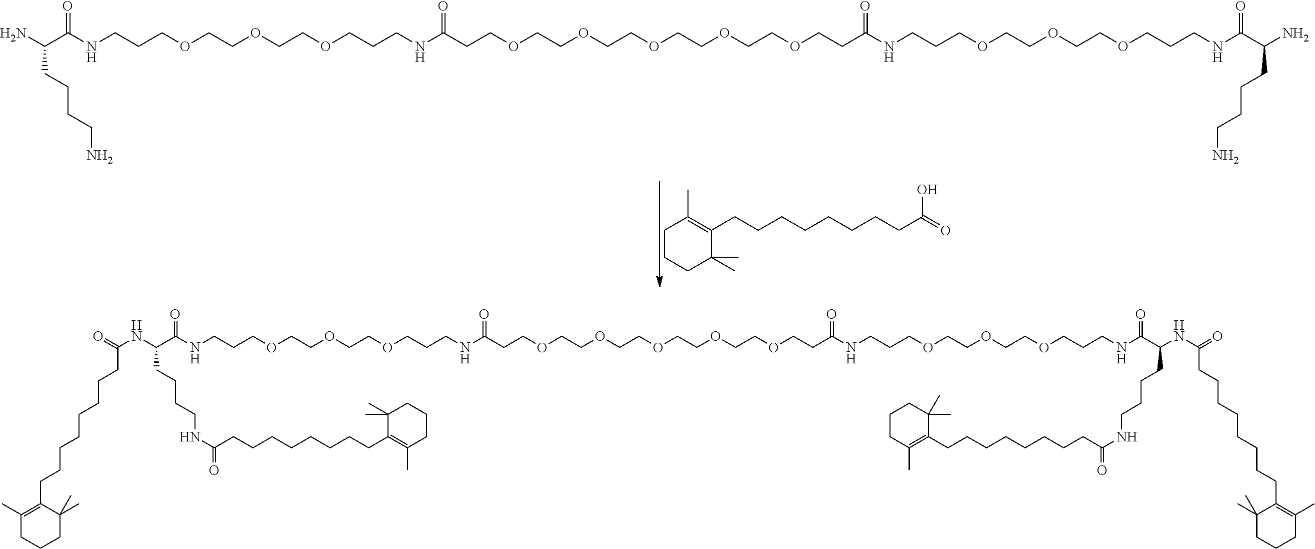

simDIVA was prepared in similar fashion as diVA from previously described N1,N19-bis((S)-16,20-diamino-15-oxo-4,7,10-trioxa-14-azaicosyl)-4,7,10,13,16-pentaoxanonadecane-1,19-diamide with the substitution of 9-(2,6,6-trimethylcyclohex-1-en-1-yl)nonanoic acid for all-trans retinoic acid. QTOF MS ESI+: m/z 2050 (M+H+)

### Example 28

### Synthesis of DiVA-PEG18

### Preparation of (2E,2′E,2″E,4E,4′E,4″E,6E,6′E,6″E,8E,8′E,8″E)-N,N′,N″-((5R,69R,76E,78E,80E,82E)-77,81-dimethyl-6,68,75-trioxo-83-(2,6,6-trimethylcyclohex-1-en-1-yl)-10,13,16,19,22,25,28,31,34,37,40,43,46,49,52,55,58,61,64-nonadecaoxa-7,67,74-triazatrioctaconta-76,78,80,82-tetraene-1,5,69-triyl)tris(3,7-dimethyl-9-(2,6,6-trimethylcyclohex-1-en-1-yl)nona-2,4,6,8-tetraenamide) (DIVA-PEG18).

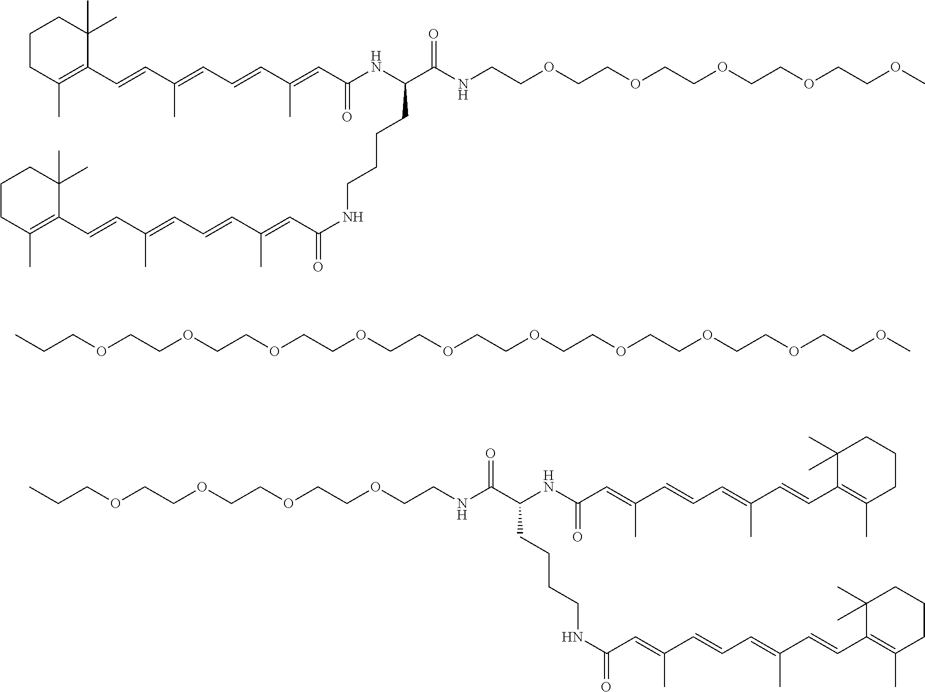

(2E,2′E,2″E,4E,4′E,4″E,6E,6′E,6″E,8E,8′E,8″E)-N,N′,N″-((5R,69R,76E,78E,80E,82E)-77,81-dimethyl-6,68,75-trioxo-83-(2,6,6-trimethylcyclohex-1-en-1-yl)-10,13,16,19,22,25,28,31,34,37,40,43,46,49,52, 55,58,61,64-nonadecaoxa-7,67,74-triazatrioctaconta-76,78,80,82-tetraene-1,5,69-triyl)tris(3,7-dimethyl-9-(2,6,6-trimethylcyclohex-1-en-1-yl)nona-2,4,6,8-tetraenamide), also known as DIVA-PEG18 was prepared in similar fashion as diVA with the substitution of PEG₁₈ diamine for diamido-dPEG₁₁-diamine. LCMS ESI+: m/z 2305 (M+Na).

### Example 29

### Synthesis of TriVA

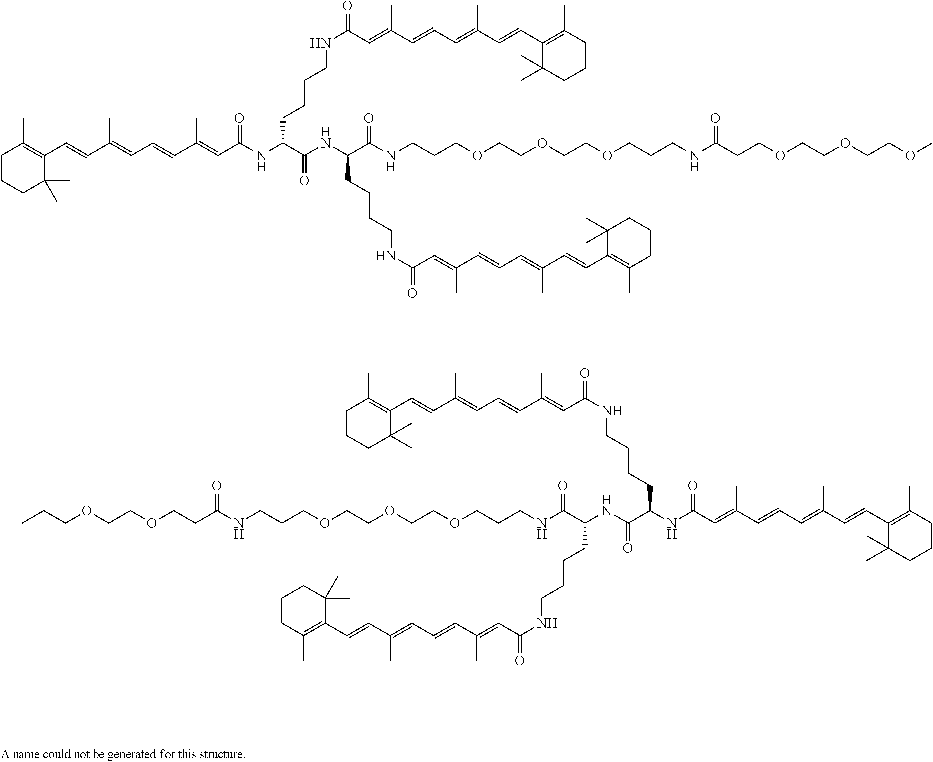

Preparation of Intermediate 1: (S)-methyl 6-(((benzyloxy)carbonyl)amino)-2-((S)-2,6-bis(((benzyloxy) carbonyl)amino)hexanamido) hexanoate

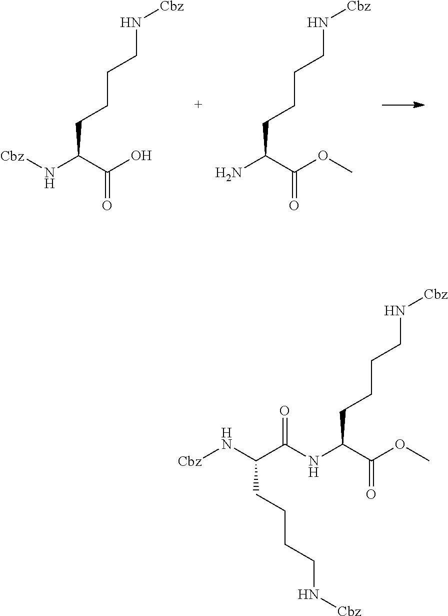 [SMILES: O=C(NCCCC[C@H](NC(=O)OCc1ccccc1)C(=O)O)OCc1ccccc1 ; COC(=O)[C@@H](N)CCCCNC(=O)OCc1ccccc1 ; COC(=O)[C@H](CCCCNC(=O)OCc1ccccc1)NC(=O)[C@H](CCCCNC(=O)OCc1ccccc1)NC(=O)OCc1ccccc1]

A flask was purged with inert gas and H-Lys(Z)-OMe HCl salt (4 g, 12.1 mmol), HOBt hydrate (1.84 g, 13.6 mmol), Z-Lys(Z)-OH (5.64 g, 13.6 mmol) are suspended in dichloromethane (50 mL). NMM (1.5 mL, 13.6 mmol) was added to the suspension and the solution became clear. A suspension EDC HCl salt (4.01 g, 20.9 mmol) and NMM (2.0 mL, 18.2 mmol) in dichloromethane (50 mL) was added over a period of 10 minutes. The reaction was stirred overnight at room temperature, then washed with 1M HCl (100 mL), H₂O (100 mL), saturated bicarbonate solution (100 mL) and saturated brine solution (100 mL). All aqueous washes were back extracted with dichloromethane (50 mL). Dried organics with Na₂SO₄, filtered and concentrated. Material was purified by silica gel chromatography with a dichloromethane/methanol gradient to yield (S)-methyl 6-(((benzyloxy)carbonyl)amino)-2-((S)-2,6-bis(((benzyloxy)carbonyl)amino)hexanamido) hexanoate (6.91 g).

### Preparation of Intermediate 2: (S)-6-(((benzyloxy)carbonyl)amino)-2-(S)-2,6-bis(((benzyloxy)carbonyl)amino)hexanamido)hexanoic acid

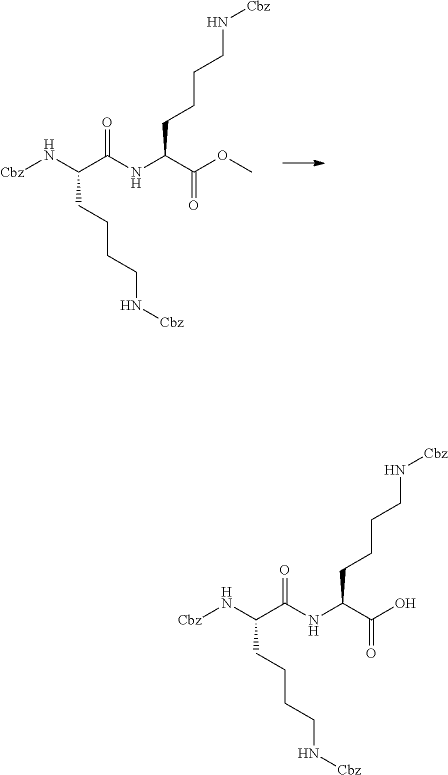 [SMILES: COC(=O)[C@H](CCCCNC(=O)OCc1ccccc1)NC(=O)[C@H](CCCCNC(=O)OCc1ccccc1)NC(=O)OCc1ccccc1 ; O=C(NCCCC[C@H](NC(=O)[C@H](CCCCNC(=O)OCc1ccccc1)NC(=O)OCc1ccccc1)C(=O)O)OCc1ccccc1]

6-(((benzyloxy)carbonyl)amino)-2-((S)-2,6-bis(((benzyloxy)carbonyl)amino)hexanamido) hexanoate (6.91 g, 10 mmol) was dissolved with methanol (50 mL). Added KOH (2.24 g, 40 mmol) and allowed mixture to stir at 35° C. After 2 hours, quenched reaction by adding H₂O (200 mL) and washed mixture with diethyl ether (50 mL). Afterwards, adjusted the pH to ˜2 with 1M HCl acid. Extracted product with dichloromethane (3×100 mL), dried with Na₂SO₄, filtered and concentrated to yield (S)-6-(((benzyloxy)carbonyl)amino)-2-((S)-2,6-bis(((benzyloxy)carbonyl)amino)hexanamido)hexanoic acid (4 g).

### Preparation of Intermediate 3: (Cbz)₆-protected N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide

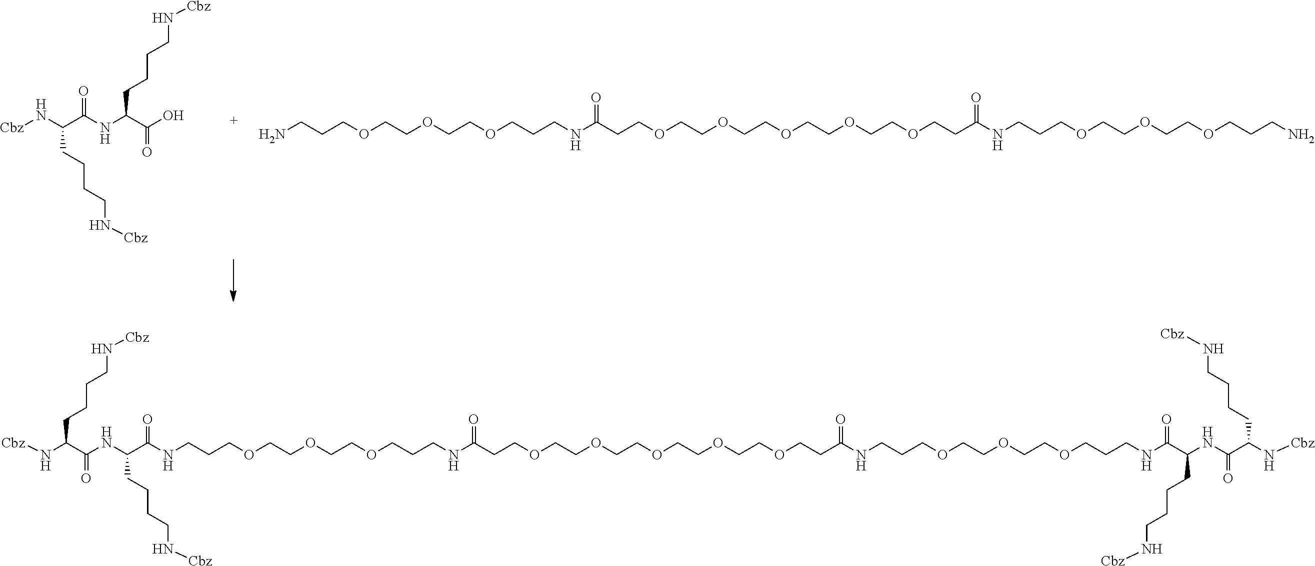 [SMILES: O=C(NCCCC[C@H](NC(=O)[C@H](CCCCNC(=O)OCc1ccccc1)NC(=O)OCc1ccccc1)C(=O)O)OCc1ccccc1 ; NCCCOCCOCCOCCCNC(=O)CCOCCOCCOCCOCCOCCC(=O)NCCCOCCOCCOCCCN ; O=C(CCOCCOCCOCCOCCOCCC(=O)NCCCOCCOCCOCCCNC(=O)[C@H](CCCCNC(=O)OCc1ccccc1)NC(=O)[C@H](CCCCNC(=O)OCc1ccccc1)NC(=O)OCc1ccccc1)NCCCOCCOCCOCCCNC(=O)[C@H](CCCCNC(=O)OCc1ccccc1)NC(=O)[C@H](CCCCNC(=O)OCc1ccccc1)NC(=O)OCc1ccccc1]

A round bottom flask was purged with inert gas and diamido-dPEG₁₁-diamine (1 g, 1.35 mmol), (S)-6-(((benzyloxy)carbonyl)-amino)-2-((S)-2,6-bis(((benzyloxy)carbonyl)amino)hexanamido)hexanoic acid (2.05 g, 3.03 mmol), HOBt hydrate (409 mg, 3.03 mmol) are suspended in dichloromethane (25 mL). NMM (333 μL, 3.03 mmol) was added to the suspension and the solution became clear. A suspension EDC HCl salt (893 mg, 4.66 mmol) and NMM (445 μL, 4.05 mmol) in dichloromethane (25 mL) was added over a period of 10 minutes. The reaction was allowed to stir overnight at room temperature, then washed with 1M HCl (100 mL), H₂O (100 mL), saturated bicarbonate solution (100 mL) and saturated brine solution (100 mL). All aqueous washes were back extracted with dichloromethane (50 mL). Dried organics with Na₂SO₄, filtered and concentrated. Material was purified by silica gel chromatography with a dichloromethane/methanol gradient to yield (Cbz)₆-protected N1,N19-bis((16 S,19S)-19,23-diamino-16-(4-aminobutyl)-15,18-dioxo-4,7,10-trioxa-14,17-diazatricosyl)-4,7,10,13,16-pentaoxanonadecane-1,19-diamide (480 mg).

### Preparation of Intermediate 4: N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide

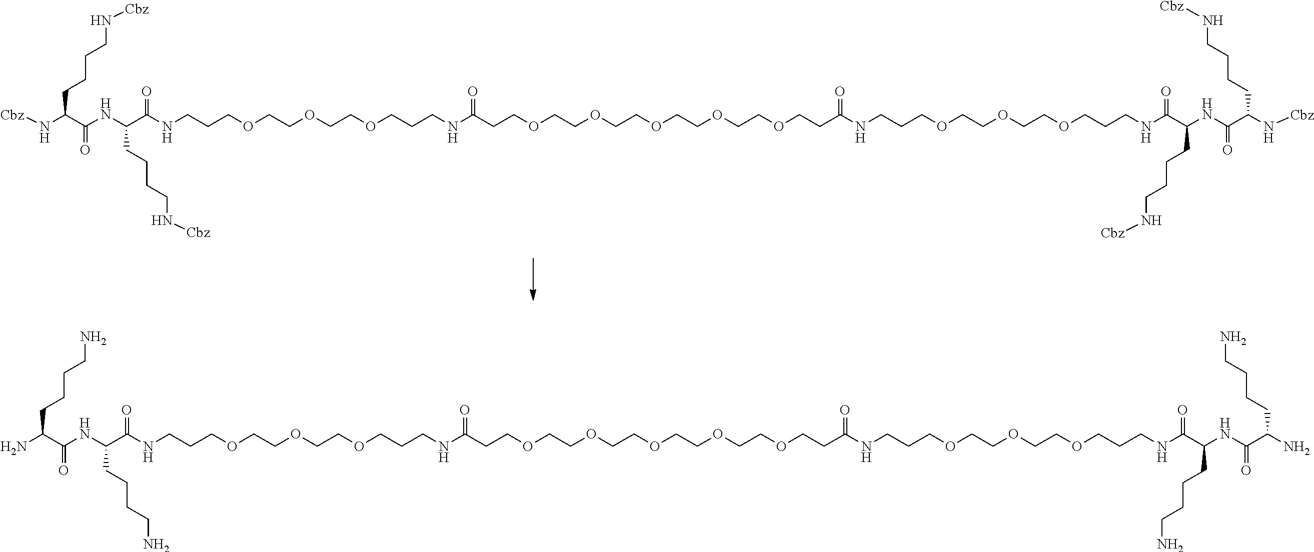 [SMILES: O=C(CCOCCOCCOCCOCCOCCC(=O)NCCCOCCOCCOCCCNC(=O)[C@H](CCCCNC(=O)OCc1ccccc1)NC(=O)[C@H](CCCCNC(=O)OCc1ccccc1)NC(=O)OCc1ccccc1)NCCCOCCOCCOCCCNC(=O)[C@H](CCCCNC(=O)OCc1ccccc1)NC(=O)[C@H](CCCCNC(=O)OCc1ccccc1)NC(=O)OCc1ccccc1 ; NCCCC[C@H](NC(=O)[C@@H](N)CCCCN)C(=O)NCCCOCCOCCOCCCNC(=O)CCOCCOCCOCCOCCOCCC(=O)NCCCOCCOCCOCCCNC(=O)[C@H](CCCCN)NC(=O)[C@@H](N)CCCCN]

(Cbz)₆-protected N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide was dissolved in methanol (30 mL) in a round bottom flask and flushed with an inert gas. 10% Pd/C (135 mg) was added and the flask was once again flushed with inert gas and then all air was removed via vacuum pump. An 8″ H₂ balloon was added and the reaction was allowed to stir at room temperature. After 2 hours, the Pd/C was removed by filtering through a pad of celite washing with methanol, and concentrated to yield N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide (823 mg).

### Preparation of TriVA

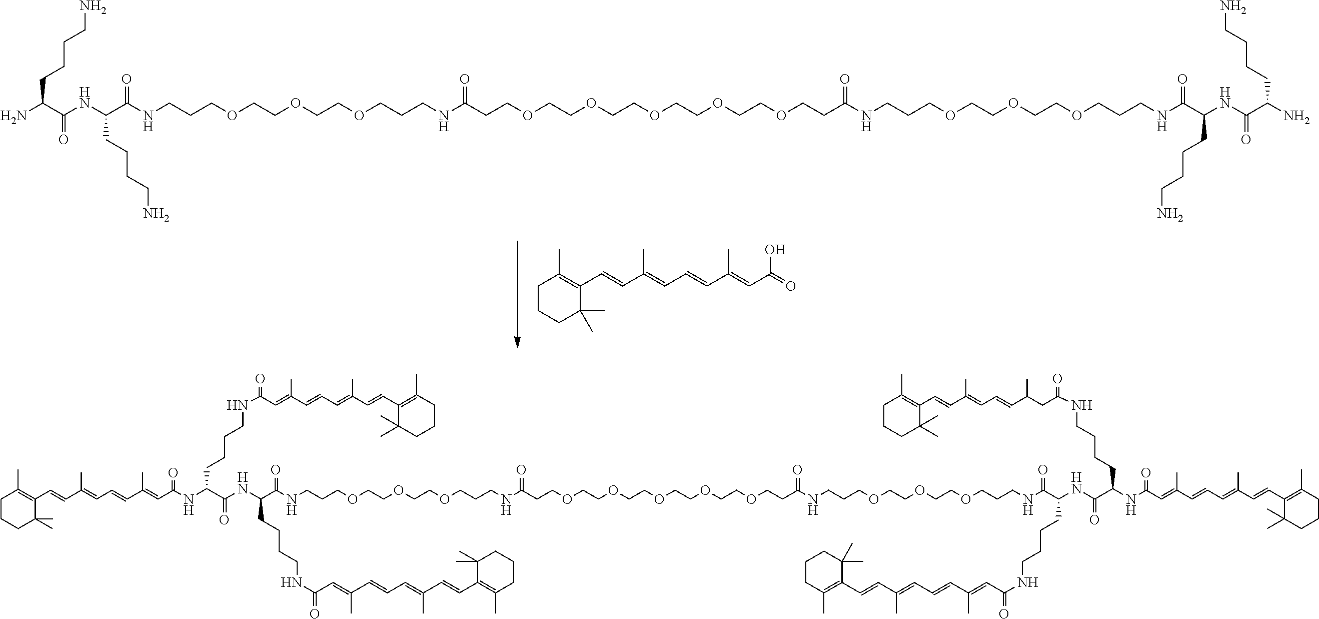

N1,N19-bis((16S,19S)-19,23-diamino-16-(4-aminobutyl)-15,18-dioxo-4,7,10-trioxa-14,17-diazatricosyl)-4,7,10,13,16-pentaoxanonadecane-1,19-diamide was stirred in dichloromethane and DMAP and retinoic acid was added. NMM was added and the solution was stirred in an aluminum foil covered round bottom flask flushed with inert gas at room temperature. A suspension of EDC HCl salt & NMM in dichloromethane (20 mL) was slowly added to reaction over a period of 10 minutes. Reaction was allowed to stir overnight at room temperature. Next day, diluted with dichloromethane to 100 mL. Washed with H₂O (100 mL), saturated bicarbonate solution (100 mL) and saturated brine solution (100 mL). All aqueous washes were back extracted with dichloromethane (50 mL). Dried organics with Na₂SO₄, filtered and concentrated. Material was purified by basic alumina chromatography eluating with dichloromethane/ethanol gradient to yield TriVA (780 mg). LCMS ESI+: m/z 2972 (M+Na).

### Example 30

### Synthesis of 4TTNPB

Preparation of N1,N19-bis((R)-1,8-dioxo-7-(4-((E)-2-(5,5,8,8-tetramethyl-5,6,7,8-tetrahydro-naphthalen-2-yl)prop-1-en-1-yl)benzamido)-1-(4-((E)-2-(5,5,8,8-tetramethyl-5,6,7,8-tetrahydronaphthalen-2-yl)prop-1-en-1-yl)phenyl)-13,16,19-trioxa-2,9-diazadocosan-22-yl)-4,7,10,13,16-pentaoxanonadecane-1,19-diamide (4TTNPB).

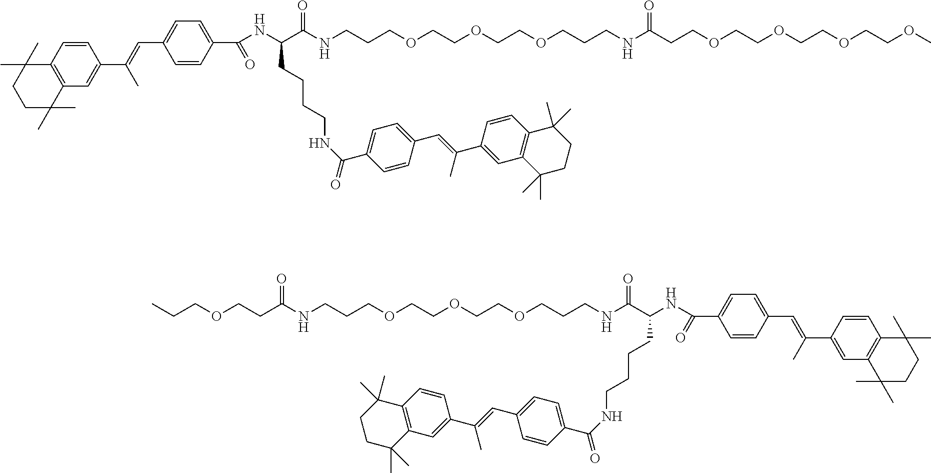

N1,N19-bis((R)-1,8-dioxo-7-(4-((E)-2-(5,5,8,8-tetramethyl-5,6,7,8-tetrahydro-naphthalen-2-yl)prop-1-en-1-yl)benzamido)-1-(4-((E)-2-(5,5,8,8-tetramethyl-5,6,7,8-tetrahydronaphthalen-2-yl)prop-1-en-1-yl)phenyl)-13,16,19-trioxa-2,9-diazadocosan-22-yl)-4,7,10,13,16-pentaoxanonadecane-1,19-diamide, also known as 4TTNPB, was prepared in similar fashion as N1,N19-bis((S,23E,25E,27E,29E)-164(2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide, also known as diVA, from N1,N19-bis((S)-16,20-diamino-15-oxo-4,7,10-trioxa-14-azaicosyl)-4,7,10,13,16-pentaoxanonadecane-1,19-diamide with the substitution of TTNPB for all-trans retinoic acid. LCMS ESI+: m/z 2343 (M+Na).

### Example 31

### Synthesis of 4Myr

Preparation of N1,N19-bis((R)-15,22-dioxo-16-tetradecanamido-4,7,10-trioxa-14,21-diazapenta-triacontyl)-4,7,10,13,16-pentaoxanonadecane-1,19-diamide (4Myr).

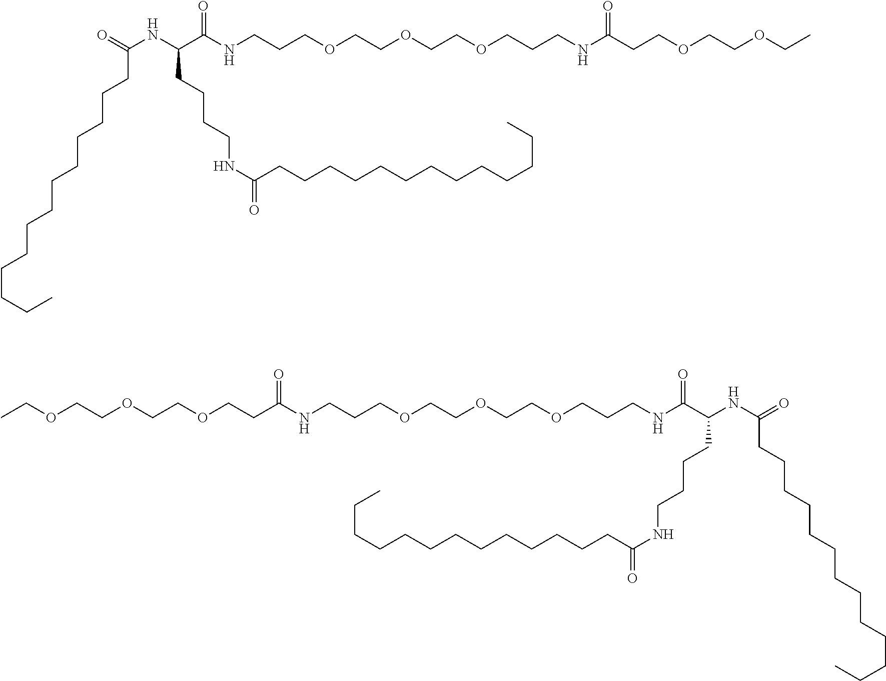

### Preparation of 4Myr: N1,N19-bis((R)-15,22-dioxo-16-tetradecanamido-4,7,10-trioxa-14,21-diaza-penta-triacontyl)-4,7,10,13,16-pentaoxanonadecane-1,19-diamide

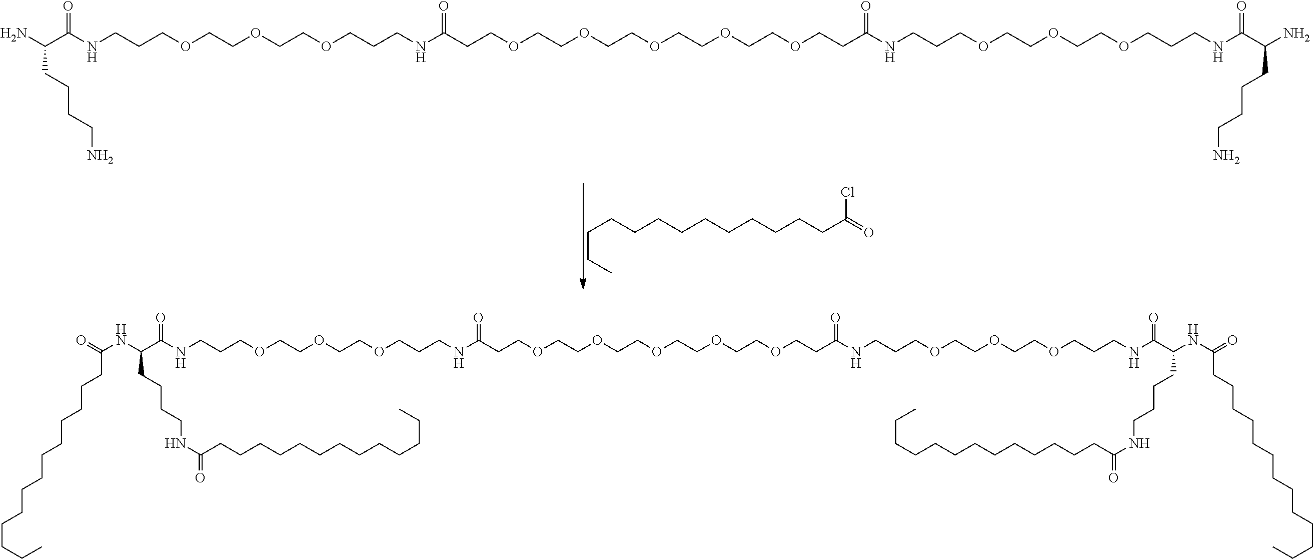 [SMILES: NCCCC[C@H](N)C(=O)NCCCOCCOCCOCCCNC(=O)CCOCCOCCOCCOCCOCCC(=O)NCCCOCCOCCOCCCNC(=O)[C@@H](N)CCCCN ; CCCCCCCCCCCCCC(=O)Cl ; CCCCCCCCCCCCCC(=O)NCCCC[C@@H](NC(=O)CCCCCCCCCCCCC)C(=O)NCCCOCCOCCOCCCNC(=O)CCOCCOCCOCCOCCOCCC(=O)NCCCOCCOCCOCCCNC(=O)[C@@H](CCCCNC(=O)CCCCCCCCCCCCC)NC(=O)CCCCCCCCCCCCC]

N1,N19-bis((S)-16,20-diamino-15-oxo-4,7,10-trioxa-14-azaicosyl)-4,7,10,13,16-pentaoxanonadecane-1,19-diamide (synthesis previously described) was dissolved in dichloromethane and placed in an ice-bath. Myristoyl chloride was added followed by triethylamine. The ice-bath was removed and the reaction was allowed to stir overnight at room temperature under a blanket of inert gas. Next day, diluted with dichloromethane to 100 mL and washed with 1M HCl (75 mL), H₂O (75 mL), saturated bicarbonate solution (75 mL) and saturated brine solution (75 mL). Back extracted all aqueous washes with dichloromethane (25 mL). Dried organics with MgSO₄, filtered and concentrated. Purification by silica gel chromatography with a dichloromethane/methanol gradient yielded N1,N19-bis((R)-15,22-dioxo-16-tetradecanamido-4,7,10-trioxa-14,21-diaza-penta-triacontyl)-4,7,10,13,16-pentaoxanonadecane-1,19-diamide (410 mg). LCMS ESI+: m/z 1841 (M+H).

### Example 32

### Synthesis of DiVA-242

### Preparation of N1,N16-bis((R,18E,20E,22E,24E)-11-((2E,4E,6E,8E)-3,7-dimethyl-9-(2,6,6-trimethyl-cyclohex-1-en-1-yl)nona-2,4,6,8-tetraenamido)-19,23-dimethyl-10,17-dioxo-25-(2,6,6-trimethylcyclohex-1-en-1-yl)-3,6-dioxa-9,16-diazapentacosa-18,20,22,24-tetraen-1-yl)-4,7,10,13-tetraoxahexadecane-1,16-diamide, also known as DIVA-242

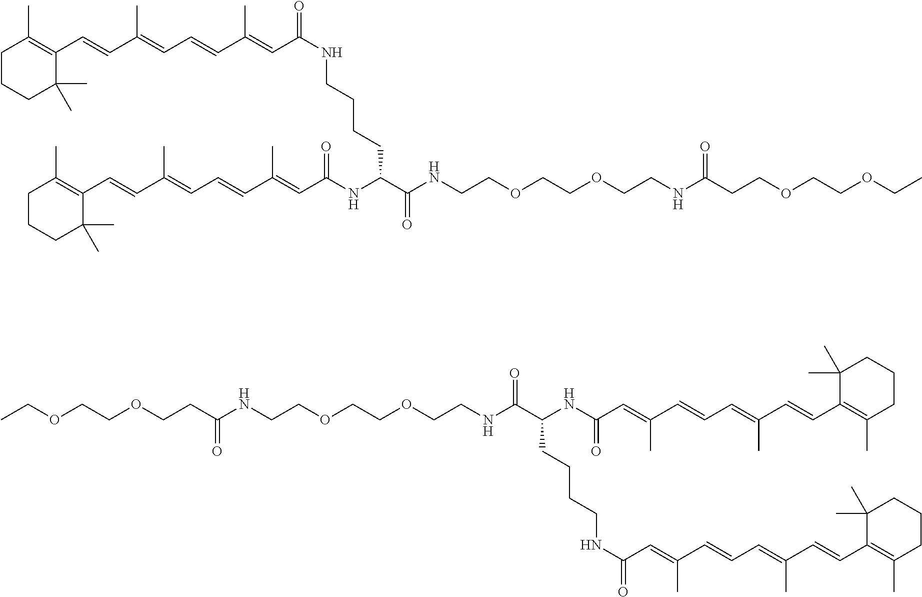

### Preparation of Intermediate 1: di-tert-butyl (10,25-dioxo-3,6,13,16,19,22,29,32-octaoxa-9,26-diazatetratriacontane-1,34-diyl)dicarbamate

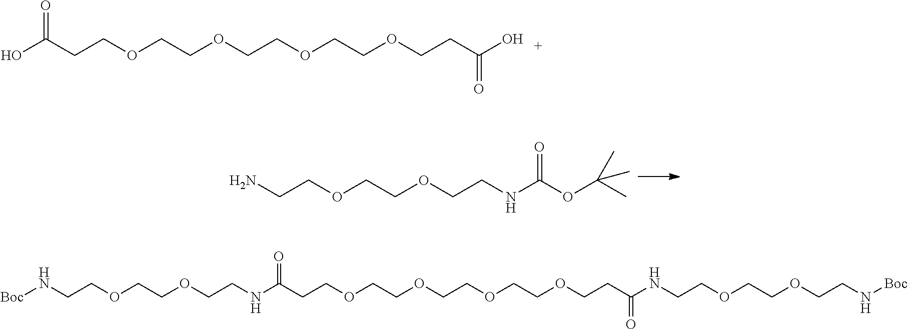 [SMILES: O=C(O)CCOCCOCCOCCOCCC(=O)O ; CC(C)(C)OC(=O)NCCOCCOCCN ; CC(C)(C)OC(=O)NCCOCCOCCNC(=O)CCOCCOCCOCCOCCC(=O)NCCOCCOCCNC(=O)OC(C)(C)C]

A round bottom flask containing dichloromethane (25 mL) was purged with inert gas and Bis-dPeg₄ acid (1000 mg, 3.40 mmol), N-Boc-3,6-dioxa-1,8-octane diamine (1816 μL, 7.65 mmol) and HOBt hydrate (1034 mg, 7.65 mmol) were added. NMM (841 μL, 7.65 mmol) was added to the suspension and the solution became clear. A suspension of EDC HCl salt (2249 mg, 11.7 mmol) & NMM (1121 μL, 10.2 mmol) in dichloromethane (25 mL) was added followed by DMAP (62 mg, 0.51 mmol). The reaction was allowed to stir overnight at room temperature. It was then diluted with dichloromethane to 100 mL and washed with H₂O (100 mL), 10% K₂CO₃ (100 mL) and saturated brine solution (100 mL), back extracted all aqueous washes with dichloromethane (30 mL), dried with MgSO₄, filtered and concentrated. Purification by silica gel chromatography with a dichloromethane/methanol gradient yielded di-tert-butyl (10,25-dioxo-3,6,13,16,19,22,29,32-octaoxa-9,26-diazatetratriacontane-1,34-diyedicarbamate (2.57 g).

### Preparation of intermediate 2: N1,N16-bis(2-(2-(2-aminoethoxy)ethoxy)ethyl)-4,7,10,13-tetraoxahexa-decane-1,16-diamide TFA salt

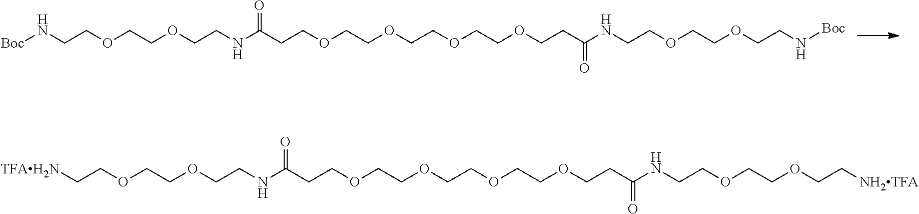

Di-tert-butyl (10,25-dioxo-3,6,13,16,19,22,29,32-octaoxa-9,26-diazatetratriacontane-1,34-diyl) dicarbamate was dissolved in dichloromethane (15 mL) and placed into an ice bath, The round bottom flask was flushed with inert gas and TFA (15 mL) was added. Mixture was allowed to stir for 20 minutes. Afterwards, the reaction mixture was concentrated to yield N1,N16-bis(2-(2-(2-aminoethoxy)ethoxy)ethyl)-4,7,10,13-tetraoxahexadecane-1,16-diamide TFA salt (1885 mg).

### Preparation of DIVA-242: N1,N16-bis((R,18E,20E,22E,24E)-11-((2E,4E,6E,8E)-3,7-dimethyl-9-(2,6,6-trimethylcyclohex-1-en-1-yl)nona-2,4,6,8-tetraenamido)-19,23-dimethyl-10,17-dioxo-25-(2,6,6-trimethylcyclohex-1-en-1-yl)-3,6-dioxa-9,16-diazapentacosa-18,20,22,24-tetraen-1-yl)-4,7,10,13-tetraoxahexadecane-1,16-diamide

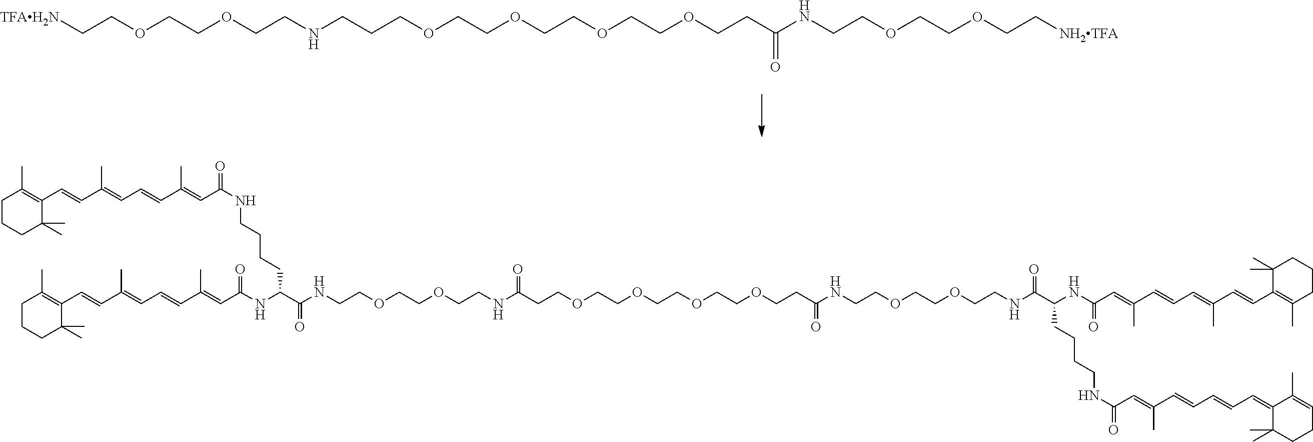

Synthesis of N1,N16-bis((R,18E,20E,22E,24E)-1142E,4E,6E,8E)-3,7-dimethyl-9-(2,6,6-trimethylcyclohex-1-en-1-yl)nona-2,4,6,8-tetraenamido)-19,23-dimethyl-10,17-dioxo-25-(2,6,6-trimethylcyclohex-1-en-1-yl)-3,6-dioxa-9,16-diazapentacosa-18,20,22,24-tetraen-1-yl)-4,7,10,13-tetraoxahexadecane-1,16-diamide (DIVA-242) follows the same protocol as diVA from N1,N16-bis(2-(2-(2-aminoethoxy)ethoxy)ethyl)-4,7,10,13-tetraoxahexadecane-1,16-diamide TFA salt. LCMS ESI+: m/z 1940 (M+H).

### Example 33

### Preparation of Fluorescent siRNA-Containing diVA-Bound Liposome

diVA-Liposome formulations were prepared using the method described above. The fluorescent siRNA was prepared by mixing 60% 2_M and 40% Dy647 labeled siRNA (obtained from Dharmacon). Details of 2M siRNA were as follows:

PS: 5′-idAB-rG-rA-rG-rA-rC-rA-rC-rA-rU-rG-rG-rG-rU-rG-25rC-25rU-25rA-25rU-25rA-C3-P-3′ (SEQ. ID NO: 13)

GS: 5′-mU-rA-mU-rA-mG-rC-25rA-rC-mC-rC-mA-rU-mG-rU-mG-rU-mC-rU-mC-C3-C3-3′ (SEQ. ID NO: 14)

wherein: rX represents ribonucleotides; mX represents 2′-O-methyl ribonucleotides; 25rX represents ribonucleotides with 2′-5′ linkages; C3 represents a 1,3-propanediol spacer; idAB represents inverted 1,2-dideoxy-D-ribose; P represents a phosphate group on the 3′-terminus. The 3′-terminus C3 is introduced by support-loaded 1,3-propanediol spacer. The 3′-terminus phosphate group (P) is introduced by the use of support-loaded diethyl sulfonyl (Pi) spacer. Dy647 labelled siRNA was a non-targeting control with RNA-induced silencing complex (RISC) free modification with Dy647 conjugated to the 5′ end. The above fluorescent siRNA was encapsulated in the diVA-liposome using the method described above, and fluorescent siRNA-containing diVA-bound liposome formations were prepared (hereinafter, referred to as ‘diVA-lip-siRNA-DY647’). The siRNA/lipid ratio (wt/wt) is 0.07, and the encapsulation efficiency of siRNA was around 97.5-99.1%.

In the same manner as above, fluorescent siRNA-containing liposome without diVA (lip-siRNA-DY647) was prepared.

### Example 34

### Uptake of diVA-lip-siRNA-DY647

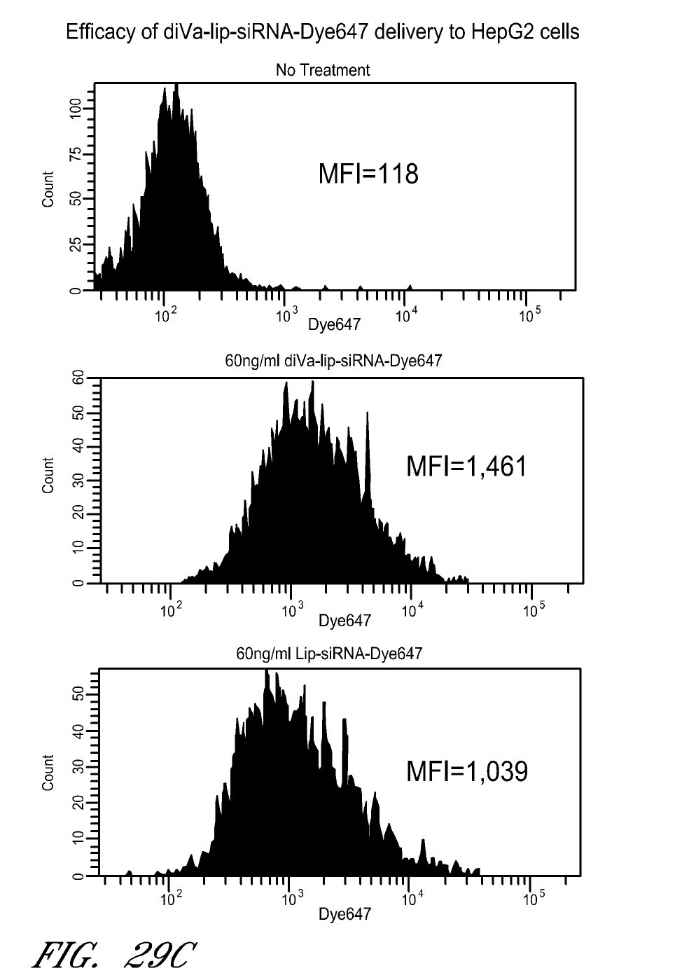

4.0×10⁴ cells of cancer cells A549, PANC-1, and HepG2 were plated on 6 cm dishes. 60 ng/ml of diVA-lip-siRNA-DY647 or lip-siRNA-DY647 was added into the plate, followed by the incubation in DMEM+10% FCS for 10 minutes at 37° C., 5% CO₂. Then the media were aspirated out and the cells were harvested. The cells were fixed by 4% paraformaldehyde (PFA) at room temperature for 15 minutes, and then washed by PBS once. After the washing, the cells were treated with 0.1% TritonX in PBS at room temperature for 5 minutes, and then washed by PBS three times. Then, cells were suspended in PBS with 1% bovine serum albumin (BSA). Fluorescence activated cell sorter (FACS) analysis was carried out using BD FACSCantoII to examine the uptake of cells in each sample (FIG. 29).

As is shown in FIGS. 29A, 29B and 29C, the mean fluorescence intensity (MFI) of diVA-lip-siRNA-DY647 was higher than lip-siRNA-DY647 in all of the tested cancer cell lines. These results clearly indicate that the specificity and transfer efficacy of the liposome is enhanced by diVA.

The above results show that the composition of the present invention is extremely effective in treatment of a cancer.

What is claimed is:

1. A targeting agent to a cancer cell, the targeting agent comprising one or more compounds selected from the group consisting of a retinoid, (retinoid)ₘ-linker-(retinoid)ₙ and (lipid)ₘ,linker-(retinoid)ₙ, wherein m and n are independently 0, 1, 2, or 3, except that m and n are not both zero; and wherein the linker comprises a polyethylene glycol (PEG) or PEG-like molecule.

2. The targeting agent according to claim 1, wherein at least one of the retinoid is selected from the group consisting of Vitamin A, retinoic acid, tretinoin, adapalene, 4-hydroxy(phenyl)retinamide (4-HPR), retinyl palmitate, retinal, saturated retinoic acid, and saturated, demethylated retinoic acid.

3. The targeting agent according to claim 1, wherein the retinoid is retinol.

4. The targeting agent according to claim 1, wherein the linker of the (retinoid)ₘ-linker-(retinoid)ₙ is selected from the group consisting of bis-amido-PEG, tris-amido-PEG, tetra-amido-PEG, Lys-bis-amido-PEG-Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

5. The targeting agent according to claim 1, wherein the (retinoid)ₘ-linker-(retinoid)ₙ is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

6. The targeting agent according to claim 5, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

7. The targeting agent according to claim 6, wherein the targeting agent comprises a compound of formula 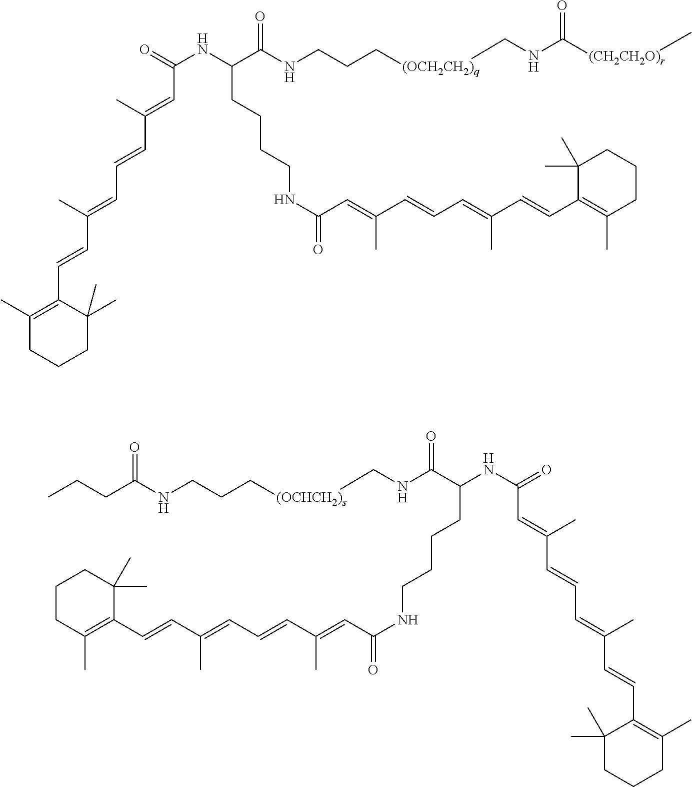
wherein q, r, and s are each independently 1, 2, 3, 4, 5, 6, 7, 8, 9, or 10.

8. The targeting agent according to claim 6, wherein the targeting agent comprises a compound in which q, r and s are 3, 5 and 3, respectively, of formula

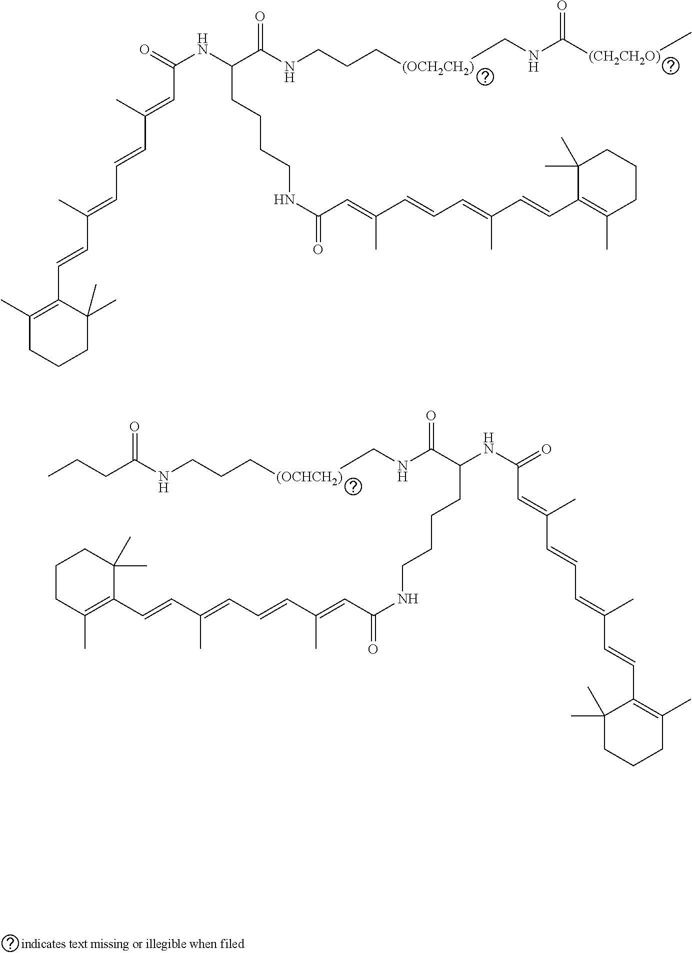

9. The targeting agent according to claim 1, wherein the lipid is selected from one or more of the group consisting of DODC, HEDODC, DSPE, DOPE, and DC-6-14.

10. The targeting agent according to claim 9, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

11. The targeting agent according to claim 9, wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

12. The targeting agent according to claim 11, selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

13. The targeting agent according to claim 1, wherein the lipid moieties comprise one or more lipids selected from the group consisting of HEDC, DODC, HEDC, HEDODC, DSPE, DOPE, and DC-6-14.

14. The targeting agent according to claim 13, wherein the lipid moieties further comprise S104.

15. A substance delivery carrier to a cancer cell, the carrier comprising the targeting agent according to claim 1.

16. The carrier according to claim 15, wherein the content of the targeting agent is 0.2 to 20 wt % of the entire carrier.

17. The carrier according to claim 15, wherein the molar ratio of the targeting agent to constituent components of the carrier other than the targeting agent is 8:1 to 1:4.

18. An anticancer composition comprising the targeting agent according to claim 1, and a drug that controls the activity or growth of a cancer cell.

19. The composition according to claim 18, wherein the drug that controls the activity or growth of a cancer cell is an anticancer agent.

20. An anticancer composition comprising the carrier according to claim 15, and a drug that controls the activity or growth of a cancer cell.

21. The composition according to claim 20, wherein the drug that controls the activity or growth of a cancer cell is an anticancer agent.

22. The composition according to claim 18, wherein the drug and the targeting agent are mixed at a place of medical treatment or in the vicinity thereof.

23. The composition according to claim 20, wherein the drug and the carrier are mixed at a place of medical treatment or in the vicinity thereof.

24. A preparation kit for the composition according to claim 18, wherein it comprises one or more containers comprising singly or in combination the drug, the targeting agent, and as necessary carrier constituent substances other than the targeting agent.

25. A targeting agent to a cancer-associated fibroblast, the targeting agent comprising one or more compounds selected from the group consisting of a retinoid, (retinoid)ₘ-linker-(retinoid)ₘ and (lipid)ₘ-linker-(retinoid)ₙ, wherein m and n are independently 0, 1, 2, or 3, except that m and n are not both zero; and wherein the linker comprises a polyethylene glycol (PEG) or PEG-like molecule.

26. The targeting agent according to claim 25, wherein at least one of the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

27. The targeting agent according to claim 25, wherein the retinoid is retinol.

28. The targeting agent according to claim 25, wherein the linker of the (retinoid)ₘ-linker-(retinoid)ₙ is selected from the group consisting of bis-amido-PEG, tris-amido-PEG, tetra-amido-PEG, Lys-bis-amido-PEG-Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

29. The targeting agent according to claim 25, wherein the (retinoid)ₘ-linker-(retinoid)ₙ is selected from the group consisting of retinoid-PEG-retinoid, (retinoid)₂-PEG-(retinoid)₂, VA-PEG2000-VA, (retinoid)₂-bis-amido-PEG-(retinoid)₂, and (retinoid)₂-Lys-bis-amido-PEG-Lys-(retinoid)₂.

30. The targeting agent according to claim 29, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

31. The targeting agent according to claim 30, wherein the targeting agent comprises a compound of formula 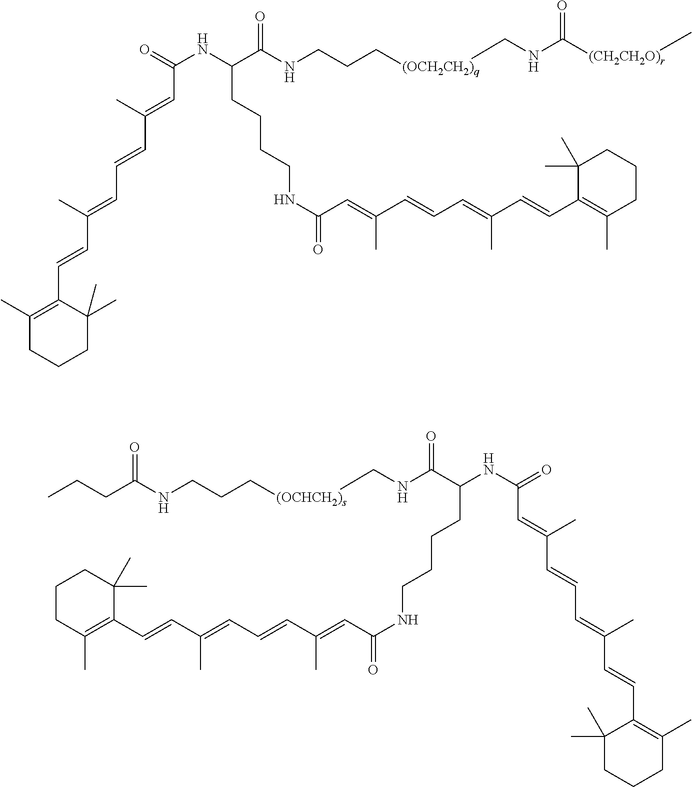
wherein q, r, and s are each independently 1, 2, 3, 4, 5, 6, 7, 8, 9, or 10.

32. The targeting agent according to claim 30, wherein the targeting agent comprises a compound of formula

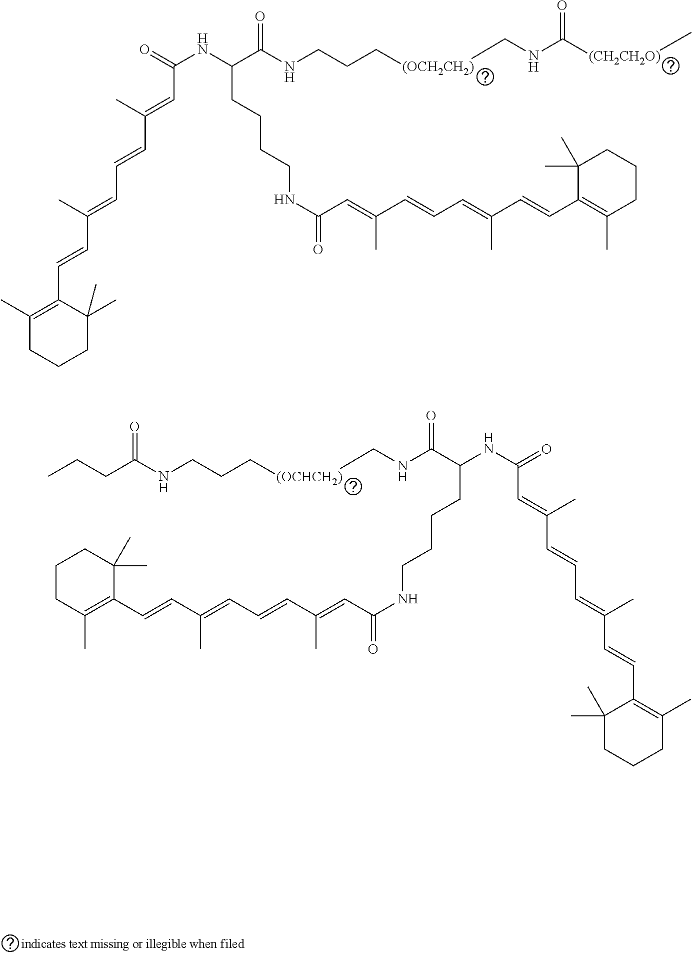

33. The targeting agent according to claim 25, wherein the lipid is selected from one or more of the group consisting of DODC, HEDODC, DSPE, DOPE, and DC-6-14.

34. The targeting agent according to claim 33, wherein the retinoid is selected from the group consisting of vitamin A, retinoic acid, tretinoin, adapalene, 4-hydroxy(phenyl)retinamide (4-HPR), retinyl palmitate, retinal, saturated retinoic acid, and saturated, demethylated retinoic acid.

35. The targeting agent according to claim 33, wherein the linker is selected from the group consisting of bis-amido-PEG, tris-amido-PEG, tetra-amido-PEG, Lys-bis-amido-PEG Lys, Lys-tris-amido-PEG-Lys, Lys-tetra-amido-PEG-Lys, Lys-PEG-Lys, PEG2000, PEG1250, PEG1000, PEG750, PEG550, PEG-Glu, Glu, C6, Gly3, and GluNH.

36. The targeting agent according to claim 35, selected from the group consisting of DSPE-PEG-VA, DSPE-PEG2000-Glu-VA, DSPE-PEG550-VA, DOPE-VA, DOPE-Glu-VA, DOPE-Glu-NH-VA, DOPE-Gly3-VA, DC-VA, DC-6-VA, and AR-6-VA.

37. The targeting agent according to claim 25, wherein the lipid moieties comprise one or more lipids selected from the group consisting of HEDC, DODC, HEDC, HEDODC, DSPE, DOPE, and DC-6-14.

38. The targeting agent according to claim 37, wherein the lipid moieties further comprise S104.

39. A substance delivery carrier to a cancer-associated fibroblast, the carrier comprising the targeting agent according to claim 25.

40. The carrier according to claim 39, wherein the content of the targeting agent is 0.2 to 20 wt % of the entire carrier.

41. The carrier according to claim 39, wherein the molar ratio of the targeting agent to constituent components of the carrier other than the targeting agent is 8:1 to 1:4.

42. An anti-cancer-associated fibroblast composition comprising the targeting agent according to claim 25, and a drug that controls the activity or growth of a cancer-associated fibroblast.

43. The composition according to claim 42, wherein the drug that controls the activity or growth of a cancer-associated fibroblast is selected from the group consisting of an inhibitor of activity or production of a bioactive substance selected from the group consisting of TGF-α, HGF, PDGF, VEGF, IGF, MMP, FGF, uPA, cathepsin, and SDF-1, a cell activity suppressor, a growth inhibitor, an apoptosis inducer, and an siRNA, ribozyme, antisense nucleic acid, DNA/RNA chimeric polynucleotide, or vector expressing same that targets one or more molecules from among an extracellular matrix constituent molecule produced by cancer-associated fibroblasts and a molecule involved in the production or secretion of the extracellular matrix constituent molecule.

44. The composition according to claim 43, wherein the molecule involved in the production or secretion of the extracellular matrix constituent molecule is HSP47.

45. An anti-cancer-associated fibroblast composition comprising the carrier according to claim 39, and a drug that controls the activity or growth of a cancer-associated fibroblast.

46. The composition according to claim 45, wherein the drug that controls the activity or growth of a cancer-associated fibroblast is selected from the group consisting of an inhibitor of activity or production of a bioactive substance selected from the group consisting of TGF-α, HGF, PDGF, VEGF, IGF, MMP, FGF, uPA, cathepsin, and SDF-1, a cell activity suppressor, a growth inhibitor, an apoptosis inducer, and an siRNA, ribozyme, antisense nucleic acid, DNA/RNA chimeric polynucleotide, or vector expressing same that targets one or more molecules from among an extracellular matrix constituent molecule produced by cancer-associated fibroblasts and a molecule involved in the production or secretion of the extracellular matrix constituent molecule.

47. The composition according to claim 46, wherein the molecule involved in the production or secretion of the extracellular matrix constituent molecule is HSP47.

48. The composition according to claim 42, wherein the drug and the targeting agent are mixed at a place of medical treatment or in the vicinity thereof.

49. The composition according to claim 45, wherein the drug and the carrier are mixed at a place of medical treatment or in the vicinity thereof.

50. A preparation kit for the composition according to claim 42, wherein it comprises one or more containers comprising singly or in combination the drug, the targeting agent, and as necessary carrier constituent substances other than the targeting agent.
